(19) United States
(12) Patent Application Publication
(10) Pub. No.: US 2025/0388691 A1
(43) Pub. Date: Dec. 25, 2025

(54) METHODS FOR TREATING LYMPHOMA

(71) Applicants: Xencor, Inc., Monrovia, CA (US); Incyte Corporation, Wilmington, DE (US)

(72) Inventors: William AINSWORTH, Evanston, IL (US); Steve KYE, Vernon Hills, IL (US); Michael CHIARELLA, Kendall Park, NJ (US); Raphael CLYNES, West Nyack, NY (US); David LIEBOWITZ, San Diego, CA (US)

(21) Appl. No.: 18/862,319

(22) PCT Filed: Apr. 10, 2023
(86) PCT No.: PCT/US2023/065591

§ 371 Date: Nov. 1, 2024

Related U.S. Application Data
(60) Provisional application No. 63/337,941, filed on May 3, 2022.
(60) Provisional application No. 63/340,909, filed on May 11, 2022.

(51) Int. Cl. C07K 16/28; A61K 31/454; A61K 39/00

(52) U.S. Cl. CPC C07K 16/2887; A61K 31/454; C07K 16/2803; C07K 16/2809; A61K 2039/545; C07K 2317/31; C07K 2317/565

(57) ABSTRACT

Provided herein, in certain aspects, are methods for the treatment of lymphoma, comprising administration of a CD19 antibody, a CD3×CD20 multispecific antibody, and 3-(4-amino-1-oxo 1,3-dihydro-2H-isoindol-2-yl) piperidine-2, 6-dione (Compound A).

### CROSS-REFERENCE TO RELATED APPLICATIONS

This application claims the benefit of U.S. Provisional Patent Application No. 63/337,941, filed May 3, 2022, and U.S. Provisional Patent Application No. 63/340,909, filed May 11, 2022, the content of each of which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application entitled “14718-043-228_SEQLISTING.xml”, was created on Apr. 5, 2023 and is 54,710 bytes in size.

### 1. FIELD

Provided herein, in certain aspects, are methods for treating a lymphoma using a combination of a CD19 antibody, a CD3×CD20 multispecific antibody, and a compound having the structure

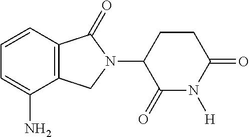 [SMILES: Nc1cccc2c1CN(C1CCC(=O)NC1=O)C2=O]

(i.e., 3-(4-amino-1-oxo 1,3-dihydro-2H-isoindol-2-yl) piperidine-2,6-dione (Compound A)), or, for example, a pharmaceutically acceptable salt, solvate, or stereoisomer thereof of Compound A.

### 2. SUMMARY

In one aspect, provided herein is a method of treating lymphoma in a subject in need thereof, comprising administering to the subject: (a) an antibody that binds CD19 (CD19 antibody); (b) a multispecific antibody, wherein the multispecific antibody comprises a first binding domain that binds to CD3 and a second binding domain that binds to CD20 (CD3×CD20 antibody); and (c) a compound having the structure:

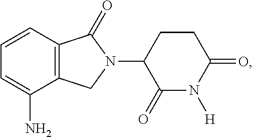 [SMILES: Nc1cccc2c1CN(C1CCC(=O)NC1=O)C2=O]

(i.e., 3-(4-amino-1-oxo 1,3-dihydro-2H-isoindol-2-yl) piperidine-2,6-dione (Compound A)), or a pharmaceutically acceptable salt, solvate, or stereoisomer thereof. In specific embodiments, the subject is administered a first dose of the CD19 antibody at least one day prior to administration to the subject of: (i) a first dose of Compound A or pharmaceutically acceptable salt, solvate or stereoisomer thereof; or (ii) a first dose of the CD3×CD20 antibody.

In one embodiment, the CD19 antibody comprises: (i) a heavy chain variable (VH) domain comprising a VH complementarity determining region (CDR) 1, a VH CDR2, and a VH CDR3 having an amino acid sequence of SEQ ID NO:1, SEQ ID NO:2, and SEQ ID NO:3, respectively; and (ii) a light chain variable (VL) domain comprising a VL CDR1, a VL CDR2, and VL CDR3 having an amino acid sequence of SEQ ID NO:4, SEQ ID NO:5, and SEQ ID NO:6, respectively. In one embodiment, the first binding domain of the CD3×CD20 antibody that binds to CD3 comprises: (i) a VH domain comprising a VH CDR1, a VH CDR2, and a VH CDR3 having an amino acid sequence of SEQ ID NO:7, SEQ ID NO:8, and SEQ ID NO:9, respectively; and (ii) a VL domain comprising a VL CDR1, a VL CDR2, and VL CDR3 having an amino acid sequence of SEQ ID NO:10, SEQ ID NO:11, and SEQ ID NO:12, respectively. In one embodiment, the second binding domain of the CD3×CD20 antibody that binds to CD20 comprises: (i) a VH domain comprising a VH CDR1, VH CDR2, and a VH CDR3 having an amino acid sequence of SEQ ID NO:13, SEQ ID NO:14, and SEQ ID NO:15, respectively; and (ii) a VL domain comprising a VL CDR1, VL CDR2, and VL CDR3 having an amino acid sequence of SEQ ID NO:16, SEQ ID NO:17, and SEQ ID NO:18, respectively. In another one embodiment, (a) the CD19 antibody comprises: (i) a VH CDR1, a VH CDR2, and a VH CDR3 having an amino acid sequence of SEQ ID NO:1, SEQ ID NO:2, and SEQ ID NO:3, respectively; and (ii) a VL CDR1, a VL CDR2, and VL CDR3 having an amino acid sequence of SEQ ID NO:4, SEQ ID NO:5, and SEQ ID NO:6, respectively; the (b) the first binding domain of the CD3×CD20 antibody that binds to CD3 comprises: (i) a VH domain comprising a VH CDR1, a VH CDR2, and a VH CDR3 having an amino acid sequence of SEQ ID NO:7, SEQ ID NO:8, and SEQ ID NO:9, respectively; and (ii) a VL domain comprising a VL CDR1, a VL CDR2, and VL CDR3 having an amino acid sequence of SEQ ID NO:10, SEQ ID NO:11, and SEQ ID NO:12, respectively; and (c) the second binding domain of the CD3×CD20 antibody that binds to CD20 comprises: (i) a VH domain comprising a VH CDR1, VH CDR2, and a VH CDR3 having an amino acid sequence of SEQ ID NO:13, SEQ ID NO:14, and SEQ ID NO:15, respectively; and (ii) a VL domain comprising a VL CDR1, VL CDR2, and VL CDR3 having an amino acid sequence of SEQ ID NO:16, SEQ ID NO:17, and SEQ ID NO:18, respectively.

In one embodiment, the CD19 antibody comprises a VH domain having an amino acid sequence that is about 90%, 95%, 98%, 99%, or 100% identical to the amino acid sequence of SEQ ID NO:19. In another embodiment, the CD19 antibody comprises a VL domain having an amino acid sequence that is about 90%, 95%, 98%, 99%, or 100% identical to the amino acid sequence of SEQ ID NO:20. In one embodiment, the first binding domain of the CD3×CD20 antibody that binds to CD3 comprises a VH domain having an amino acid sequence that is about 90%, 95%, 98%, 99%, or 100% identical to the amino acid sequence of SEQ ID NO:21.

In one embodiment, the first binding domain of the CD3×CD20 antibody that binds to CD3 comprises a VL domain having an amino acid sequence that is about 90%, 95%, 98%, 99%, or 100% identical to the amino acid sequence of SEQ ID NO:22. In one embodiment, the second binding domain of the CD3×CD20 antibody that binds to CD20 comprises a VH domain having an amino acid sequence that is about 90%, 95%, 98%, 99%, or 100% identical to the amino acid sequence of SEQ ID NO:23. In one embodiment, the second binding domain of the CD3×CD20 antibody that binds to CD20 comprises a VL domain having an amino acid sequence that is about 90%, 95%, 98%, 99%, or 100% identical to the amino acid sequence of SEQ ID NO:24.

In one embodiment, the CD3×CD20 antibody comprises: (a) a first monomer comprising, from N- to C-terminus, a scFv-linker-CH2-CH3 having the amino acid sequence of SEQ ID NO:25; (b) a second monomer comprising, from N- to C-terminus, a VH-CH1-hinge-CH2-CH3 having the amino acid sequence of SEQ ID NO:26; and (c) a third monomer comprising, from N- to C-terminus, a VL-CL having the amino acid sequence of SEQ ID NO:27.

In one embodiment, the compound is

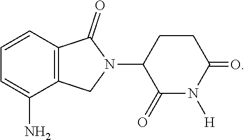 [SMILES: Nc1cccc2c1CN(C1CCC(=O)NC1=O)C2=O]

(i.e., 3-(4-amino-1-oxo 1,3-dihydro-2H-isoindol-2-yl) piperidine-2,6-dione (Compound A)).

In one embodiment, the lymphoma is Non-Hodgkin lymphoma. In one embodiment, the Non-Hodgkin lymphoma is Diffuse Large Cell Lymphoma (DLBCL). In one embodiment, the DLBCL is relapsed, refractory, or relapsed and refractory DLBCL. In one embodiment, the DLBCL is primary refractory DLBCL. In one embodiment, wherein the DLBCL is first line DLBCL. In one embodiment, the lymphoma is a CD20-expressing lymphoma. In one embodiment, the lymphoma is a CD19-expressing lymphoma. In one embodiment, the subject has not received stem cell transplantation. In one embodiment, the subject is not eligible for stem cell transplantation. In one embodiment, the stem cell transplantation is autologous stem cell transplantation.

In one embodiment, the method comprises cyclic administration of the CD19 antibody, Compound A or pharmaceutically acceptable salt, solvate, or stereoisomer thereof, and the CD3×CD20 antibody. In one embodiment, each cycle of the cyclic administration is 28 days. In one embodiment, the cyclic administration comprises about one cycle, two cycles, three cycles, four cycles, five cycles, six cycles, seven cycles, eight cycles, nine cycles, ten cycles, eleven cycles, twelve cycles, or more than twelve cycles.

In one embodiment, the first dose of the CD19 antibody is administered to the subject prior to day 1 of the first cycle of the cyclic administration. In one embodiment, the first dose of the CD19 antibody is administered to the subject at least one day, two days, three days, four days, five days, six days, seven days, eight days, nine days, ten days, or more than ten days prior to day 1 of the first cycle of the cyclic administration. In one embodiment, the first dose of the CD19 antibody is administered to the subject four days prior to day 1 of the first cycle of the cyclic administration. In one embodiment, the CD19 antibody is administered to the subject eight days prior to day 1 of the first cycle of the cyclic administration. In one embodiment, the CD19 antibody is administered to the subject four days and eight days prior to day 1 of the first cycle of the cyclic administration. In one embodiment, the CD19 antibody is administered to the subject on day(s) 1, 8, 15, and/or 22 of a cycle of the cyclic administration. In one embodiment, the CD19 antibody is administered to the subject on days 1, 8, 15, and 22 of a cycle of the cyclic administration. In one embodiment, CD19 antibody is administered to the subject on days 1 and 15 of a cycle of the cyclic administration. In one embodiment, the CD19 antibody is administered to the subject on days 1, 8, 15, and 22 of each of cycles 1-3 of the cyclic administration. In one embodiment, the CD19 antibody is administered to the subject on days 1 and 15 for cycle 4 and onwards of the cyclic administration. In one embodiment, the CD19 antibody is administered to the subject on days 1 and 15 for cycles 4-6 of the cyclic administration. In one embodiment, the CD19 antibody is administered to the subject every 6 to 8 days in a cycle of the cyclic administration.

In one embodiment, the Compound A or pharmaceutically acceptable salt, solvate, or stereoisomer thereof is administered to the subject for 21 continuous days of the cyclic administration. In one embodiment, the Compound A or pharmaceutically acceptable salt, solvate, or stereoisomer thereof is administered to the subject from day 1 to day 21 of the cyclic administration. In one embodiment, the Compound A or pharmaceutically acceptable salt, solvate, or stereoisomer thereof is administered to the subject for 21 days followed by seven days of rest in a 28 day cycle of the cyclic administration.

In one embodiment, the CD3×CD20 antibody is administered to the subject on day(s) 1, 8, 15, and/or 22 of a cycle of the cyclic administration. In one embodiment, the CD3×CD20 antibody is administered to the subject on days 1, 8, 15, and 22 of a cycle of the cyclic administration. In one embodiment, the CD3×CD20 antibody is administered to the subject on days 1 and 15 of a cycle of the cyclic administration. In one embodiment, the CD3×CD20 antibody is administered to the subject on days 1, 8, 15, and 22 of cycle 1 and cycle 2 of the cyclic administration. In one embodiment, the CD3×CD20 antibody is administered to the subject on days 1 and 15 for cycle 3 and onwards of the cyclic administration. In one embodiment, the CD3×CD20 antibody is administered to the subject on days 1 and 15 for cycles 3-6 of the cyclic administration. In one embodiment, CD3×CD20 antibody is administered to the subject every 6 to 8 days in a cycle of the cyclic administration.

In one embodiment, the CD19 antibody is administered to the subject in an amount of about 1 mg/kg to about 20 mg/kg per day. In one embodiment, the CD19 antibody is administered to the subject in an amount of about 12 mg/kg per day. In one embodiment, the CD19 antibody is administered to the subject in an amount of about 5 mg/kg per day. In one embodiment, the CD19 antibody is administered to the subject in an amount of about 10 mg/kg per day.

In one embodiment, the Compound A or pharmaceutically acceptable salt, solvate, or stereoisomer thereof is administered to the subject in an amount of about 1 mg to about 30 mg per day. In one embodiment, the Compound A or pharmaceutically acceptable salt, solvate, or stereoisomer thereof is administered to the subject in an amount of about 2.5 mg, 5 mg, 10 mg, 15 mg, 20 mg, or 25 mg per day. In one embodiment, the Compound A or pharmaceutically acceptable salt, solvate, or stereoisomer thereof is administered to the subject in an amount of about 2.5 mg per day. In one embodiment, the Compound A or pharmaceutically acceptable salt, solvate, or stereoisomer thereof is administered to the subject in an amount of about 5 mg per day. In one embodiment, the Compound A or pharmaceutically acceptable salt, solvate, or stereoisomer thereof is administered to the subject in an amount of about 10 mg per day. In one embodiment, the Compound A or pharmaceutically acceptable salt, solvate, or stereoisomer thereof is administered to the subject in an amount of about 15 mg per day. In one embodiment, the Compound A or pharmaceutically acceptable salt, solvate, or stereoisomer thereof is administered to the subject in an amount of about 20 mg per day. In one embodiment, the Compound A or pharmaceutically acceptable salt, solvate, or stereoisomer thereof is administered to the subject in an amount of about 25 mg per day.

In one embodiment, the CD3×CD20 antibody is administered to the subject in an amount of about 0.8 mg to about 100 mg per day. In one embodiment, the CD3×CD20 antibody is administered to the subject in an amount of about 0.8 mg to about 50 mg per day. In one embodiment, the CD3×CD20 antibody is administered to the subject in an amount of about 0.8 mg to about 20 mg per day. In one embodiment, the CD3×CD20 antibody is administered to the subject in an amount of about 0.8 mg per day. In one embodiment, the CD3×CD20 antibody is administered to the subject in an amount of about 2 mg per day. In one embodiment, the CD3×CD20 antibody is administered to the subject in an amount of about 20 mg per day. In one embodiment, the CD3×CD20 antibody is administered to the subject in an amount of about 35 mg per day. In one embodiment, wherein the CD3×CD20 antibody is administered to the subject in an amount of about 50 mg per day.

In one embodiment, the first dose of the CD3×CD20 antibody is on day 1 of the first cycle of the cyclic administration. In one embodiment, the CD3×CD20 antibody is administered to the subject in an amount of about 0.8 mg on day 1 of the first cycle, about 2 ng on day 8 of the first cycle, about 20 mg on days 15 and 22 of the first cycle, and about 20 mg per day for any subsequent cycles. In one embodiment, the CD3×CD20 antibody is administered to the subject in an amount of about 0.8 mg on day 1 of the first cycle, about 2 mg on day 8 of the first cycle, about 20 mg on day 15 of the first cycle, about 35 mg on day 22 of the first cycle, and about 50 mg per day for any subsequent cycles.

In one embodiment, the first dose of Compound A or pharmaceutically acceptable salt, solvate, or stereoisomer thereof is on day 1 of the first cycle of the cyclic administration.

In one embodiment, the first dose of the CD3×CD20 antibody and the first dose of Compound A or pharmaceutically acceptable salt, solvate, or stereoisomer thereof are both on day 1 of the first cycle of the cyclic administration.

In one embodiment, the CD19 antibody is administered to the subject once a week in a cycle of the cyclic administration. In one embodiment, the CD19 antibody is administered to the subject once a week for cycles 1-3 of the cyclic administration. In one embodiment, the CD19 antibody is administered to the subject every two weeks in a cycle of the cyclic administration. In one embodiment, the CD19 antibody is administered to the subject every two weeks for cycles 4 and onwards of the cyclic administration. In one embodiment, wherein the CD3×CD20 antibody is administered to the subject once a week in a cycle of the cyclic administration. In one embodiment, wherein the CD3×CD20 antibody is administered to the subject once a week for cycles 1 and 2 of the cyclic administration. In one embodiment, wherein the CD3×CD20 antibody is administered to the subject every two weeks in a cycle of the cyclic administration. In one embodiment, wherein the CD3×CD20 antibody is administered to the subject every two weeks for cycles 3 and onwards of the cyclic administration. In one embodiment, wherein the CD3×CD20 antibody and the CD19 antibody are each administered to the subject in at most 4 days in a cycle of the cyclic administration.

In one embodiment, the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 20 mg, wherein about 20 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 35 mg, wherein about 50 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 20 mg, wherein about 20 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 35 mg, wherein about 50 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment, and wherein each cycle of the cyclic administration is 28 days.

In one embodiment, the method comprises cyclic administration of the CD19 antibody to the subject, wherein from about 10 mg/kg to about 15 mg/kg of the CD19 antibody is administered to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during a course of treatment, wherein from about 10 mg/kg to about 15 mg/kg of the CD19 antibody is administered to the subject 8 days and 4 days prior to day 1 of a first cycle of the cyclic administration, and wherein each cycle of the cyclic administration is 28 days.

In one embodiment, the method comprises cyclic administration of the CD19 antibody to the subject, wherein about 12 mg/kg of the CD19 antibody is administered to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during a course of treatment, wherein about 12 mg/kg of the CD19 antibody is administered to the subject 8 days and 4 days prior to day 1 of a first cycle of the cyclic administration, and wherein each cycle of the cyclic administration is 28 days.

In one embodiment, the method comprises cyclic administration of Compound A or pharmaceutically acceptable salt, solvate, or stereoisomer thereof to the subject, wherein about 25 mg of Compound A or pharmaceutically acceptable salt, solvate, or stereoisomer thereof is administered to the subject on day 1 and on every day for 21 days followed by 7 days of rest in each cycle of the cyclic administration, and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering from about 0.6 to about 1 mg of the CD3×CD20 antibody to the subject on day 1, from about 1.8 mg to about 2.2 mg on about day 8, and from about 18 mg to about 22 mg on about day 15 and 22 of the first cycle; administering about 20 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment; (b) administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration; and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering from about 0.6 to about 1 mg of the CD3×CD20 antibody to the subject on day 1, from about 1.8 mg to about 2.2 mg on about day 8, from about 18 mg to about 22 mg on about day 15, and from about 33 mg to about 36 mg on about day 22 of the first cycle; administering about 50 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment; (b) administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration; and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering about 0.8 mg of the CD3×CD20 antibody to the subject on day 1, about 2 mg on about day 8, and about 20 mg on day 15 and 22 of the first cycle; administering about 20 mg of the CD3×CD20 antibody to the subject on day 1 and every 7 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment; (b) administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering about 0.8 mg of the CD3×CD20 antibody to the subject on day 1, about 2 mg on about day 8, about 20 mg on day 15, and about 35 mg on day 22 of the first cycle; administering about 50 mg of the CD3×CD20 antibody to the subject on day 1 and every 7 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment; (b) administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering about 0.8 mg of the CD3×CD20 antibody to the subject on day 1, about 2 mg on day 8, and about 20 mg on days 15 and 22 of the first cycle; administering about 20 mg of the CD3×CD20 antibody to the subject on days 1, 8, 15, and 22 of the second cycle of the cyclic administration, wherein about 20 mg of the CD3×CD20 antibody is administered to the subject on days 1 and 15 of cycle 3 and of any subsequent cycle during a course of treatment; (b) administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

In one embodiment, the method comprises cyclic administration of the CD19 antibody, the CD3×CD20 antibody, and Compound A or pharmaceutically acceptable salt, solvate, or stereoisomer thereof to the subject, and wherein the method comprises: (a) administering about 0.8 mg of the CD3×CD20 antibody to the subject on day 1, about 2 mg on day 8, about 20 mg on day 15, and about 35 mg on day 22 of the first cycle; administering about 50 mg of the CD3×CD20 antibody to the subject on days 1, 8, 15, and 22 of the second cycle of the cyclic administration, wherein about 50 mg of the CD3×CD20 antibody is administered to the subject on days 1 and 15 of cycle 3 and of any subsequent cycle during a course of treatment; (b) administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and (c) administering about 25 mg of Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

In one embodiment, the CD19 antibody is tafasitamab. In one embodiment, the CD19 antibody is a biosimilar of tafasitamab. In one embodiment, the CD19 antibody is a bioequivalent of tafasitamab. In one embodiment, the CD3×CD20 antibody is plamotamab. In one embodiment, the CD3×CD20 antibody is a biosimilar of plamotamab. In one embodiment, the CD3×CD20 antibody is a bioequivalent of plamotamab. In one embodiment, the Compound A is lenalidomide. In some embodiments, the compound is a pharmaceutically acceptable salt of lenalidomide. In some embodiments, the compound is a pharmaceutically acceptable solvate of lenalidomide. In some embodiments, the compound is a pharmaceutically acceptable stereoisomer of lenalidomide.

In one embodiment, the Compound A or pharmaceutically acceptable salt, solvate, or stereoisomer thereof is administered orally to the subject. In one embodiment, the Compound A or pharmaceutically acceptable salt, solvate, or stereoisomer thereof is administered in a capsule or tablet to the subject.

In one embodiment, the CD19 antibody is administered intravenously.

In one embodiment, the CD19 antibody is not administered to the subject on day 4 of the first cycle of the cyclic administration.

In one embodiment, the CD3×CD20 antibody is not administered to the subject on day 4 of the first cycle of the cyclic administration.

In one embodiment, the method further comprises determining positron emission tomography-computed tomography (PET-CT) after every two cycles of the cyclic administration.

In one embodiment, the subject has received a prior CAR-T therapy.

In one embodiment, the method results in enhanced therapeutic efficacy relative to administration of both the CD19 antibody and Compound A or pharmaceutically acceptable salt, solvate, or stereoisomer thereof to the subject, but not the CD3×CD20 antibody. In one embodiment, the enhanced therapeutic efficacy is measured by increased overall survival time. In one embodiment, the enhanced therapeutic efficacy is measured by increased progression-free survival. In one embodiment, the enhanced therapeutic efficacy is measured by a decrease in the number of cancer cells in a biological sample obtained from the subject as compared to a reference. In one embodiment, the reference is the number of cancer cells in a biological sample obtained from the subject at an earlier time point. In one embodiment, the reference is a predetermined value. In one embodiment, the reference is the number of cancer cells in a biological sample obtained from another subject with lymphoma. In one embodiment, the reference is the number of cancer cells in a biological sample obtained from a population of subjects with lymphoma. In one embodiment, the biological sample is blood. In one embodiment, the biological sample is serum. In one embodiment, the biological sample is plasma. In one embodiment, the enhanced therapeutic efficacy is measured by an improved overall response rate and/or increased quality of life of the subject.

In one embodiment, the subject received a prior treatment for lymphoma. In one embodiment, the prior treatment comprises chemoimmunotherapy. In one embodiment, the prior treatment comprises administration of an anti-CD20 antibody. In one embodiment, the prior treatment comprises chemoimmunotherapy and administration of an anti-CD20 antibody.

In one aspect, provided herein is a method of treating lymphoma in a subject in need thereof, comprising administering to the subject: (a) an antibody that binds CD19 (CD19 antibody); (b) a multispecific antibody, wherein the multispecific antibody comprises a first binding domain that binds to CD3 and a second binding domain that binds to CD20 (CD3×CD20 antibody); and (c) a compound having the structure:

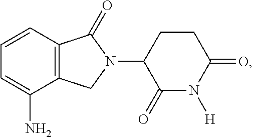 [SMILES: Nc1cccc2c1CN(C1CCC(=O)NC1=O)C2=O]

(i.e., 3-(4-amino-1-oxo 1,3-dihydro-2H-isoindol-2-yl) piperidine-2,6-dione (Compound A)), or a pharmaceutically acceptable salt, solvate, or stereoisomer thereof.

In one embodiment, (a) the CD19 antibody comprises: (i) a heavy chain variable (VH) domain comprising a VH complementarity determining region (CDR) 1, a VH CDR2, and a VH CDR3 having an amino acid sequence of SEQ ID NO:1, SEQ ID NO:2, and SEQ ID NO:3, respectively; and (ii) a light chain variable (VL) domain comprising a VL CDR1, a VL CDR2, and VL CDR3 having an amino acid sequence of SEQ ID NO:4, SEQ ID NO:5, and SEQ ID NO:6, respectively; (b) the multispecific antibody comprises: (i) an anti-CD3e heavy chain having an amino acid sequence of SEQ ID NO:30, an anti-CD3e light chain having an amino acid sequence of SEQ ID NO:31, an anti-CD20 heavy chain having an amino acid sequence of SEQ ID NO:32 and an anti-CD20 light chain having an amino acid sequence of SEQ ID NO:33, or (ii) an anti-CD3e heavy chain having an amino acid sequence of SEQ ID NO:34, an anti-CD3e light chain having an amino acid sequence of SEQ ID NO:35, an anti-CD20 heavy chain having an amino acid sequence of SEQ ID NO:36 and an anti-CD20 light chain having an amino acid sequence of SEQ ID NO:37, or (iii) an anti-CD3 heavy chain having an amino acid sequence of SEQ ID NO:38, a light chain having an amino acid sequence of SEQ ID NO:39, and an anti-CD20 heavy chain having an amino acid sequence of SEQ ID NO:40, or (iv) an anti-CD20/CD3 heavy chain having an amino acid sequence of SEQ ID NO:41, an anti-CD3e light chain having an amino acid sequence of SEQ ID NO:42, an anti-CD20 heavy chain having an amino acid sequence of SEQ ID NO:43 and an anti-CD20 light chain having an amino acid sequence of SEQ ID NO:44.

In one embodiment, (a) the CD19 antibody comprises a VH domain having an amino acid sequence that is about or at least about 90%, 95%, 98%, 99%, or 100% identical to the amino acid sequence of SEQ ID NO:19; (b) the CD19 antibody comprises a VL domain having an amino acid sequence that is about or at least about 90%, 95%, 98%, 99%, or 100% identical to the amino acid sequence of SEQ ID NO:20.

In one embodiment, the compound is

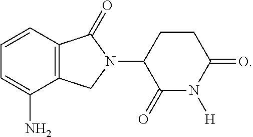 [SMILES: Nc1cccc2c1CN(C1CCC(=O)NC1=O)C2=O]

In one embodiment, the lymphoma is Non-Hodgkin lymphoma. In one embodiment, the Non-Hodgkin lymphoma is Diffuse Large Cell Lymphoma (DLBCL). In one embodiment, the DLBCL is relapsed, refractory, or relapsed and refractory DLBCL. In one embodiment, the DLBCL is primary refractory DLBCL. In one embodiment, the DLBCL is first line DLBCL.

In one embodiment, the subject achieves a complete metabolic response as determined by a positron emission tomography (PET)-computed tomography (CT) scan. In one embodiment, the subject achieves a complete metabolic response on or after: cycle 2, cycle 4, cycle 6, cycle 8, and/or end of treatment. In one embodiment, the subject achieves a complete metabolic response on or after: day 26 of cycle 2, day 26 of cycle 4, day 26 of cycle 6, day 26 of cycle 8, and/or end of treatment. In one embodiment, the subject achieves a complete metabolic response on or after: 61 days, 117 days, 177 days, 233 days, or 299 days from a first administration of the multispecific antibody to the subject.

### 3. BRIEF DESCRIPTION OF THE DRAWINGS

FIG. 1 depicts the structure of the anti-CD3×anti-CD20 multispecific antibody described herein. The multispecific antibody has a “bottle opener” format (also referred to as the “triple F” format). Bottle opener format antibodies include a) a first monomer that includes a first Fc domain and an scFv region, wherein the scFv includes a first variable heavy chain and a first variable light chain (also referred herein as a “scFv-Fc heavy chain;” b) a second monomer that includes a VH-CH1-hinge-CH2-CH3, wherein VH is a second variable heavy chain and CH2 and CH3 is a second Fc domain (also referred herein as a “Fab-Fc heavy chain;” and c) a light chain that includes a second variable light chain. As shown in FIG. 1, the scFv is the CD3 binding domain and the second variable heavy chain and second variable light chain comprise the CD20 binding domain.

FIG. 2 is a schematic of dosing regimen for combination therapy using a CD19 antibody, an anti-CD3×anti-CD20 multispecific antibody, and Compound A.

FIG. 3 depicts the Part 1 and Part 2 dose and schedule treatment regimes.

### 4. DETAILED DESCRIPTION

### 4.1 Definitions

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

Unless otherwise indicated, the term “at least” preceding a series of elements is to be understood to refer to every element in the series.

Those skilled in the art will recognize or be able to ascertain using no more than routine experimentation, many equivalents to the specific embodiments described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “comprise,” “comprises,” or “comprising,” as used herein, may be replaced with “consists of,” “consisting of,” “consists essentially of,” or “consisting essentially of.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “CD3,” also known as “cluster of differentiation 3,” refers to a T-cell co-receptor that helps in activation of both cytotoxic T-cell (e.g., CD8+ naïve T cells) and T helper cells (e.g., CD4+ naïve T cells) and is composed of four distinct chains: one CD37 chain (e.g., Genbank Accession Numbers NM_000073 and MP_000064 (human)), one CD36 chain (e.g., Genbank Accession Numbers NM_000732, NM_001040651, NP_00732 and NP_001035741 (human)), and two CD3E chains (e.g., Genbank Accession Numbers NM_000733 and NP_00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes. In a specific embodiment, the CD3 is human CD3.

As used herein, “CD20,” also known as “B-lymphocyte antigen CD20,” “CD20 antigen,” “CD20 Receptor,” “Membrane Spanning 4-Domains A1,” “Membrane-Spanning 4-Domains, Subfamily A, Member 1,” “Leukocyte Surface Antigen Leu-16,” “Bp35,” “B-Lymphocyte Cell-Surface Antigen 1,” “LEU-16,” “CVID5,” “MS4A,” “Bl,” and “S7,” refers to an activated-glycosylated phosphoprotein expressed on the surface of B-cells and is encoded by the MS4A1 gene in humans (e.g., Genbank Accession Numbers NM_152866, NM_021950, NP_068769 and NP_690605 (human)). CD20 plays a role in the development and differentiation of B-cells into plasma cells. In a specific embodiment, the CD20 is human CD20.

As used herein, “CD19,” also known as “B cell surface antigen B4,” “B-cell antigen CD19,” “CD19 antigen,” and “Leu-12,” refers to a cell surface protein expressed by B cells and encoded by the gene designated CD19 (e.g., HGNC: 1633; NCBI Entrez Gene: 930; Ensembl: ENSG00000177455; OMIM®: 107265; UniProtKB/Swiss-Prot: P15391). CD19 is involved with B cell activation and signaling pathways. In a specific embodiment, the CD19 is human CD19.

As used herein, “lenalidomide” refers to the thalidomide analogue 3-(4-amino-1-oxo-1,3-dihydro-isoindol-2-yl)-piperidine-2,6-dione (Compound A), or a pharmaceutically acceptable salt, solvate, or stereoisomer thereof. Lenalidomide is an immunomodulatory agent that has antiangiogenic and antineoplastic properties.

The term “bispecific antibody” or “multispecific antibody,” as used herein, means any non-native or alternate antibody format that engages two or more different antigens (e.g., CD3×CD20 multispecific antibodies).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. In specific embodiments, the antibodies provided herein are IgG. In other embodiments, the antibodies provided herein are IgG1. Antibody light chains of vertebrate species can be assigned to one of two clearly distinct types, namely kappa and lambda, based on the amino acid sequences of their constant domains. Accordingly, the antibodies provided herein can, in certain embodiments, contain a kappa light chain constant domain. The antibodies provided herein can, in certain embodiments, also contain a lambda light chain constant domain. According to particular embodiments, the antibodies provided herein include heavy and/or light chain constant regions from rat or human antibodies. In specific embodiments, the contant region is a human constant region.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH 3-sheet framework, or one of three hypervariable regions (LCDR1, LCDR2 or LCDR3) within the non-framework region of the antibody VL 3-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the tables and/or Examples provided below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | | | | | | |
|---|---|---|---|---|---|---|---|
| | (Kabat + | | | | | | |
| | Chothia) | IMGT | Kabat | AbM | Chothia | Contact | Xencor |
| V_{H} CDR1 | 26-35 | 27-38 | 31-35 | 26-35 | 26-32 | 30-35 | 27-35 |
| V_{H} CDR2 | 50-65 | 56-65 | 50-65 | 50-58 | 53-55 | 47-58 | 54-61 |
| V_{H} CDR3 | 95-102 | 105-117 | 95-102 | 95-102 | 96-101 | 93-101 | 103-116 |
| V_{L} CDR1 | 24-34 | 27-38 | 24-34 | 24-34 | 26-32 | 30-36 | 27-38 |
| V_{L} CDR2 | 50-56 | 56-65 | 50-56 | 50-56 | 50-52 | 46-55 | 56-62 |
| V_{L} CDR3 | 89-97 | 105-117 | 89-97 | 89-97 | 91-96 | 89-96 | 97-105 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “antigen binding domain” as used herein refer to a set of six CDRs that, when present as part of a polypeptide sequence, specifically binds a target antigen as discussed herein. Thus, a “checkpoint antigen binding domain” binds a target checkpoint antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the VH CDR1, VH CDR2 and VH CDR3) and the variable light domain (vl or VL; containing the VL CDR1, VL CDR2 and VL CDR3), with the C-terminus of the VH domain being attached to the N-terminus of the CH1 domain of the heavy chain and the C-terminus of the VL domain being attached to the N-terminus of the constant light domain (and thus forming the light chain). In a scFv format, the VH and VL domains are covalently attached, generally through the use of a linker (a “scFv linker”) as outlined herein, into a single polypeptide sequence, which can be either (starting from the N-terminus) VH-linker-VL or VL-linker-VH, with the former being generally preferred (including optional domain linkers on each side, depending on the format used.

In general, the C-terminus of the scFv domain is attached to the N-terminus of the hinge in the second monomer.

As used herein, the term an “isolated antibody” refers to an antibody which is substantially free of other antibodies having different antigenic specificities (e.g., an isolated antibody that specifically binds to a given target is substantially free of antibodies that do not bind to that same target). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies provided herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fv segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, “Fab” or “Fab region” refers to the polypeptide that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody provided herein. In the context of a Fab, the Fab comprises an Fv region in addition to the CH1 and CL domains.

As used herein, “Fv,” “Fv fragment,” or “Fv region” refer to a polypeptide that comprises the VL and VH domains of an ABD. Fv regions can be formatted as both Fabs (as discussed above, generally two different polypeptides that also include the constant regions as outlined above) and scFvs, where the VL and VH domains are combined (generally with a linker as discussed herein) to form an scFv.

The term “single chain Fv” or “scFv” refers to a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH). The order of the VH and VL domain can be indicated in the name, e.g., H.X_L.Y means N- to C-terminal is VH-linker-VL, and L.Y_H.X is VL-linker-VH.

The terms “Fc,” “Fc region,” or “Fc domain” as used herein refer to the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.) Human IgG Fc domains are of particular use in the methods provided herein, and can be the Fc domain from human IgG1, IgG2 or IgG4.

As used herein, “heavy chain constant region” refers to the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

As used herein, “variable region” or “variable domain” refers to the region of an immunoglobulin that comprises one or more Ig domains substantially encoded by any of the Vx, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein).

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope.

Provided herein are a number of antibody domains that have sequence identity to human antibody domains. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Sequence identity between two similar sequences (e.g., antibody variable domains) can be measured by algorithms such as that of Smith, T. F. & Waterman, M. S. (1981) “Comparison of Biosequences,” Adv. Appl. Math. 2:482 [local homology algorithm]; Needleman, S. B. & Wunsch, CD. (1970) “A General Method Applicable to the Search for Similarities in the Amino Acid Sequence of Two Proteins,” J. Mol. Biol. 48:443 [homology alignment algorithm], Pearson, W. R. & Lipman, D. J. (1988) “Improved Tools for Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see, e.g., blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

The antibodies provided herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the terms “treat,” “treatment,” and “treating” refer to the reduction or amelioration or elimination of the progression, severity and/or effect associated with a solid malignant tumor described herein, or the improvement in the solid malignant tumor condition, or the improvement in the disease associated with the solid malignant tumor, or the increase in the immune system response of the human subject, or the amelioration of one or more symptoms (preferably, one or more discernible symptoms) of a solid malignant tumor described herein resulting from the administration of one or more therapies. In specific embodiments, the terms “treat,” “treatment,” and “treating” refer to the amelioration of at least one measurable physical parameter of a solid malignant tumor described herein, such as tumor size, rate of tumor growth, number of tumor cells, tumor invasiveness, presence of metastasis, or extent of metastasis. In other embodiments the terms “treat,” “treatment,” and “treating” refer to the inhibition of the progression of a solid malignant tumor described herein, either physically by, e.g., stabilization of a discernible symptom, physiologically by, e.g., stabilization of a physical parameter, or both. In other embodiments the terms “treat,” “treatment,” and “treating” refer to an increase in the immune system response of the human subject, such as increased T cell infiltration, increased T cell activation, upregulation of IFN pathways, upregulation of antigen presentation pathway, or increased Ki67+ induction in T cells following treatment with pembrolizumab or nivolumab. In an exemplary embodiment, treating a solid malignant tumor provides an improvement, or a lack of progression, in the disease associated with the tumor or the tumor condition, and/or an improvement, or a lack of progression, in the symptoms associated with the disease or condition. For example, treating a solid malignant tumor refers to one or more of the following: (1) a reduction in the number of solid malignant tumor cells; (2) an increase in solid malignant tumor cell death; (3) inhibition of solid malignant tumor cell survival; (5) inhibition (i.e., slowing to some extent, preferably lack of progression) of solid malignant tumor growth, such as stable disease; (6) inhibition of solid malignant tumor cell metastasis; (7) an increase in progression-free survival; (8) an increase in overall survival rate; and (9) some relief from one or more symptoms associated with the disease or condition. Additional descriptions regarding treatment can be found in the RECIST criteria (Eisenhauer et al. (2009) Eur J Cancer. 45:228-47; Chalian et al. (2011) Radiographics 31:2093-105); the imRECIST criteria (Hodi et al. (2018) J Clin Oncol. 36:850-858); the modified IrRC criteria (Wolchok et al. (2009) Clin Cancer Res. 15:7412-20); and the PCWG3 criteria (Scher, et al. (2016) Clin Oncol. 34:1402-18). In some embodiments, treating a solid malignant tumor involves administering the bispecific antibody for a pre-specified period of time, discontinuing administration for another specific period of time, and resuming administration of the bispecific antibody for yet another specific period of time. In some embodiments, treating a solid malignant tumor involves administering the bispecific antibody until one of the treatment effects described herein is achieved, pausing administration of the bispecific antibody while this treatment effect continues to be observed, and resuming administration of the bispecific antibody if this treatment effect ceases to be observed.

Solid malignant tumor treatment can be determined by standardized response criteria specific to the disease associated with the tumor or the tumor condition. Solid malignant tumor response can be assessed for changes in tumor morphology (i.e., with neo-adjuvant use of a therapy, such as assessment of pathological response) or tumor metrics (i.e., overall tumor burden, tumor size, and the like) using screening techniques such as magnetic resonance imaging (MRI) scan, positron emission tomography (PET) scan, x-radiographic imaging, radionuclide scan, computed tomographic (CT) scan, bone scan imaging, endoscopy, tumor sampling including bone marrow aspiration (BMA), and counting of tumor marker levels and/or tumor cells in the circulation.

Treatment according to the methods provided herein includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

The terms “patient,” “subject,” and “human subject” are used interchangeably herein. As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc. In a specific embodiment, the subject is a human.

### 4.2 Overview

Provided herein, in certain aspects, methods for treating lymphoma using a combination of a CD19 antibody, an anti-CD3×anti-CD20 multispecific antibody, and a compound having Compound A. The methods provided herein are particularly useful in the treatment of Diffuse Large Cell Lymphoma (DLBCL), including relapsed or refractory DLBCL.

### 4.3 Pharmaceutical Compositions

### Antibodies

Provided herein are methods of treating lymphoma using a combination therapy. In certain embodiments, the combination therapy comprises a multispecific antibody, wherein the multispecific antibody comprises a first binding domain that binds to CD3 and a second binding domain that binds to CD20 (“CD3×CD20 antibody”), and a CD19 antibody. In a specific embodiment, the combination therapy further comprises Compound A. In other embodiments, the combination therapy further comprises a pharmaceutically acceptable salt, solvate, or stereoisomer of Compound A. Other compounds useful in the methods provided herein are compounds (e.g., Compound A) that are racemic, stereomerically enriched or stereomerically pure, and pharmaceutically acceptable salts, solvates, hydrates, stereoisomers, clathrates, and prodrugs thereof.

Amino acid sequences of exemplary CD19 antibodies and CD3×CD23 multispecific antibodies useful in the methods and combination therapies provided herein are shown in Table 1 below.

TABLE 1
Amino Acid Sequences of Exemplary CD19 and CD3xCD20Antibodies

| Description | Sequence | SEQ ID NO |
|---|---|---|
| Anti-CD19 VH CDR1 | SYVMH | 1 |
| Anti-CD19 VH CDR2 | YINPYNDGTKYNEKFQG | 2 |
| Anti-CD19 VH CDR3 | GTYYYGTRVFDY | 3 |
| Anti-CD19 VL CDR1 | RSSKSLQNVNGNTYLY | 4 |
| Anti-CD19 VL CDR2 | RMSNLNS | 5 |
| Anti-CD19 VL CDR3 | MQHLEYPIT | 6 |
| Anti-CD3 VH CDR1 | TYAMN | 7 |
| Anti-CD3 VH CDR2 | RIRSKYNNYATYYADSVKG | 8 |
| Anti-CD3 VH CDR3 | HGNFGDSYVSWFAY | 9 |
| Anti-CD3 VL CDR1 | GSSTGAVTTSNYAN | 10 |
| Anti-CD3 VL CDR2 | GTNKRAP | 11 |
| Anti-CD3 VL CDR3 | ALWYSNHWV | 12 |
| Anti-CD20 VH CDR1 | SYNMH | 13 |
| Anti-CD20 VH CDR2 | AIYPGNGDTSYNQKFQG | 14 |
| Anti-CD20 VH CDR3 | STYYGGDWYFNV | 15 |
| Anti-CD20 VL CDR1 | RASSSVSYIH | 16 |
| Anti-CD20 VL CDR2 | ATSNLAS | 17 |
| Anti-CD20 VL CDR3 | QQWTSNPPT | 18 |
| Anti-CD19 VH | EVQLVESGGGLVKPGGSLKLSCAASGYTFTSYVMH | 19 |
| | WVRQAPGKGLEWIGYINPYNDGTKYNEKFQGRVTI | |
| | SSDKSISTAYMELSSLRSEDTAMYYCARGTYYYGT | |
| | RVFDYWGQGTLVTVSS | |
| Anti-CD19 VL | DIVMTQSPATLSLSPGERATLSCRSSKSLQNVNGN | 20 |
| | TYLYWFQQKPGQSPQLLIYRMSNLNSGVPDRFSGS | |
| | GSGTEFTLTISSLEPEDFAVYYCMQHLEYPITFGA | |
| | GTKLEIK | |
| Anti-CD3 VH | EVQLVESGGGLVQPGGSLRLSCAASGFTFSTYAMN | 21 |
| | WVRQAPGKGLEWVGRIRSKYNNYATYYADSVKGRF | |
| | TISRDDSKNTLYLQMNSLRAEDTAVYYCVRHGNFG | |
| | DSYVSWFAYWGQGTLVTVSS | |
| Anti-CD3 VL | QAVVTQEPSLTVSPGGTVTLTCGSSTGAVTTSNYA | 22 |
| | NWVQQKPGKSPRGLIGGTNKRAPGVPARFSGSLLG | |
| | GKAALTISGAQPEDEADYYCALWYSNHWVFGGGTK | |
| | LTVL | |
| Anti-CD20 VH | QVQLVQSGAEVKKPGASVKVSCKASGYTFTSYNMH | 23 |
| | WVRQAPGQGLEWMGAIYPGNGDTSYNQKFQGRVTI | |
| | TADKSISTAYMELSSLRSEDTAVYYCARSTYYGGD | |
| | WYFNVWGAGTLVTVSS | |
| Anti-CD20 VL | QIVLTQSPSSLSASVGDRVTITCRASSSVSYIHWF | 24 |
| | QQKPGKSPKPLIYATSNLASGVPVRFSGSGSGTDY | |
| | TLTISSLQPEDFATYYCQQWTSNPPTFGGGTKVEI | |
| | K | |
| scFv-linker-CH2-CH3 | EVQLVESGGGLVQPGGSLRLSCAASGFTFSTYAMN | 25 |
| | WVRQAPGKGLEWVGRIRSKYNNYATYYADSVKGRF | |
| | TISRDDSKNTLYLQMNSLRAEDTAVYYCVRHGNFG | |
| | DSYVSWFAYWGQGTLVTVSSGKPGSGKPGSGKPGS | |
| | GKPGSQAVVTQEPSLTVSPGGTVTLTCGSSTGAVT | |
| | TSNYANWVQQKPGKSPRGLIGGTNKRAPGVPARFS | |
| | GSLLGGKAALTISGAQPEDEADYYCALWYSNHWVF | |
| | GGGTKLTVLEPKSSDKTHTCPPCPAPPVAGPSVFL | |
| | FPPKPKDTLMISRTPEVTCVVVDVKHEDPEVKFNW | |
| | YVDGVEVHNAKTKPREEQYNSTYRVVSVLTVLHQD | |
| | WLNGKEYKCKVSNKALPAPIEKTISKAKGQPREPQ | |
| | VYTLPPSREQMTKNQVKLTCLVKGFYPSDIAVEWE | |
| | SNGQPENNYKTTPPVLDSDGSFFLYSKLTVDKSRW | |
| | QQGNVFSCSVMHEALHNHYTQKSLSLSPGK | |
| VH-CH1-hinge-CH2- | QVQLVQSGAEVKKPGASVKVSCKASGYTFTSYNMH | 26 |
| CH3 | WVRQAPGQGLEWMGAIYPGNGDTSYNQKFQGRVTI | |
| | TADKSISTAYMELSSLRSEDTAVYYCARSTYYGGD | |
| | WYFNVWGAGTLVTVSSASTKGPSVFPLAPSSKSTS | |
| | GGTAALGCLVKDYFPEPVTVSWNSGALTSGVHTFP | |
| | AVLQSSGLYSLSSVVTVPSSSLGTQTYICNVNHKP | |
| | SDTKVDKKVEPKSCDKTHTCPPCPAPPVAGPSVFL | |
| | FPPKPKDTLMISRTPEVTCVVVDVKHEDPEVKFNW | |
| | YVDGVEVHNAKTKPREEEYNSTYRVVSVLTVLHQD | |
| | WLNGKEYKCKVSNKALPAPIEKTISKAKGQPREPQ | |
| | VYTLPPSREEMTKNQVSLTCDVSGFYPSDIAVEWE | |
| | SDGQPENNYKTTPPVLDSDGSFFLYSKLTVDKSRW | |
| | EQGDVFSCSVMHEALHNHYTQKSLSLSPGK | |
| VL-CL | QIVLTQSPSSLSASVGDRVTITCRASSSVSYIHWF | 27 |
| | QQKPGKSPKPLIYATSNLASGVPVRFSGSGSGTDY | |
| | TLTISSLQPEDFATYYCQQWTSNPPTFGGGTKVEI | |
| | KRTVAAPSVFIFPPSDEQLKSGTASVVCLLNNFYP | |
| | REAKVQWKVDNALQSGNSQESVTEQDSKDSTYSLS | |
| | STLTLSKADYEKHKVYACEVTHQGLSSPVTKSFNR | |
| | GEC | |
| Anti-CD 19 heavy | EVQLVESGGGLVKPGGSLKLSCAASGYTFTSYVMH | 28 |
| chain | WVRQAPGKGLEWIGYINPYNDGTKYNEKFQGRVTI | |
| | SSDKSISTAYMELSSLRSEDTAMYYCARGTYYYGT | |
| | RVFDYWGQGTLVTVSSASTKGPSVFPLAPSSKSTS | |
| | GGTAALGCLVKDYFPEPVTVSWNSGALTSGVHTFP | |
| | AVLQSSGLYSLSSVVTVPSSSLGTQTYICNVNHKP | |
| | SNTKVDKKVEPKSCDKTHTCPPCPAPELLGGPDVF | |
| | LFPPKPKDTLMISRTPEVTCVVVDVSHEDPEVQFN | |
| | WYVDGVEVHNAKTKPREEQFNSTFRVVSVLTVVHQ | |
| | DWLNGKEYKCKVSNKALPAPEEKTISKTKGQPREP | |
| | QVYTLPPSREEMTKNQVSLTCLVKGFYPSDIAVEW | |
| | ESNGQPENNYKTTPPMLDSDGSFFLYSKLTVDKSR | |
| | WQQGNVFSCSVMHEALHNHYTQKSLSLSPGK | |
| Anti-CD19 | DIVMTQSPATLSLSPGERATLSCRSSKSLQNVNGN | 29 |
| light chain | TYLYWFQQKPGQSPQLLIYRMSNLNSGVPDRFSGS | |
| | GSGTEFTLTISSLEPEDFAVYYCMQHLEYPITFGA | |
| | GTKLEIKRTVAAPSVFIFPPSDEQLKSGTASVVCL | |
| | LNNFYPREAKVQWKVDNALQSGNSQESVTEQDSKD | |
| | STYSLSSTLTLSKADYEKHKVYACEVTHQGLSSPV | |
| | TKSFNRGEC | |
| mosunetuzumab | EVQLVQSGAEVKKPGASVKVSCKASGYTFTNYYIH | 30 |
| anti-CD3e heavy | WVRQAPGQGLEWIGWIYPGDGNTKYNEKEKGRATL | |
| chain | TADTSTSTAYLELSSLRSEDTAVYYCARDSYSNYY | |
| | FDYWGQGTLVTVSSASTKGPSVEPLAPSSKSTSGG | |
| | TAALGCLVKDYFPEPVTVSWNSGALTSGVHTFPAV | |
| | LQSSGLYSLSSVVTVPSSSLGTQTYICNVNHKPSN | |
| | TKVDKKVEPKSCDKTHTCPPCPAPELLGGPSVFLF | |
| | PPKPKDTLMISRTPEVTCVVVDVSHEDPEVKFNWY | |
| | VDGVEVHNAKTKPREEQYGSTYRVVSVLTVLHQDW | |
| | LNGKEYKCKVSNKALPAPIEKTISKAKGQPREPQV | |
| | YTLPPSREEMTKNQVSLSCAVKGFYPSDIAVEWES | |
| | NGQPENNYKTTPPVLDSDGSFFLVSKLTVDKSRWQ | |
| | QGNVFSCSVMHEALHNHYTQKSLSLSPGK | |
| mosunetuzumab | DIVMTQSPDSLAVSLGERATINCKSSQSLLNSRTR | 31 |
| anti-CD3e light chain | KNYLAWYQQKPGQPPKLLIYWASTRESGVPDRFSG | |
| | SGSGTDETLTISSLQAEDVAVYYCTQSFILRTFGQ | |
| | GTKVEIKRTVAAPSVFIFPPSDEQLKSGTASVVCL | |
| | LNNFYPREAKVQWKVDNALQSGNSQESVTEQDSKD | |
| | STYSLSSTLTLSKADYEKHKVYACEVTHQGLSSPV | |
| | TKSFNRGEC | |
| mosunetuzumab | EVQLVESGGGLVQPGGSLRLSCAASGYTFTSYNMH | 32 |
| anti-CD20 heavy | WVRQAPGKGLEWVGAIYPGNGDTSYNQKEKGRFTI | |
| chain | SVDKSKNTLYLQMNSLRAEDTAVYYCARVVYYSNS | |
| | YWYFDVWGQGTLVTVSSASTKGPSVFPLAPSSKST | |
| | SGGTAALGCLVKDYFPEPVTVSWNSGALTSGVHTF | |
| | PAVLQSSGLYSLSSVVTVPSSSLGTQTYICNVNHK | |
| | PSNTKVDKKVEPKSCDKTHTCPPCPAPELLGGPSV | |
| | FLFPPKPKDTLMISRTPEVTCVVVDVSHEDPEVKF | |
| | NWYVDGVEVHNAKTKPREEQYGSTYRVVSVLTVLH | |
| | QDWLNGKEYKCKVSNKALPAPIEKTISKAKGQPRE | |
| | PQVYTLPPSREEMTKNQVSLWCLVKGFYPSDIAVE | |
| | WESNGQPENNYKTTPPVLDSDGSFFLYSKLTVDKS | |
| | RWQQGNVFSCSVMHEALHNHYTQKSLSLSPGK | |
| mosunetuzumab | DIQMTQSPSSLSASVGDRVTITCRASSSVSYMHWY | 33 |
| anti-CD20 light chain | QQKPGKAPKPLIYAPSNLASGVPSRFSGSGSGTDF | |
| | TLTISSLQPEDFATYYCQQWSENPPTFGQGTKVEI | |
| | KRTVAAPSVFIFPPSDEQLKSGTASVVCLLNNFYP | |
| | REAKVQWKVDNALQSGNSQESVTEQDSKDSTYSLS | |
| | STLTLSKADYEKHKVYACEVTHQGLSSPVTKSFNR | |
| | GEC | |
| epcoritamab | EVKLVESGGGLVQPGGSLRLSCAASGFTFNTYAMN | 34 |
| anti-CD3e heavy | WVRQAPGKGLEWVARIRSKYNNYATYYADSVKDRF | |
| chain | TISRDDSKSSLYLQMNNLKTEDTAMYYCVRHGNFG | |
| | NSYVSWFAYWGQGTLVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPEFEGG | |
| | PSVFLFPPKPKDTLMISRTPEVTCVVVAVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFLLYSKLTV | |
| | DKSRWQQGNVFSCSVMHEALHNHYTQKSLSLSPG | |
| epcoritamab | QAVVTQEPSFSVSPGGTVTLTCRSSTGAVTTSNYA | 35 |
| anti-CD3e light chain | NWVQQTPGQAFRGLIGGTNKRAPGVPARFSGSLIG | |
| | DKAALTITGAQADDESIYFCALWYSNLWVFGGGTK | |
| | LTVLGQPKAAPSVTLFPPSSEELQANKATLVCLIS | |
| | DFYPGAVTVAWKADSSPVKAGVETTTPSKQSNNKY | |
| | AASSYLSLTPEQWKSHRSYSCQVTHEGSTVEKTVA | |
| | PTECS | |
| epcoritamab | EVQLVESGGGLVQPDRSLRLSCAASGFTFHDYAMH | 36 |
| anti-CD20 heavy | WVRQAPGKGLEWVSTISWNSGTIGYADSVKGRFTI | |
| chain | SRDNAKNSLYLQMNSLRAEDTALYYCAKDIQYGNY | |
| | YYGMDVWGQGTTVTVSSASTKGPSVFPLAPSSKST | |
| | SGGTAALGCLVKDYFPEPVTVSWNSGALTSGVHTF | |
| | PAVLQSSGLYSLSSVVTVPSSSLGTQTYICNVNHK | |
| | PSNTKVDKRVEPKSCDKTHTCPPCPAPEFEGGPSV | |
| | FLFPPKPKDTLMISRTPEVTCVVVAVSHEDPEVKF | |
| | NWYVDGVEVHNAKTKPREEQYNSTYRVVSVLTVLH | |
| | QDWLNGKEYKCKVSNKALPAPIEKTISKAKGQPRE | |
| | PQVYTLPPSREEMTKNQVSLTCLVKGFYPSDIAVE | |
| | WESNGQPENNYKTTPPVLDSDGSFFLYSRLTVDKS | |
| | RWQQGNVFSCSVMHEALHNHYTQKSLSLSPG | |
| epcoritamab | EIVLTQSPATLSLSPGERATLSCRASQSVSSYLAW | 37 |
| anti-CD20 light chain | YQQKPGQAPRLLIYDASNRATGIPARFSGSGSGTD | |
| | FTLTISSLEPEDFAVYYCQQRSNWPITFGQGTRLE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| odronextamab | EVQLVESGGGLVQPGRSLRLSCAASGFTFDDYTMH | 38 |
| anti-CD3 heavy chain | WVRQAPGKGLEWVSGISWNSGSIGYADSVKGRFTI | |
| | SRDNAKKSLYLQMNSLRAEDTALYYCAKDNSGYGH | |
| | YYYGMDVWGQGTTVTVASASTKGPSVFPLAPCSRS | |
| | TSESTAALGCLVKDYFPEPVTVSWNSGALTSGVHT | |
| | FPAVLQSSGLYSLSSVVTVPSSSLGTKTYTCNVDH | |
| | KPSNTKVDKRVESKYGPPCPPCPAPPVAGPSVFLF | |
| | PPKPKDTLMISRTPEVTCVVVDVSQEDPEVQFNWY | |
| | VDGVEVHNAKTKPREEQFNSTYRVVSVLTVLHQDW | |
| | LNGKEYKCKVSNKGLPSSIEKTISKAKGQPREPQV | |
| | YTLPPSQEEMTKNQVSLTCLVKGFYPSDIAVEWES | |
| | NGQPENNYKTTPPVLDSDGSFFLYSRLTVDKSRWQ | |
| | EGNVFSCSVMHEALHNRFTQKSLSLSLGK | |
| odronextamab | EIVMTQSPATLSVSPGERATLSCRASQSVSSNLAW | 39 |
| light chain | YQQKPGQAPRLLIYGASTRATGIPARFSGSGSGTE | |
| | FTLTISSLQSEDFAVYYCQHYINWPLTFGGGTKVE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| odronextamab anti- | EVQLVESGGGLVQPGRSLRLSCVASGFTFNDYAMH | 40 |
| CD20 heavy chain | WVRQAPGKGLEWVSVISWNSDSIGYADSVKGRFTI | |
| | SRDNAKNSLYLQMHSLRAEDTALYYCAKDNHYGSG | |
| | SYYYYQYGMDVWGQGTTVTVSSASTKGPSVFPLAP | |
| | CSRSTSESTAALGCLVKDYFPEPVTVSWNSGALTS | |
| | GVHTFPAVLQSSGLYSLSSVVTVPSSSLGTKTYTC | |
| | NVDHKPSNTKVDKRVESKYGPPCPPCPAPPVAGPS | |
| | VFLFP | |
| | PKPKDTLMISRTPEVTCVVVDVSQEDPEVQFNWYV | |
| | DGVEVHNAKTKPREEQFNSTYRVVSVLTVLHQDWL | |
| | NGKEYKCKVSNKGLPSSIEKTISKAKGQPREPQVY | |
| | TLPPSQEEMTKNQVSLTCLVKGFYPSDIAVEWESN | |
| | GQPENNYKTTPPVLDSDGSFFLYSRLTVDKSRWQE | |
| | GNVFSCSVMHEALHNHYTQKSLSLSLGK | |
| glofitamab | QVQLVQSGAEVKKPGSSVKVSCKASGYAFSYSWIN | 41 |
| anti-CD20/CD3 heavy | WVRQAPGQGLEWMGRIFPGDGDTDYNGKFKGRVTI | |
| chain | TADKSTSTAYMELSSLRSEDTAVYYCARNVFDGYW | |
| | LVYWGQGTLVTVSSASTKGPSVFPLAPSSKSTSGG | |
| | TAALGCLVEDYFPEPVTVSWNSGALTSGVHTFPAV | |
| | LQSSGLYSLSSVVTVPSSSLGTQTYICNVNHKPSN | |
| | TKVDEKVEPKSCDGGGGSGGGGSQAVVTQEPSLTV | |
| | SPGGTVTLTCGSSTGAVTTSNYANWVQEKPGQAFR | |
| | GLIGGTNKRAPGTPARFSGSLLGGKAALTLSGAQP | |
| | EDEAEYYCALWYSNLWVFGGGTKLTVLSSASTKGP | |
| | SVFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKKVEPKSCDKTHTCPP | |
| | CPAPEAAGGPSVFLFPPKPKDTLMISRTPEVTCVV | |
| | VDVSHEDPEVKFNWYVDGVEVHNAKTKPREEQYNS | |
| | TYRVVSVLTVLHQDWLNGKEYKCKVSNKALGAPIE | |
| | KTISKAKGQPREPQVYTLPPCRDELTKNQVSLWCL | |
| | VKGFYPSDIAVEWESNGQPENNYKTTPPVLDSDGS | |
| | FFLYSKLTVDKSRWQQGNVFSCSVMHEALHNHYTQ | |
| | KSLSLSPGK | |
| glofitamab | EVQLLESGGGLVQPGGSLRLSCAASGFTFSTYAMN | 42 |
| anti-CD3e light chain | WVRQAPGKGLEWVSRIRSKYNNYATYYADSVKGRF | |
| | TISRDDSKNTLYLQMNSLRAEDTAVYYCVRHGNFG | |
| | NSYVSWFAYWGQGTLVTVSSASVAAPSVFIFPPSD | |
| | EQLKSGTASVVCLLNNFYPREAKVQWKVDNALQSG | |
| | NSQESVTEQDSKDSTYSLSSTLTLSKADYEKHKVY | |
| | ACEVTHQGLSSPVTKSFNRGEC | |
| glofitamab | QVQLVQSGAEVKKPGSSVKVSCKASGYAFSYSWIN | 43 |
| anti-CD20 heavy | WVRQAPGQGLEWMGRIFPGDGDTDYNGKFKGRVTI | |
| chain | TADKSTSTAYMELSSLRSEDTAVYYCARNVFDGYW | |
| | LVYWGQGTLVTVSSASTKGPSVFPLAPSSKSTSGG | |
| | TAALGCLVEDYFPEPVTVSWNSGALTSGVHTFPAV | |
| | LQSSGLYSLSSVVTVPSSSLGTQTYICNVNHKPSN | |
| | TKVDEKVEPKSCDKTHTCPPCPAPEAAGGPSVFLF | |
| | PPKPKDTLMISRTPEVTCVVVDVSHEDPEVKFNWY | |
| | VDGVEVHNAKTKPREEQYNSTYRVVSVLTVLHQDW | |
| | LNGKEYKCKVSNKALGAPIEKTISKAKGQPREPQV | |
| | CTLPPSRDELTKNQVSLSCAVKGFYPSDIAVEWES | |
| | NGQPENNYKTTPPVLDSDGSFFLVSKLTVDKSRWQ | |
| | QGNVFSCSVMHEALHNHYTQKSLLSPGK | |
| glofitamab | DIVMTQTPLSLPVTPGEPASISCRSSKSLLHSNGI | 44 |
| anti-CD20 light chain | TYLYWYLQKPGQSPQLLIYQMSNLVSGVPDRFSGS | |
| | GSGTDFTLKISRVEAEDVGVYYCAQNLELPYTFGG | |
| | GTKVEIKRTVAAPSVFIFPPSDRKLKSGTASVVCL | |
| | LNNFYPREAKVQWKVDNALQSGNSQESVTEQDSKD | |
| | STYSLSSTLTLSKADYEKHKVYACEVTHQGLSSPV | |
| | TKSFNRGEC | |

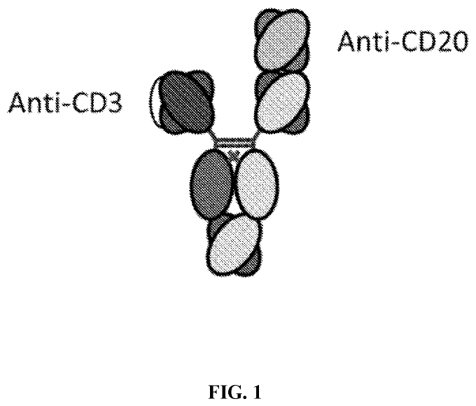

In some embodiments, the CD3×CD20 antibody, has a “bottle opener” format (also referred to as the “triple F” format) as is generally depicted in FIG. 1. In this embodiment, the CD3 antigen binding domain is the scFv in the bottle opener format and the CD20 antigen binding domain is the Fab in the bottle opener format.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1.

In one embodiment, the CD3×CD20 antibody comprises a first binding domain that binds to CD3, wherein the first binding domain comprises a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the CD3×CD20 antibody comprises a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1.

In one embodiment, the CD3×CD20 antibody comprises a second binding domain that binds to CD20, wherein the second binding domain comprises a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the CD3×CD20 antibody comprises a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1, and a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 21.

In one embodiment, the CD3×CD20 antibody comprises a first binding domain that binds to CD3, wherein the first binding domain comprises a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 22.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 21, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 22.

In one embodiment, the CD3×CD20 antibody comprises a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 23.

In one embodiment, the CD3×CD20 antibody comprises a second binding domain that binds to CD20, wherein the first binding domain comprises a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 24.

In one embodiment, the CD3×CD20 antibody comprises a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 23, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 24.

In one embodiment, the CD3×CD20 antibody comprises a first binding domain that binds to CD3, wherein the first binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 21, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 22, and a second binding domain that binds to CD20, wherein the second binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 23, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 24.

In one embodiment, the CD3×CD20 antibody comprises a first binding domain that binds to CD3, wherein the first binding domain comprises a VH domain comprising a VH CDR1, a VH CDR2, and a VH CDR3 having an amino acid sequence of SEQ ID NO:7, SEQ ID NO:8, and SEQ ID NO:9, respectively.

In one embodiment, the CD3×CD20 antibody comprises a first binding domain that binds to CD3, wherein the first binding domain comprises a VL domain comprising a VL CDR1, a VL CDR2, and a VL CDR3 having an amino acid sequence of SEQ ID NO:10, SEQ ID NO:11, and SEQ ID NO:12, respectively.

In one embodiment, the CD3×CD20 antibody comprises a first binding domain that binds to CD3, wherein the first binding domain comprises a VH domain comprising a VH CDR1, a VH CDR2, and a VH CDR3 having the amino acid sequence of SEQ ID NO:7, SEQ ID NO:8, and SEQ ID NO:9, respectively, and a VL domain comprising a VL CDR1, a VL CDR2, and a VL CDR3 having an amino acid sequence of SEQ ID NO:10, SEQ ID NO:11, and SEQ ID NO:12, respectively.

In one embodiment, the CD3×CD20 antibody comprises a second binding domain that binds to CD20, wherein the second binding domain comprises a VH domain comprising a VH CDR1, a VH CDR2, and a VH CDR3 having an amino acid sequence of SEQ ID NO:13, SEQ ID NO:14, and SEQ ID NO:15, respectively.

In one embodiment, the CD3×CD20 antibody comprises a second binding domain that binds to CD20, wherein the second binding domain comprises a VL domain comprising a VL CDR1, a VL CDR2, and a VL CDR3 having an amino acid sequence of SEQ ID NO:16, SEQ ID NO:17, and SEQ ID NO:18, respectively.

In one embodiment, the CD3×CD20 antibody comprises a second binding domain that binds to CD20, wherein the second binding domain comprises a VH domain comprising a VH CDR1, a VH CDR2, and a VH CDR3 having an amino acid sequence of SEQ ID NO:13, SEQ ID NO:14, and SEQ ID NO:15, respectively, and a VL domain comprising a VL CDR1, a VL CDR2, and a VL CDR3 having an amino acid sequence of SEQ ID NO:16, SEQ ID NO:17, and SEQ ID NO:18, respectively.

In one embodiment, the CD3×CD20 antibody comprises a first binding domain that binds to CD3 and a second binding domain that binds to CD20, wherein the first binding domain comprises a VH domain comprising a VH CDR1, a VH CDR2, and a VH CDR3 having an amino acid sequence of SEQ ID NO:7, SEQ ID NO:8, and SEQ ID NO:9, respectively, and a VL domain comprising a VL CDR1, a VL CDR2, and a VL CDR3 having an amino acid sequence of SEQ ID NO: 10, SEQ ID NO: 11, and SEQ ID NO:12, respectively, and the second binding domain comprises a VH domain comprising a VH CDR1, a VH CDR2, and a VH CDR3 having an amino acid sequence of SEQ ID NO:13, SEQ ID NO:14, and SEQ ID NO:15, respectively, and a VL domain comprising a VL CDR1, a VL CDR2, and a VL CDR3 having an amino acid sequence of SEQ ID NO:16, SEQ ID NO:17, and SEQ ID NO:18, respectively.

In one embodiment, the CD3×CD20 antibody comprises a first binding domain that binds to CD3, wherein the first binding domain comprises a VH domain having an amino acid sequence that is about 90%, 95%, 98%, 99%, or 100% identical to the amino acid sequence of SEQ ID NO:21. In one embodiment, the first binding domain comprises a VH domain having an amino acid sequence that is about 90% identical to the amino acid sequence of SEQ ID NO:21. In another embodiment, the first binding domain comprises a VH domain having an amino acid sequence that is about 95% identical to the amino acid sequence of SEQ ID NO:21. In another embodiment, the first binding domain comprises a VH domain having an amino acid sequence that is about 98% identical to the amino acid sequence of SEQ ID NO:21. In another embodiment, the first binding domain comprises a VH domain having an amino acid sequence that is about 99% identical to the amino acid sequence of SEQ ID NO:21. In one embodiment, the first binding domain comprises a VH domain having an amino acid sequence that is about 100% identical to the amino acid sequence of SEQ ID NO:21.

In one embodiment, the CD3×CD20 antibody comprises a first binding domain that binds to CD3, wherein the first binding domain comprises a VL domain having an amino acid sequence that is about 90%, 95%, 98%, 99%, or 100% identical to the amino acid sequence of SEQ ID NO:22. In one embodiment, the first binding domain comprises a VL domain having an amino acid sequence that is about 90% identical to the amino acid sequence of SEQ ID NO:22. In another embodiment, the first binding domain comprises a VL domain having an amino acid sequence that is about 95% identical to the amino acid sequence of SEQ ID NO:22. In another embodiment, the first binding domain comprises a VL domain having an amino acid sequence that is about 98% identical to the amino acid sequence of SEQ ID NO:22. In another embodiment, the first binding domain comprises a VL domain having an amino acid sequence that is about 99% identical to the amino acid sequence of SEQ ID NO:22. In another embodiment, the first binding domain comprises a VL domain having an amino acid sequence that is about 100% identical to the amino acid sequence of SEQ ID NO:22.

In one embodiment, the CD3×CD20 antibody comprises a first binding domain that binds to CD3, wherein the first binding domain comprises a VH domain having an amino acid sequence that is about 90%, 95%, 98%, 99%, or 100% identical to the amino acid sequence of SEQ ID NO:21 and a VL domain having an amino acid sequence that is about 90%, 95%, 98%, 99%, or 100% identical to the amino acid sequence of SEQ ID NO:22. In one embodiment, the first binding domain comprises a VH domain having an amino acid sequence that is about 90%, identical to the amino acid sequence of SEQ ID NO:21 and a VL domain having an amino acid sequence that is about 90% identical to the amino acid sequence of SEQ ID NO:22. In another embodiment, the first binding domain comprises a VH domain having an amino acid sequence that is about 95%, identical to the amino acid sequence of SEQ ID NO:21 and a VL domain having an amino acid sequence that is about 95% identical to the amino acid sequence of SEQ ID NO:22. In another embodiment, the first binding domain comprises a VH domain having an amino acid sequence that is about 98%, identical to the amino acid sequence of SEQ ID NO:21 and a VL domain having an amino acid sequence that is about 98% identical to the amino acid sequence of SEQ ID NO:22. In another embodiment, the first binding domain comprises a VH domain having an amino acid sequence that is about 99%, identical to the amino acid sequence of SEQ ID NO:21 and a VL domain having an amino acid sequence that is about 99% identical to the amino acid sequence of SEQ ID NO:22. In another embodiment, the first binding domain comprises a VH domain having an amino acid sequence that is about 100%, identical to the amino acid sequence of SEQ ID NO:21 and a VL domain having an amino acid sequence that is about 100% identical to the amino acid sequence of SEQ ID NO:22.

In one embodiment, the CD3×CD20 antibody comprises a second binding domain that binds to CD20, wherein the second binding domain comprises a VH domain having an amino acid sequence that is about 90%, 95%, 98%, 99%, or 100% identical to the amino acid sequence of SEQ ID NO:23. In one embodiment, the second binding domain comprises a VH domain having an amino acid sequence that is about 90% identical to the amino acid sequence of SEQ ID NO:23. In another embodiment, the second binding domain comprises a VH domain having an amino acid sequence that is about 95% identical to the amino acid sequence of SEQ ID NO:23. In another embodiment, the second binding domain comprises a VH domain having an amino acid sequence that is about 98% identical to the amino acid sequence of SEQ ID NO:23. In another embodiment, the second binding domain comprises a VH domain having an amino acid sequence that is about 99% identical to the amino acid sequence of SEQ ID NO:23. In another embodiment, the second binding domain comprises a VH domain having an amino acid sequence that is about 100% identical to the amino acid sequence of SEQ ID NO:23.

In one embodiment, the CD3×CD20 antibody comprises a second binding domain that binds to CD20, wherein the second binding domain comprises a VL domain having an amino acid sequence that is about 90%, 95%, 98%, 99%, or 100% identical to the amino acid sequence of SEQ ID NO:24. In one embodiment, the second binding domain comprises a VL domain having an amino acid sequence that is about 90% identical to the amino acid sequence of SEQ ID NO:24. In another embodiment, the second binding domain comprises a VL domain having an amino acid sequence that is about 95% identical to the amino acid sequence of SEQ ID NO:24. In another embodiment, the second binding domain comprises a VL domain having an amino acid sequence that is about 98% identical to the amino acid sequence of SEQ ID NO:24. In another embodiment, the second binding domain comprises a VL domain having an amino acid sequence that is about 99% identical to the amino acid sequence of SEQ ID NO:24. In another embodiment, the second binding domain comprises a VL domain having an amino acid sequence that is about 100% identical to the amino acid sequence of SEQ ID NO:24.

In one embodiment, the CD3×CD20 antibody comprises a second binding domain that binds to CD20, wherein the second binding domain comprises a VH domain having an amino acid sequence that is about 90%, 95%, 98%, 99%, or 100% identical to the amino acid sequence of SEQ ID NO:23 and a VL domain having an amino acid sequence that is about 90%, 95%, 98%, 99%, or 100% identical to the amino acid sequence of SEQ ID NO:24. In one embodiment, the second binding domain comprises a VH domain having an amino acid sequence that is about 90%, identical to the amino acid sequence of SEQ ID NO:23 and a VL domain having an amino acid sequence that is about 90% identical to the amino acid sequence of SEQ ID NO:24. In another embodiment, the second binding domain comprises a VH domain having an amino acid sequence that is about 95%, identical to the amino acid sequence of SEQ ID NO:23 and a VL domain having an amino acid sequence that is about 95% identical to the amino acid sequence of SEQ ID NO:24. In another embodiment, the second binding domain comprises a VH domain having an amino acid sequence that is about 98%, identical to the amino acid sequence of SEQ ID NO:23 and a VL domain having an amino acid sequence that is about 98% identical to the amino acid sequence of SEQ ID NO:24. In another embodiment, the second binding domain comprises a VH domain having an amino acid sequence that is about 99%, identical to the amino acid sequence of SEQ ID NO:23 and a VL domain having an amino acid sequence that is about 99% identical to the amino acid sequence of SEQ ID NO:24. In another embodiment, the second binding domain comprises a VH domain having an amino acid sequence that is about 100%, identical to the amino acid sequence of SEQ ID NO:23 and a VL domain having an amino acid sequence that is about 100% identical to the amino acid sequence of SEQ ID NO:24.

In one embodiment, the CD3×CD20 antibody comprises a first binding domain that binds to CD3 and a second binding domain that binds to CD20, wherein the first binding domain comprises a VH domain having an amino acid sequence that is about 90%, 95%, 98%, 99%, or 100% identical to the amino acid sequence of SEQ ID NO:21 and a VL domain having an amino acid sequence that is about 90%, 95%, 98%, 99%, or 100% identical to the amino acid sequence of SEQ ID NO:22, and the second binding domain comprises a VH domain having an amino acid sequence that is about 90%, 95%, 98%, 99%, or 100% identical to the amino acid sequence of SEQ ID NO:23 and a VL domain having an amino acid sequence that is about 90%, 95%, 98%, 99%, or 100% identical to the amino acid sequence of SEQ ID NO:24. In one embodiment, the first binding domain comprises a VH domain having an amino acid sequence that is about 90%, identical to the amino acid sequence of SEQ ID NO:21 and a VL domain having an amino acid sequence that is about 90% identical to the amino acid sequence of SEQ ID NO:22, and the second binding domain comprises a VH domain having an amino acid sequence that is about 90%, identical to the amino acid sequence of SEQ ID NO:23 and a VL domain having an amino acid sequence that is about 90% identical to the amino acid sequence of SEQ ID NO:24. In another embodiment, the first binding domain comprises a VH domain having an amino acid sequence that is about 95%, identical to the amino acid sequence of SEQ ID NO:21 and a VL domain having an amino acid sequence that is about 95% identical to the amino acid sequence of SEQ ID NO:22, and the second binding domain comprises a VH domain having an amino acid sequence that is about 95%, identical to the amino acid sequence of SEQ ID NO:23 and a VL domain having an amino acid sequence that is about 95% identical to the amino acid sequence of SEQ ID NO:24. In another embodiment, the first binding domain comprises a VH domain having an amino acid sequence that is about 98%, identical to the amino acid sequence of SEQ ID NO:21 and a VL domain having an amino acid sequence that is about 98% identical to the amino acid sequence of SEQ ID NO:22, and the second binding domain comprises a VH domain having an amino acid sequence that is about 98%, identical to the amino acid sequence of SEQ ID NO:23 and a VL domain having an amino acid sequence that is about 98% identical to the amino acid sequence of SEQ ID NO:24. In another embodiment, the first binding domain comprises a VH domain having an amino acid sequence that is about 99%, identical to the amino acid sequence of SEQ ID NO:21 and a VL domain having an amino acid sequence that is about 99% identical to the amino acid sequence of SEQ ID NO:22, and the second binding domain comprises a VH domain having an amino acid sequence that is about 99%, identical to the amino acid sequence of SEQ ID NO:23 and a VL domain having an amino acid sequence that is about 99% identical to the amino acid sequence of SEQ ID NO:24. In another embodiment, the first binding domain comprises a VH domain having an amino acid sequence that is about 100%, identical to the amino acid sequence of SEQ ID NO:21 and a VL domain having an amino acid sequence that is about 100% identical to the amino acid sequence of SEQ ID NO:22, and the second binding domain comprises a VH domain having an amino acid sequence that is about 100%, identical to the amino acid sequence of SEQ ID NO:23 and a VL domain having an amino acid sequence that is about 100% identical to the amino acid sequence of SEQ ID NO:24.

In one embodiment, the CD3×CD20 antibody comprises a first monomer comprising, from N- to C-terminus, a scFv-linker-CH2-CH3 having the amino acid sequence of SEQ ID NO:25. In another embodiment, the CD3×CD20 antibody comprises a second monomer comprising, from N- to C-terminus, a VH-CH1-hinge-CH2-CH3 having the amino acid sequence of SEQ ID NO:26. In another embodiment, the CD3×CD20 antibody comprises a third monomer comprising, from N- to C-terminus, a VL-CL having the amino acid sequence of SEQ ID NO:27. In another embodiment, the CD3×CD20 antibody comprises a first monomer comprising, from N- to C-terminus, a scFv-linker-CH2-CH3 having the amino acid sequence of SEQ ID NO:25, a second monomer comprising, from N- to C-terminus, a VH-CH1-hinge-CH2-CH3 having the amino acid sequence of SEQ ID NO:26, and a third monomer comprising, from N- to C-terminus, a VL-CL having the amino acid sequence of SEQ ID NO:27.

In a specific embodiment, the CD3×CD20 antibody is plamotamab (XmAb13676). In one embodiment, the CD3×CD20 antibody is a biosimilar of plamotamab. In one embodiment, the CD3×CD20 antibody is a bioequivalent of plamotamab. In one embodiment, the CD3×CD20 antibody comprises an amino acid sequence of a VH CDR1, VH CDR2, and VH CDR3 of the CD3 binding domain of plamotamab. In one embodiment, the CD3×CD20 antibody comprises an amino acid sequence of a VL CDR1, VL CDR2, and VL CDR3 of the CD3 binding domain of plamotamab. In one embodiment, the CD3×CD20 antibody comprises an amino acid sequence of a VH CDR1, VH CDR2, and VH CDR3 of the CD20 binding domain of plamotamab. In one embodiment, the CD3×CD20 antibody comprises an amino acid sequence of a VL CDR1, VL CDR2, and VL CDR3 of the CD20 binding domain of plamotamab. In one embodiment, the CD3×CD20 antibody comprises an amino acid sequence of a VH CDR1, VH CDR2, and VH CDR3 of the CD3 binding domain of plamotamab, and a VL CDR1, VL CDR2, and VL CDR3 of the CD3 binding domain of plamotamab. In one embodiment, the CD3×CD20 antibody comprises an amino acid sequence of a VH CDR1, VH CDR2, and VH CDR3 of the CD20 binding domain of plamotamab, and a VL CDR1, VL CDR2, and VL CDR3 of the CD20 binding domain of plamotamab. In one embodiment, the CD3×CD20 antibody comprises an amino acid sequence of a VH CDR1, VH CDR2, and VH CDR3 of the CD3 binding domain of plamotamab, a VL CDR1, VL CDR2, and VL CDR3 of the CD3 binding domain of plamotamab, a VH CDR1, VH CDR2, and VH CDR3 of the CD20 binding domain of plamotamab, and a VL CDR1, VL CDR2, and VL CDR3 of the CD20 binding domain of plamotamab. In one embodiment, the CD3×CD20 antibody comprises an amino acid sequence of a VH of the CD3 binding domain of plamotamab. In one embodiment, the CD3×CD20 antibody comprises an amino acid sequence of a VL of the CD3 binding domain of plamotamab. In one embodiment, the CD3×CD20 antibody comprises an amino acid sequence of a VH of the CD20 binding domain of plamotamab. In one embodiment, the CD3×CD20 antibody comprises an amino acid sequence of a VL of the CD20 binding domain of plamotamab. In one embodiment, the CD3×CD20 antibody comprises an amino acid sequence of a VH of the CD3 binding domain of plamotamab, and a VL of the CD3 binding domain of plamotamab. In one embodiment, the CD3×CD20 antibody comprises an amino acid sequence of a VH of the CD20 binding domain of plamotamab, and a VL of the CD20 binding domain of plamotamab. In one embodiment, the CD3×CD20 antibody comprises an amino acid sequence of a VH of the CD3 binding domain of plamotamab, a VL of the CD3 binding domain of plamotamab, a VH of the CD20 binding domain of plamotamab, and a VL of the CD20 binding domain of plamotamab.

In another embodiment, the CD3×CD20 antibody is mosunetuzumab. In one embodiment, the CD3×CD20 antibody is a biosimilar of mosunetuzumab. In one embodiment, the CD3×CD20 antibody is a bioequivalent of mosunetuzumab. In one embodiment, the CD3×CD20 antibody comprises an amino acid sequence of a VH CDR1, VH CDR2, and VH CDR3 of the CD3 binding domain of mosunetuzumab. In one embodiment, the CD3×CD20 antibody comprises an amino acid sequence of a VL CDR1, VL CDR2, and VL CDR3 of the CD3 binding domain of mosunetuzumab. In one embodiment, the CD3×CD20 antibody comprises an amino acid sequence of a VH CDR1, VH CDR2, and VH CDR3 of the CD20 binding domain of mosunetuzumab. In one embodiment, the CD3×CD20 antibody comprises an amino acid sequence of a VL CDR1, VL CDR2, and VL CDR3 of the CD20 binding domain of mosunetuzumab. In one embodiment, the CD3×CD20 antibody comprises an amino acid sequence of a VH CDR1, VH CDR2, and VH CDR3 of the CD3 binding domain of mosunetuzumab, and a VL CDR1, VL CDR2, and VL CDR3 of the CD3 binding domain of mosunetuzumab. In one embodiment, the CD3×CD20 antibody comprises an amino acid sequence of a VH CDR1, VH CDR2, and VH CDR3 of the CD20 binding domain of mosunetuzumab, and a VL CDR1, VL CDR2, and VL CDR3 of the CD20 binding domain of mosunetuzumab. In one embodiment, the CD3×CD20 antibody comprises an amino acid sequence of a VH CDR1, VH CDR2, and VH CDR3 of the CD3 binding domain of mosunetuzumab, a VL CDR1, VL CDR2, and VL CDR3 of the CD3 binding domain of mosunetuzumab, a VH CDR1, VH CDR2, and VH CDR3 of the CD20 binding domain of mosunetuzumab, and a VL CDR1, VL CDR2, and VL CDR3 of the CD20 binding domain of mosunetuzumab. In one embodiment, the CD3×CD20 antibody comprises an amino acid sequence of a VH of the CD3 binding domain of mosunetuzumab. In one embodiment, the CD3×CD20 antibody comprises an amino acid sequence of a VL of the CD3 binding domain of mosunetuzumab. In one embodiment, the CD3×CD20 antibody comprises an amino acid sequence of a VH of the CD20 binding domain of mosunetuzumab. In one embodiment, the CD3×CD20 antibody comprises an amino acid sequence of a VL of the CD20 binding domain of mosunetuzumab. In one embodiment, the CD3×CD20 antibody comprises an amino acid sequence of a VH of the CD3 binding domain of mosunetuzumab, and a VL of the CD3 binding domain of mosunetuzumab. In one embodiment, the CD3×CD20 antibody comprises an amino acid sequence of a VH of the CD20 binding domain of mosunetuzumab, and a VL of the CD20 binding domain of mosunetuzumab. In one embodiment, the CD3×CD20 antibody comprises an amino acid sequence of a VH of the CD3 binding domain of mosunetuzumab, a VL of the CD3 binding domain of mosunetuzumab, a VH of the CD20 binding domain of mosunetuzumab, and a VL of the CD20 binding domain of mosunetuzumab.

In another embodiment, the CD3×CD20 antibody is epcoritamab. In one embodiment, the CD3×CD20 antibody is a biosimilar of epcoritamab. In one embodiment, the CD3×CD20 antibody is a bioequivalent of epcoritamab. In one embodiment, the CD3×CD20 antibody comprises an amino acid sequence of a VH CDR1, VH CDR2, and VH CDR3 of the CD3 binding domain of epcoritamab. In one embodiment, the CD3×CD20 antibody comprises an amino acid sequence of a VL CDR1, VL CDR2, and VL CDR3 of the CD3 binding domain of epcoritamab. In one embodiment, the CD3×CD20 antibody comprises an amino acid sequence of a VH CDR1, VH CDR2, and VH CDR3 of the CD20 binding domain of epcoritamab. In one embodiment, the CD3×CD20 antibody comprises an amino acid sequence of a VL CDR1, VL CDR2, and VL CDR3 of the CD20 binding domain of epcoritamab. In one embodiment, the CD3×CD20 antibody comprises an amino acid sequence of a VH CDR1, VH CDR2, and VH CDR3 of the CD3 binding domain of epcoritamab, and a VL CDR1, VL CDR2, and VL CDR3 of the CD3 binding domain of epcoritamab. In one embodiment, the CD3×CD20 antibody comprises an amino acid sequence of a VH CDR1, VH CDR2, and VH CDR3 of the CD20 binding domain of epcoritamab, and a VL CDR1, VL CDR2, and VL CDR3 of the CD20 binding domain of epcoritamab. In one embodiment, the CD3×CD20 antibody comprises an amino acid sequence of a VH CDR1, VH CDR2, and VH CDR3 of the CD3 binding domain of epcoritamab, a VL CDR1, VL CDR2, and VL CDR3 of the CD3 binding domain of epcoritamab, a VH CDR1, VH CDR2, and VH CDR3 of the CD20 binding domain of epcoritamab, and a VL CDR1, VL CDR2, and VL CDR3 of the CD20 binding domain of epcoritamab. In one embodiment, the CD3×CD20 antibody comprises an amino acid sequence of a VH of the CD3 binding domain of epcoritamab. In one embodiment, the CD3×CD20 antibody comprises an amino acid sequence of a VL of the CD3 binding domain of epcoritamab. In one embodiment, the CD3×CD20 antibody comprises an amino acid sequence of a VH of the CD20 binding domain of epcoritamab. In one embodiment, the CD3×CD20 antibody comprises an amino acid sequence of a VL of the CD20 binding domain of epcoritamab. In one embodiment, the CD3×CD20 antibody comprises an amino acid sequence of a VH of the CD3 binding domain of epcoritamab, and a VL of the CD3 binding domain of epcoritamab. In one embodiment, the CD3×CD20 antibody comprises an amino acid sequence of a VH of the CD20 binding domain of epcoritamab, and a VL of the CD20 binding domain of epcoritamab. In one embodiment, the CD3×CD20 antibody comprises an amino acid sequence of a VH of the CD3 binding domain of epcoritamab, a VL of the CD3 binding domain of epcoritamab, a VH of the CD20 binding domain of epcoritamab, and a VL of the CD20 binding domain of epcoritamab.

In another embodiment, the CD3×CD20 antibody is odronextamab. In one embodiment, the CD3×CD20 antibody is a biosimilar of odronextamab. In one embodiment, the CD3×CD20 antibody is a bioequivalent of odronextamab. In one embodiment, the CD3×CD20 antibody comprises an amino acid sequence of a VH CDR1, VH CDR2, and VH CDR3 of the CD3 binding domain of odronextamab. In one embodiment, the CD3×CD20 antibody comprises an amino acid sequence of a VL CDR1, VL CDR2, and VL CDR3 of the CD3 binding domain of odronextamab. In one embodiment, the CD3×CD20 antibody comprises an amino acid sequence of a VH CDR1, VH CDR2, and VH CDR3 of the CD20 binding domain of odronextamab. In one embodiment, the CD3×CD20 antibody comprises an amino acid sequence of a VL CDR1, VL CDR2, and VL CDR3 of the CD20 binding domain of odronextamab. In one embodiment, the CD3×CD20 antibody comprises an amino acid sequence of a VH CDR1, VH CDR2, and VH CDR3 of the CD3 binding domain of odronextamab, and a VL CDR1, VL CDR2, and VL CDR3 of the CD3 binding domain of odronextamab. In one embodiment, the CD3×CD20 antibody comprises an amino acid sequence of a VH CDR1, VH CDR2, and VH CDR3 of the CD20 binding domain of odronextamab, and a VL CDR1, VL CDR2, and VL CDR3 of the CD20 binding domain of odronextamab. In one embodiment, the CD3×CD20 antibody comprises an amino acid sequence of a VH CDR1, VH CDR2, and VH CDR3 of the CD3 binding domain of odronextamab, a VL CDR1, VL CDR2, and VL CDR3 of the CD3 binding domain of odronextamab, a VH CDR1, VH CDR2, and VH CDR3 of the CD20 binding domain of odronextamab, and a VL CDR1, VL CDR2, and VL CDR3 of the CD20 binding domain of odronextamab. In one embodiment, the CD3×CD20 antibody comprises an amino acid sequence of a VH of the CD3 binding domain of odronextamab. In one embodiment, the CD3×CD20 antibody comprises an amino acid sequence of a VL of the CD3 binding domain of odronextamab. In one embodiment, the CD3×CD20 antibody comprises an amino acid sequence of a VH of the CD20 binding domain of odronextamab. In one embodiment, the CD3×CD20 antibody comprises an amino acid sequence of a VL of the CD20 binding domain of odronextamab. In one embodiment, the CD3×CD20 antibody comprises an amino acid sequence of a VH of the CD3 binding domain of odronextamab, and a VL of the CD3 binding domain of odronextamab. In one embodiment, the CD3×CD20 antibody comprises an amino acid sequence of a VH of the CD20 binding domain of odronextamab, and a VL of the CD20 binding domain of odronextamab. In one embodiment, the CD3×CD20 antibody comprises an amino acid sequence of a VH of the CD3 binding domain of odronextamab, a VL of the CD3 binding domain of odronextamab, a VH of the CD20 binding domain of odronextamab, and a VL of the CD20 binding domain of odronextamab.

In another embodiment, the CD3×CD20 antibody is glofitamab. In one embodiment, the CD3×CD20 antibody is a biosimilar of glofitamab. In one embodiment, the CD3×CD20 antibody is a bioequivalent of glofitamab. In one embodiment, the CD3×CD20 antibody comprises an amino acid sequence of a VH CDR1, VH CDR2, and VH CDR3 of the CD3 binding domain of glofitamab. In one embodiment, the CD3×CD20 antibody comprises an amino acid sequence of a VL CDR1, VL CDR2, and VL CDR3 of the CD3 binding domain of glofitamab. In one embodiment, the CD3×CD20 antibody comprises an amino acid sequence of a VH CDR1, VH CDR2, and VH CDR3 of the CD20 binding domain of glofitamab. In one embodiment, the CD3×CD20 antibody comprises an amino acid sequence of a VL CDR1, VL CDR2, and VL CDR3 of the CD20 binding domain of glofitamab. In one embodiment, the CD3×CD20 antibody comprises an amino acid sequence of a VH CDR1, VH CDR2, and VH CDR3 of the CD3 binding domain of glofitamab, and a VL CDR1, VL CDR2, and VL CDR3 of the CD3 binding domain of glofitamab. In one embodiment, the CD3×CD20 antibody comprises an amino acid sequence of a VH CDR1, VH CDR2, and VH CDR3 of the CD20 binding domain of glofitamab, and a VL CDR1, VL CDR2, and VL CDR3 of the CD20 binding domain of glofitamab. In one embodiment, the CD3×CD20 antibody comprises an amino acid sequence of a VH CDR1, VH CDR2, and VH CDR3 of the CD3 binding domain of glofitamab, a VL CDR1, VL CDR2, and VL CDR3 of the CD3 binding domain of glofitamab, a VH CDR1, VH CDR2, and VH CDR3 of the CD20 binding domain of glofitamab, and a VL CDR1, VL CDR2, and VL CDR3 of the CD20 binding domain of glofitamab. In one embodiment, the CD3×CD20 antibody comprises an amino acid sequence of a VH of the CD3 binding domain of glofitamab. In one embodiment, the CD3×CD20 antibody comprises an amino acid sequence of a VL of the CD3 binding domain of glofitamab. In one embodiment, the CD3×CD20 antibody comprises an amino acid sequence of a VH of the CD20 binding domain of glofitamab. In one embodiment, the CD3×CD20 antibody comprises an amino acid sequence of a VL of the CD20 binding domain of glofitamab. In one embodiment, the CD3×CD20 antibody comprises an amino acid sequence of a VH of the CD3 binding domain of glofitamab, and a VL of the CD3 binding domain of glofitamab. In one embodiment, the CD3×CD20 antibody comprises an amino acid sequence of a VH of the CD20 binding domain of glofitamab, and a VL of the CD20 binding domain of glofitamab. In one embodiment, the CD3×CD20 antibody comprises an amino acid sequence of a VH of the CD3 binding domain of glofitamab, a VL of the CD3 binding domain of glofitamab, a VH of the CD20 binding domain of glofitamab, and a VL of the CD20 binding domain of glofitamab.

In one embodiment, the CD19 antibody comprises a heavy chain variable (VH) domain comprising a VH CDR 1, a VH CDR2, and a VH CDR3 having an amino acid sequence of SEQ ID NO:1, SEQ ID NO:2, and SEQ ID NO:3, respectively.

In one embodiment, the CD19 antibody comprises a light chain variable (VL) domain comprising a VL CDR1, a VL CDR2, and a VL CDR3 having an amino acid sequence of SEQ ID NO:4, SEQ ID NO:5, and SEQ ID NO:6, respectively.

In one embodiment, the CD19 antibody comprises a heavy chain variable (VH) domain comprising a VH CDR 1, a VH CDR2, and a VH CDR3 having an amino acid sequence of SEQ ID NO:1, SEQ ID NO:2, and SEQ ID NO:3, respectively, and a light chain variable (VL) domain comprising a VL CDR1, a VL CDR2, and a VL CDR3 having an amino acid sequence of SEQ ID NO:4, SEQ ID NO:5, and SEQ ID NO:6, respectively.

In one embodiment, the CD19 antibody comprises a VH domain having an amino acid sequence that is about 90%, 95%, 98%, 99%, or 100% identical to the amino acid sequence of SEQ ID NO:19. In one embodiment, the CD19 antibody comprises a VH domain having an amino acid sequence that is about 90% identical to the amino acid sequence of SEQ ID NO:19. In another embodiment, the CD19 antibody comprises a VH domain having an amino acid sequence that is about 95% identical to the amino acid sequence of SEQ ID NO:19. In another embodiment, the CD19 antibody comprises a VH domain having an amino acid sequence that is about 98% identical to the amino acid sequence of SEQ ID NO: 19. In another embodiment, the CD19 antibody comprises a VH domain having an amino acid sequence that is about 99% identical to the amino acid sequence of SEQ ID NO:19. In another embodiment, the CD19 antibody comprises a VH domain having an amino acid sequence that is about 100% identical to the amino acid sequence of SEQ ID NO:19.

In one embodiment, the CD19 antibody comprises a VL domain having an amino acid sequence that is about 90%, 95%, 98%, 99%, or 100% identical to the amino acid sequence of SEQ ID NO:20. In one embodiment, the CD19 antibody comprises a VL domain having an amino acid sequence that is about 90% identical to the amino acid sequence of SEQ ID NO:20. In another embodiment, the CD19 antibody comprises a VL domain having an amino acid sequence that is about 95% identical to the amino acid sequence of SEQ ID NO:20. In another embodiment, the CD19 antibody comprises a VL domain having an amino acid sequence that is about 98% identical to the amino acid sequence of SEQ ID NO:20. In another embodiment, the CD19 antibody comprises a VL domain having an amino acid sequence that is about 99% identical to the amino acid sequence of SEQ ID NO:20. In another embodiment, the CD19 antibody comprises a VL domain having an amino acid sequence that is about 100% identical to the amino acid sequence of SEQ ID NO:20.

In one embodiment, the CD19 antibody comprises a VH domain having an amino acid sequence that is about 90%, 95%, 98%, 99%, or 100% identical to the amino acid sequence of SEQ ID NO:19, and a VL domain having an amino acid sequence that is about 90%, 95%, 98%, 99%, or 100% identical to the amino acid sequence of SEQ ID NO:20. In one embodiment, the CD19 antibody comprises a VH domain having an amino acid sequence that is about 90% identical to the amino acid sequence of SEQ ID NO: 19, and a VL domain having an amino acid sequence that is about 90% identical to the amino acid sequence of SEQ ID NO:20. In another embodiment, the CD19 antibody comprises a VH domain having an amino acid sequence that is about 90% identical to the amino acid sequence of SEQ ID NO:19, and a VL domain having an amino acid sequence that is about 90% identical to the amino acid sequence of SEQ ID NO:20. In another embodiment, the CD19 antibody comprises a VH domain having an amino acid sequence that is about 95% identical to the amino acid sequence of SEQ ID NO:19, and a VL domain having an amino acid sequence that is about 95% identical to the amino acid sequence of SEQ ID NO:20. In another embodiment, the CD19 antibody comprises a VH domain having an amino acid sequence that is about 98% identical to the amino acid sequence of SEQ ID NO:19, and a VL domain having an amino acid sequence that is about 98% identical to the amino acid sequence of SEQ ID NO:20. In another embodiment, the CD19 antibody comprises a VH domain having an amino acid sequence that is about 99% identical to the amino acid sequence of SEQ ID NO:19, and a VL domain having an amino acid sequence that is about 99% identical to the amino acid sequence of SEQ ID NO:20. In another embodiment, the CD19 antibody comprises a VH domain having an amino acid sequence that is about 100% identical to the amino acid sequence of SEQ ID NO:19, and a VL domain having an amino acid sequence that is about 100% identical to the amino acid sequence of SEQ ID NO:20.

In a specific embodiment, the CD19 antibody is tafasitamab. In one embodiment, the CD19 antibody is a biosimilar of tafasitamab. In one embodiment, the CD19 antibody is a bioequivalent of tafasitamab. In one embodiment, the CD19 antibody comprises an amino acid sequence of a VH CDR1, VH CDR2, and VH CDR3 of tafasitamab. In one embodiment, the CD19 antibody comprises an amino acid sequence of a VL CDR1, VL CDR2, and VL CDR3 of tafasitamab. In one embodiment, the CD19 antibody comprises an amino acid sequence of a VH CDR1, VH CDR2, and VH CDR3 of tafasitamab, and an amino acid sequence of a VL CDR1, VL CDR2, and VL CDR3 of tafasitamab. In one embodiment, the CD19 antibody comprises an amino acid sequence of a VH of tafasitamab. In one embodiment, the CD19 antibody comprises an amino acid sequence of a VL of tafasitamab. In one embodiment, the CD19 antibody comprises an amino acid sequence of a VH of tafasitamab, and an amino acid sequence of a VL of tafasitamab.

### Compounds

In some aspects, provided are methods of treating lymphoma using a combination treatment comprising a compound having the structure

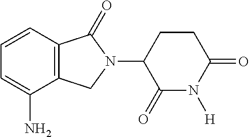 [SMILES: Nc1cccc2c1CN(C1CCC(=O)NC1=O)C2=O]

(i.e., 3-(4-amino-1-oxo 1,3-dihydro-2H-isoindol-2-yl) piperidine-2,6-dione (Compound A)). In some embodiments, the compound is a pharmaceutically acceptable salt of Compound A. In some embodiments, the compound is a solvate of Compound A. In some embodiments, the compound is a stereoisomer of Compound A. Other compounds useful in the methods provided herein are compounds (e.g., Compound A) that are racemic, stereomerically enriched or stereomerically pure, and pharmaceutically acceptable salts, solvates, hydrates, stereoisomers, clathrates, and prodrugs thereof.

The compounds useful in the methods provided herein can either be commercially purchased or prepared according to the methods described in the patents or patent publications disclosed herein. For example, the compounds can be obtained via standard, synthetic methods (see e.g., U.S. Pat. No. 5,635,517, incorporated herein by reference in its entirety). Further, optically pure compounds can be asymmetrically synthesized or resolved using known resolving agents or chiral columns as well as other standard synthetic organic chemistry techniques. As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of Compound A that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of Compound A that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, .alpha.-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

Compounds provided herein (e.g., Compound A) can comprises one or more chiral centers, and can exist as racemic mixtures of enantiomers or mixtures of diastereomers. The methods provided herein encompass the use of stereomerically pure forms of such compounds, as well as the use of mixtures of those forms. For example, mixtures comprising equal or unequal amounts of the enantiomers of a particular compound (e.g., Compound A) may be used in methods provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means a composition that comprises one stereoisomer of a compound (e.g., Compound A) and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4.4 Methods of Treatment

The methods provided herein are useful for the treatment of lymphoma, including, but not limited to Non-Hodgkin lymphomas such as Diffuse Large B-Cell Lymphoma (DLBCL).

Non-Hodgkin lymphomas (NHL) are a diverse group of malignancies that are predominately of B-cell origin. NHL may develop in any organs associated with lymphatic system such as spleen, lymph nodes or tonsils and can occur at any age. NHL is often marked by enlarged lymph nodes, fever, and weight loss. NHL is classified as either B-cell or T-cell NHL. Lymphomas related to lymphoproliferative disorders following bone marrow or stem cell transplantation are usually B-cell NHL. NHL has been divided into low-, intermediate-, and high-grade categories by virtue of their natural histories (see “The Non-Hodgkin's Lymphoma Pathologic Classification Project,” Cancer 49 (1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

DLBCL is the most common subtype NHL. Since the introduction of the anti-CD20 antibody rituximab, approximately 50% to 70% of patients may achieve cure with initial standard-of-care immunochemotherapy. However, for patients who are refractory to or relapse after frontline therapy, prognosis is poor. Salvage chemotherapy followed by high-dose chemotherapy and autologous stem-cell transplantation (ASCT) has some benefit in this setting and is associated with significant toxicities. Moreover, most patients are ineligible for this approach and have fewer treatment options. Thus, a significant need remains to provide effective treatment options for patients with relapsed or refractory (R/R) DLBCL who are ineligible for ASCT (Coiffier B et al. Hematology Am Soc Hematol Educ Program. 2016; (1):366-78).

In an exemplary embodiment, provided herein are methods for treating lymphoma in a subject comprising administering to the subject a combination of a CD19 antibody, a CD3×CD20 antibody, and Compound A.

In one aspect, provided herein is a method of treating lymphoma in a subject comprising administering to the subject: (a) an antibody that binds CD19 (CD19 antibody); (b) a multispecific antibody, wherein the multispecific antibody comprises a first binding domain that binds to CD3 and a second binding domain that binds to CD20 (CD3×CD20 antibody); and (c) a compound having the structure:

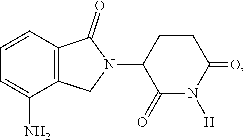 [SMILES: Nc1cccc2c1CN(C1CCC(=O)NC1=O)C2=O]

(Compound A). In some embodiments, the compound is a pharmaceutically acceptable salt, solvate, or stereoisomer thereof of Compound A.

In specific embodiments, the subject is administered a first dose of the CD19 antibody at least one day prior to administration to the subject of a first dose of the Compound A or pharmaceutically acceptable salt, solvate or stereoisomer thereof. In other embodiments, the subject is administered a first dose of the CD19 antibody at least one day prior to administration to the subject of a first dose of the CD3×CD20 antibody.

In yet other embodiments, the subject is administered a first dose of the CD19 antibody at least one day prior to administration to the subject of a first dose of the Compound A or pharmaceutically acceptable salt, solvate or stereoisomer thereof, and the subject is administered a first dose of the CD19 antibody at least one day prior to administration to the subject of a first dose of the CD3×CD20 antibody.

In certain embodiments, the subject is a subject in need thereof.

In one embodiment, the lymphoma is Non-Hodgkin lymphoma. In another embodiment, the Non-Hodgkin lymphoma is DLBCL. In another embodiment, the DLBCL is relapsed, refractory, or relapsed and refractory DLBCL. In one embodiment, the DLBCL is relapsed DLBCL. In one embodiment, the DLBCL is refractory DLBCL. In one embodiment, the DLBCL is relapsed and refractory DLBCL. In another embodiment, the DLBCL is primary refractory DLBCL. In another embodiment, the DLBCL is first line DLBCL.

In one embodiment, the lymphoma is a CD20-expressing lymphoma. In another embodiment, the lymphoma is a CD19-expressing lymphoma. In another embodiment, the lymphoma is a CD20-expressing and CD19-expressing lymphoma.

In one embodiment, the subject has received a prior CAR-T therapy.

In one embodiment, the subject has not received stem cell transplantation. In another embodiment, the subject is not eligible for stem cell transplantation. In another embodiment, the stem cell transplantation is autologous stem cell transplantation.

In one embodiment, the subject received treatment for lymphoma prior to the method. In an embodiment, the treatment comprises chemoimmunotherapy, an anti-CD20 antibody, or a combination thereof.

In some embodiments, provided is a method of treating a lymphoma in a subject, comprising administering to the subject (i) a first polypeptide comprising a first means capable of binding to CD3 and a second means capable of binding to CD20, (ii) a second polypeptide comprising a third means capable of binding to CD19, and (iii) a compound. In some embodiments, provided is a method of treating DLBCL in a subject, comprising administering to the subject (i) a first polypeptide comprising a first means capable of binding to CD3 and a second means capable of binding to CD20, (ii) a second polypeptide comprising a third means capable of binding to CD19, and (iii) a compound. In some embodiments, provided is a method of inhibiting the growth or proliferation of lymphoma cells in a subject, comprising administering to the subject (i) a first polypeptide comprising a first means capable of binding to CD3 and a second means capable of binding to CD20, (ii) a second polypeptide comprising a third means capable of binding to CD19, and (iii) a compound. In some embodiments, provided is a method of inhibiting the growth or proliferation of DLBCL cells in a subject, comprising administering to the subject (i) a first polypeptide comprising a first means capable of binding to CD3 and a second means capable of binding to CD20, (ii) a second polypeptide comprising a third means capable of binding to CD19, and (iii) a compound. In a specific embodiment, the compound is Compound A. Other compounds useful in the methods provided herein are compounds that are racemic, stereomerically enriched or stereomerically pure, and pharmaceutically acceptable salts, solvates, hydrates, stereoisomers, clathrates, and prodrugs thereof of Compound A. In some embodiments, the first polypeptide is a multispecific antibody. In some embodiments, the first polypeptide is a bispecific antibody. In one embodiment, the multispecific antibody comprises a bispecific antibody. In one embodiment, the first means is a CD3 binding domain. In one embodiment, the second means is a CD20 binding domain. In one embodiment, the first means is a CD3 antigen binding fragment. In one embodiment, the second means is a CD20 antigen binding fragment. In a specific embodiment, the bispecific antibody is a CD3×CD20 antibody. In one embodiment, the third means is a CD19 binding domain. In one embodiment, the third means is a CD19 antigen binding fragment. In some embodiments, the second polypeptide is a CD19 antibody. In certain embodiments, the subject is a subject in need thereof. In a specific embodiment, the subject is a human subject.

### Dosage Regimens

In one aspect, the CD19 antibody, CD3×CD20 antibody, and the Compound A provided are administered according to a dosage regimen provided herein. However, the CD19 antibody, CD3×CD20 antibody, and Compound A can be administered by any method known in the art. One skilled in the art would appreciate that the route and/or mode of administration may vary depending upon the desired results.

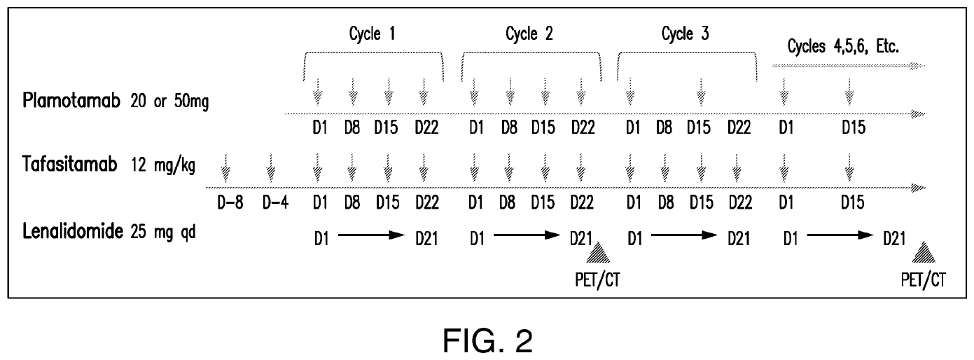

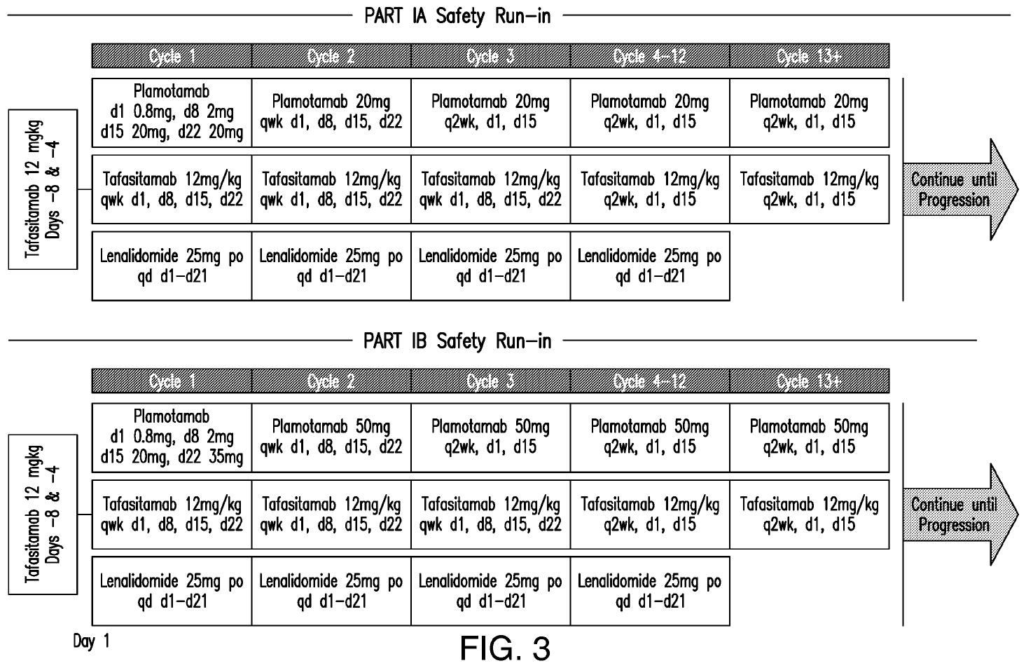

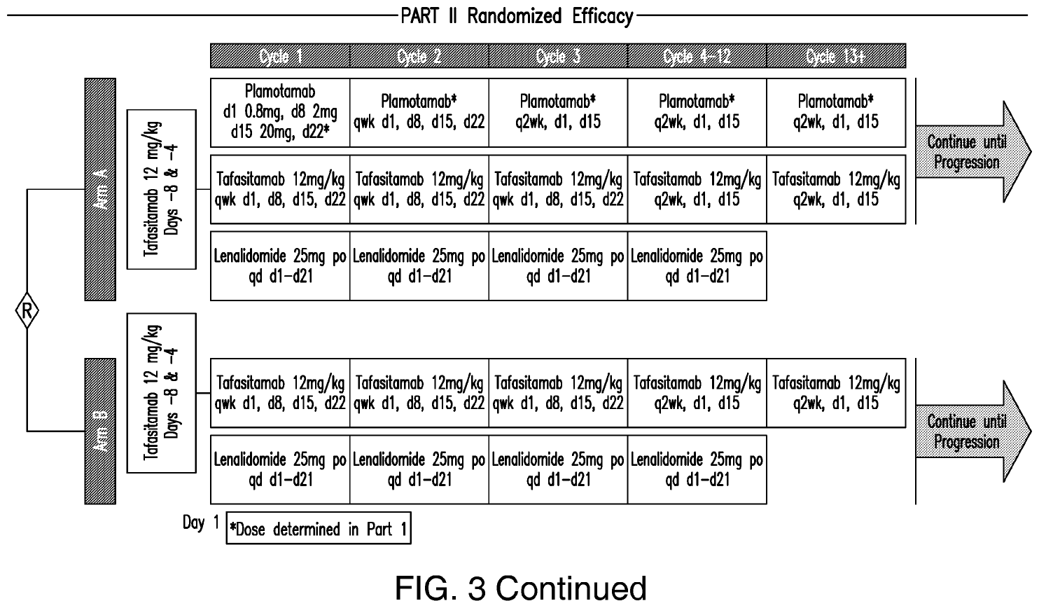

In one embodiment, provided herein in a method comprising a dosing regimen provided in FIG. 2. In another embodiment, provided herein in a method comprising a dosing regimen provided in FIG. 3. In yet another embodiment, provided herein in a method comprising a dosing regimen provided in Example 1.

In one embodiment, the method comprises cyclic administration of the CD19 antibody. In one embodiment, the method comprises cyclic administration of Compound A or a pharmaceutically acceptable salt, solvate, or stereoisomer thereof. In one embodiment, the method comprises cyclic administration of the CD3×CD20 antibody. In one embodiment, the method comprises cyclic administration of the CD19 antibody, Compound A or a pharmaceutically acceptable salt, solvate, or stereoisomer thereof, and the CD3×CD20 antibody.

In one embodiment, each cycle of the cyclic administration is 28 days. In another embodiment, the cyclic administration comprises about one cycle, two cycles, three cycles, four cycles, five cycles, six cycles, seven cycles, eight cycles, nine cycles, ten cycles, eleven cycles, twelve cycles, or more than twelve cycles. In another embodiment, the cyclic administration comprises about one cycle. In another embodiment, the cyclic administration comprises about two cycles. In another embodiment, the cyclic administration comprises about three cycles. In another embodiment, the cyclic administration comprises about four cycles. In another embodiment, the cyclic administration comprises about five cycles. In another embodiment, the cyclic administration comprises about six cycles. In another embodiment, the cyclic administration comprises about seven cycles. In another embodiment, the cyclic administration comprises about eight cycles. In another embodiment, the cyclic administration comprises about nine cycles. In another embodiment, the cyclic administration comprises about ten cycles. In another embodiment, the cyclic administration comprises about eleven cycles. In another embodiment, the cyclic administration comprises about twelve cycles. In another embodiment, the cyclic administration comprises more than twelve cycles.

In one embodiment, the first dose of the CD19 antibody is administered to the subject prior to day 1 of the first cycle of the cyclic administration. In one embodiment, the first dose of the CD19 antibody is administered to the subject at least one day, two days, three days, four days, five days, six days, seven days, eight days, nine days, ten days, or more than ten days prior to day 1 of the first cycle of the cyclic administration. In one embodiment, the first dose of the CD19 antibody is administered to the subject at least one day prior to day 1 of the first cycle of the cyclic administration. In another embodiment, the first dose of the CD19 antibody is administered to the subject at least two days prior to day 1 of the first cycle of the cyclic administration. In another embodiment, the first dose of the CD19 antibody is administered to the subject at least three days prior to day 1 of the first cycle of the cyclic administration. In another embodiment, the first dose of the CD19 antibody is administered to the subject at least four days prior to day 1 of the first cycle of the cyclic administration. In another embodiment, the first dose of the CD19 antibody is administered to the subject at least five days prior to day 1 of the first cycle of the cyclic administration. In another embodiment, the first dose of the CD19 antibody is administered to the subject at least six days prior to day 1 of the first cycle of the cyclic administration. In another embodiment, the first dose of the CD19 antibody is administered to the subject at least seven days prior to day 1 of the first cycle of the cyclic administration. In another embodiment, the first dose of the CD19 antibody is administered to the subject at least eight days prior to day 1 of the first cycle of the cyclic administration. In another embodiment, the first dose of the CD19 antibody is administered to the subject at least nine days prior to day 1 of the first cycle of the cyclic administration. In another embodiment, the first dose of the CD19 antibody is administered to the subject at least ten days prior to day 1 of the first cycle of the cyclic administration. In another embodiment, the first dose of the CD19 antibody is administered to the subject more than ten days prior to day 1 of the first cycle of the cyclic administration. In another embodiment, the first dose of the CD19 antibody is administered to the subject four days prior to day 1 of the first cycle of the cyclic administration. In another embodiment, the first dose of the CD19 antibody is administered to the subject eight days prior to day 1 of the first cycle of the cyclic administration. In another embodiment, the CD19 antibody is administered to the subject four days and eight days prior to day 1 of the first cycle of the cyclic administration.

In one embodiment, the CD19 antibody is administered to the subject on day(s) 1, 8, 15, and/or 22 of a cycle of the cyclic administration. In one embodiment, the CD19 antibody is administered to the subject on day(s) 1, 8, 15, and 22 of a cycle of the cyclic administration. In one embodiment, the CD19 antibody is administered to the subject on day(s) 1, 8, 15, or 22 of a cycle of the cyclic administration. In another embodiment, the CD19 antibody is administered to the subject on day 1 of a cycle of the cyclic administration. In another embodiment, the CD19 antibody is administered to the subject on day 8 of a cycle of the cyclic administration. In another embodiment, the CD19 antibody is administered to the subject on day 15 of a cycle of the cyclic administration. In another embodiment, the CD19 antibody is administered to the subject on day 22 of a cycle of the cyclic administration. In another embodiment, the CD19 antibody is administered to the subject on days 1 and 15 of a cycle of the cyclic administration. In another embodiment, the CD19 antibody is administered to the subject on days 1, 8, 15, and 22 of each of cycles 1-3 of the cyclic administration. In another embodiment, the CD19 antibody is administered to the subject on days 1 and 15 for cycle 4 and onwards of the cyclic administration. In another embodiment, the CD19 antibody is administered to the subject on days 1 and 15 for cycles 4-6 of the cyclic administration. In another embodiment, the CD19 antibody is administered to the subject every 6 to 8 days in a cycle of the cyclic administration.

In one embodiment, Compound A or pharmaceutically acceptable salt, solvate, or stereoisomer thereof is administered to the subject for 21 continuous days of the cyclic administration. In another embodiment, Compound A or pharmaceutically acceptable salt, solvate, or stereoisomer thereof is administered to the subject from day 1 to day 21 of the cyclic administration. In another embodiment, Compound A or pharmaceutically acceptable salt, solvate, or stereoisomer thereof is administered to the subject for 21 days followed by seven days of rest in a 28 day cycle of the cyclic administration.

In one embodiment, the CD3×CD20 antibody is administered to the subject on day(s) 1, 8, 15, and/or 22 of a cycle of the cyclic administration. In another embodiment, the CD3×CD20 antibody is administered to the subject on day(s) 1, 8, 15, and 22 of a cycle of the cyclic administration. In another embodiment, the CD3×CD20 antibody is administered to the subject on day(s) 1, 8, 15, or 22 of a cycle of the cyclic administration. In another embodiment, the CD3×CD20 antibody is administered to the subject on day 1 of a cycle of the cyclic administration. In another embodiment, the CD3×CD20 antibody is administered to the subject on day 8 of a cycle of the cyclic administration. In another embodiment, the CD3×CD20 is administered to the subject on day 15 of a cycle of the cyclic administration. In another embodiment, the CD3×CD20 antibody is administered to the subject on day 22 of a cycle of the cyclic administration. In another embodiment, the CD3×CD20 antibody is administered to the subject on days 1 and 15 of a cycle of the cyclic administration. In another embodiment, the CD3×CD20 antibody is administered to the subject on days 1, 8, 15, and 22 of cycle 1 and cycle 2 of the cyclic administration. In another embodiment, the CD3×CD20 antibody is administered to the subject on days 1 and 15 for cycle 3 and onwards of the cyclic administration. In another embodiment, the CD3×CD20 antibody is administered to the subject on days 1 and 15 for cycles 3-6 of the cyclic administration. In another embodiment, the CD3×CD20 antibody is administered to the subject every 6 to 8 days in a cycle of the cyclic administration.

In one embodiment, the CD19 antibody is administered to the subject in an amount of about 1 mg/kg to about 20 mg/kg per day. In one embodiment, the CD19 antibody is administered to the subject in an amount of about 1 mg/kg per day. In another embodiment, the CD19 antibody is administered to the subject in an amount of about 2 mg/kg per day. In another embodiment, the CD19 antibody is administered to the subject in an amount of about 3 mg/kg per day. In another embodiment, the CD19 antibody is administered to the subject in an amount of about 4 mg/kg per day. In another embodiment, the CD19 antibody is administered to the subject in an amount of about 5 mg/kg per day. In another embodiment, the CD19 antibody is administered to the subject in an amount of about 6 mg/kg per day. In another embodiment, the CD19 antibody is administered to the subject in an amount of about 7 mg/kg per day. In another embodiment, the CD19 antibody is administered to the subject in an amount of about 8 mg/kg per day. In another embodiment, the CD19 antibody is administered to the subject in an amount of about 9 mg/kg per day. In another embodiment, the CD19 antibody is administered to the subject in an amount of about 10 mg/kg per day. In another embodiment, the CD19 antibody is administered to the subject in an amount of about 11 mg/kg per day. In another embodiment, the CD19 antibody is administered to the subject in an amount of about 12 mg/kg per day. In another embodiment, the CD19 antibody is administered to the subject in an amount of about 13 mg/kg per day. In another embodiment, the CD19 antibody is administered to the subject in an amount of about 14 mg/kg per day. In another embodiment, the CD19 antibody is administered to the subject in an amount of about 15 mg/kg per day. In another embodiment, the CD19 antibody is administered to the subject in an amount of about 16 mg/kg per day. In another embodiment, the CD19 antibody is administered to the subject in an amount of about 17 mg/kg per day. In another embodiment, the CD19 antibody is administered to the subject in an amount of about 18 mg/kg per day. In another embodiment, the CD19 antibody is administered to the subject in an amount of about 19 mg/kg per day. In another embodiment, the CD19 antibody is administered to the subject in an amount of about 20 mg/kg per day.

In one embodiment, the Compound A or pharmaceutically acceptable salt, solvate, or stereoisomer thereof is administered to the subject in an amount of about 1 mg to about 30 mg per day. In another embodiment, the Compound A or pharmaceutically acceptable salt, solvate, or stereoisomer thereof is administered to the subject in an amount of about 2.5 mg, 5 mg, 10 mg, 15 mg, 20 mg, or 25 mg per day. In another embodiment, the Compound A or pharmaceutically acceptable salt, solvate, or stereoisomer thereof is administered to the subject in an amount of about 2.5 mg per day. In another embodiment, the Compound A or pharmaceutically acceptable salt, solvate, or stereoisomer thereof is administered to the subject in an amount of about 5 mg per day. In another embodiment, the Compound A or pharmaceutically acceptable salt, solvate, or stereoisomer thereof is administered to the subject in an amount of about 10 mg per day. In another embodiment, the Compound A or pharmaceutically acceptable salt, solvate, or stereoisomer thereof is administered to the subject in an amount of about 15 mg per day. In another embodiment, the Compound A or pharmaceutically acceptable salt, solvate, or stereoisomer thereof is administered to the subject in an amount of about 20 mg per day. In another embodiment, the Compound A or pharmaceutically acceptable salt, solvate, or stereoisomer thereof is administered to the subject in an amount of about 25 mg per day.

In one embodiment, the CD3×CD20 antibody is administered to the subject in an amount of about 0.8 mg to about 100 mg per day. In one embodiment, the CD3×CD20 antibody is administered to the subject in an amount of about 0.8 mg to about 50 mg per day. In one embodiment, the CD3×CD20 antibody is administered to the subject in an amount of about 0.8 mg to about 20 mg per day. In another embodiment, the CD3×CD20 antibody is administered to the subject in an amount of about 0.8 mg per day. In another embodiment, the CD3×CD20 antibody is administered to the subject in an amount of about 1 mg per day. In another embodiment, the CD3×CD20 antibody is administered to the subject in an amount of about 2 mg per day. In another embodiment, the CD3×CD20 antibody is administered to the subject in an amount of about 3 mg per day. In another embodiment, the CD3×CD20 antibody is administered to the subject in an amount of about 4 mg per day. In another embodiment, the CD3×CD20 antibody is administered to the subject in an amount of about 5 mg per day. In another embodiment, the CD3×CD20 antibody is administered to the subject in an amount of about 10 mg per day. In another embodiment, the CD3×CD20 antibody is administered to the subject in an amount of about 15 mg per day. In another embodiment, the CD3×CD20 antibody is administered to the subject in an amount of about 20 mg per day. In another embodiment, the CD3×CD20 antibody is administered to the subject in an amount of about 25 mg per day. In another embodiment, the CD3×CD20 antibody is administered to the subject in an amount of about 30 mg per day. In another embodiment, the CD3×CD20 antibody is administered to the subject in an amount of about 35 mg per day. In another embodiment, the CD3×CD20 antibody is administered to the subject in an amount of about 40 mg per day. In another embodiment, the CD3×CD20 antibody is administered to the subject in an amount of about 5 mg per day. In another embodiment, the CD3×CD20 antibody is administered to the subject in an amount of about 50 mg per day. In another embodiment, the CD3×CD20 antibody is administered to the subject in an amount of about 55 mg per day. In another embodiment, the CD3×CD20 antibody is administered to the subject in an amount of about 60 mg per day. In another embodiment, the CD3×CD20 antibody is administered to the subject in an amount of about 65 mg per day. In another embodiment, the CD3×CD20 antibody is administered to the subject in an amount of about 70 mg per day. In another embodiment, the CD3×CD20 antibody is administered to the subject in an amount of about 75 mg per day. In another embodiment, the CD3×CD20 antibody is administered to the subject in an amount of about 80 mg per day. In another embodiment, the CD3×CD20 antibody is administered to the subject in an amount of about 85 mg per day. In another embodiment, the CD3×CD20 antibody is administered to the subject in an amount of about 90 mg per day. In another embodiment, the CD3×CD20 antibody is administered to the subject in an amount of about 95 mg per day. In another embodiment, the CD3×CD20 antibody is administered to the subject in an amount of about 100 mg per day.

In one embodiment, the first dose of the CD3×CD20 antibody is on day 1 of the first cycle of the cyclic administration.

In one embodiment, the CD3×CD20 antibody is administered to the subject in an amount of about 0.8 mg on day 1 of the first cycle, about 2 mg on day 8 of the first cycle, about 20 mg on days 15 and 22 of the first cycle, and about 20 mg per day for any subsequent cycles. In another embodiment, the CD3×CD20 antibody is administered to the subject in an amount of about 0.8 mg on day 1 of the first cycle, about 2 mg on day 8 of the first cycle, about 20 mg on day 15 of the first cycle, about 35 mg on day 22 of the first cycle, and about 50 mg per day for any subsequent cycles.

In one embodiment, the first dose of the Compound A or pharmaceutically acceptable salt, solvate, or stereoisomer thereof is on day 1 of the first cycle of the cyclic administration.

In one embodiment, the first dose of the CD3×CD20 antibody and the first dose of the Compound A or pharmaceutically acceptable salt, solvate, or stereoisomer thereof are both on day 1 of the first cycle of the cyclic administration.

In one embodiment, the CD19 antibody is administered to the subject once a week in a cycle of the cyclic administration. In another embodiment, the CD19 antibody is administered to the subject once a week for cycles 1-3 of the cyclic administration. In another embodiment, the CD19 antibody is administered to the subject every two weeks in a cycle of the cyclic administration. In another embodiment, the CD19 antibody is administered to the subject every two weeks for cycles 4 and onwards of the cyclic administration.

In one embodiment, the CD3×CD20 antibody is administered to the subject once a week in a cycle of the cyclic administration. In another embodiment, the CD3×CD20 antibody is administered to the subject once a week for cycles 1 and 2 of the cyclic administration. In another embodiment, the CD3×CD20 antibody is administered to the subject every two weeks in a cycle of the cyclic administration. In another embodiment, the CD3×CD20 antibody is administered to the subject every two weeks for cycles 3 and onwards of the cyclic administration.

In one embodiment, the CD3×CD20 antibody and the CD19 antibody are each administered to the subject in at most 4 days in a cycle of the cyclic administration. In another embodiment, the CD3×CD20 antibody is administered to the subject one day in a cycle of the cyclic administration. In another embodiment, the CD3×CD20 antibody is administered to the subject two days in a cycle of the cyclic administration. In another embodiment, the CD3×CD20 antibody is administered to the subject three days in a cycle of the cyclic administration. In another embodiment, the CD3×CD20 antibody is administered to the subject four days in a cycle of the cyclic administration. In another embodiment, the CD19 antibody is administered to the subject one day in a cycle of the cyclic administration. In another embodiment, the CD19 antibody is administered to the subject two days in a cycle of the cyclic administration. In another embodiment, the CD19 antibody is administered to the subject three days in a cycle of the cyclic administration. In another embodiment, the CD19 antibody is administered to the subject four days in a cycle of the cyclic administration. In one embodiment, the CD3×CD20 antibody and the CD19 antibody are each administered to the subject one day in a cycle of the cyclic administration. In one embodiment, the CD3×CD20 antibody and the CD19 antibody are each administered to the subject two days in a cycle of the cyclic administration. In one embodiment, the CD3×CD20 antibody and the CD19 antibody are each administered to the subject three days in a cycle of the cyclic administration. In one embodiment, the CD3×CD20 antibody and the CD19 antibody are each administered to the subject four days in a cycle of the cyclic administration.

In one embodiment, the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 20 mg, wherein about 20 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 35 mg, wherein about 50 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 20 mg, wherein about 20 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment, and wherein each cycle of the cyclic administration is 28 days.

In one embodiment, the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 35 mg, wherein about 50 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment, and wherein each cycle of the cyclic administration is 28 days.

In one embodiment, the method comprises cyclic administration of the CD19 antibody to the subject, wherein from about 10 mg/kg to about 15 mg/kg of the CD19 antibody is administered to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during a course of treatment, wherein from about 10 mg/kg to about 15 mg/kg of the CD19 antibody is administered to the subject 8 days and 4 days prior to day 1 of a first cycle of the cyclic administration, and wherein each cycle of the cyclic administration is 28 days.

In one embodiment, the method comprises cyclic administration of the CD19 antibody to the subject, wherein about 12 mg/kg of the CD19 antibody is administered to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during a course of treatment, wherein about 12 mg/kg of the CD19 antibody is administered to the subject 8 days and 4 days prior to day 1 of a first cycle of the cyclic administration, and wherein each cycle of the cyclic administration is 28 days.

In one embodiment, the method comprises cyclic administration of the Compound A or pharmaceutically acceptable salt, solvate, or stereoisomer thereof to the subject, wherein about 25 mg of the Compound A or pharmaceutically acceptable salt, solvate, or stereoisomer thereof is administered to the subject on day 1 and on every day for 21 days followed by 7 days of rest in each cycle of the cyclic administration, and wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering from about 0.6 to about 1 mg of the CD3×CD20 antibody to the subject on day 1, from about 1.8 mg to about 2.2 mg on about day 8, and from about 18 mg to about 22 mg on about day 15 and 22 of the first cycle; administering about 20 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment;
(b) administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration; and
wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering from about 0.6 to about 1 mg of the CD3×CD20 antibody to the subject on day 1, from about 1.8 mg to about 2.2 mg on about day 8, from about 18 mg to about 22 mg on about day 15, and from about 33 mg to about 36 mg on about day 22 of the first cycle; administering about 50 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment;
(b) administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration; and wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering about 0.8 mg of the CD3×CD20 antibody to the subject on day 1, about 2 mg on about day 8, and about 20 mg on day 15 and 22 of the first cycle; administering about 20 mg of the CD3×CD20 antibody to the subject on day 1 and every 7 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment;
(b) administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering about 0.8 mg of the CD3×CD20 antibody to the subject on day 1, about 2 mg on about day 8, about 20 mg on day 15, and about 35 mg on day 22 of the first cycle; administering about 50 mg of the CD3×CD20 antibody to the subject on day 1 and every 7 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment;
(b) administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and
wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering about 0.8 mg of the CD3×CD20 antibody to the subject on day 1, about 2 mg on day 8, and about 20 mg on days 15 and 22 of the first cycle; administering about 20 mg of the CD3×CD20 antibody to the subject on days 1, 8, 15, and 22 of the second cycle of the cyclic administration, wherein about 20 mg of the CD3×CD20 antibody is administered to the subject on days 1 and 15 of cycle 3 and of any subsequent cycle during a course of treatment;
(b) administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and
wherein each cycle of the cyclic administration is 28 days.

In one embodiment, the method comprises cyclic administration of the CD19 antibody, the CD3×CD20 antibody, and the Compound A or pharmaceutically acceptable salt, solvate, or stereoisomer thereof to the subject, and wherein the method comprises:
(a) administering about 0.8 mg of the CD3×CD20 antibody to the subject on day 1, about 2 mg on day 8, about 20 mg on day 15, and about 35 mg on day 22 of the first cycle; administering about 50 mg of the CD3×CD20 antibody to the subject on days 1, 8, 15, and 22 of the second cycle of the cyclic administration, wherein about 50 mg of the CD3×CD20 antibody is administered to the subject on days 1 and 15 of cycle 3 and of any subsequent cycle during a course of treatment;
(b) administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and
(c) administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and
wherein each cycle of the cyclic administration is 28 days.

In one embodiment, the CD19 antibody is not administered to the subject on day 4 of the first cycle of the cyclic administration. In another embodiment, the CD3×CD20 antibody is not administered to the subject on day 4 of the first cycle of the cyclic administration.

In one embodiment, the method further comprises determining PET-CT after every two cycles of the cyclic administration.

In one embodiment, the CD19 antibody is tafasitamab. In one embodiment, the CD3×CD20 antibody is plamotamab. In one embodiment, the CD19 antibody is tafasitamab and the CD3×CD20 antibody is plamotamab.

### Administration

The pharmaceutical compositions provided herein may be in a variety of forms. These include, for example, liquid, semi-solid and solid dosage forms, such as liquid solutions (e.g., injectable and infusible solutions), dispersions or suspensions. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibodies provided herein are administered by intravenous infusion or injection.

Pharmaceutical compositions typically must be sterile and stable under the conditions of manufacture and storage. Sterile injectable solutions can be prepared by incorporating the bispecific antibody in the required amount in an appropriate solvent with one or a combination of ingredients enumerated herein, as required, followed by filtered sterilization. Generally, dispersions are prepared by incorporating the bispecific antibody into a sterile vehicle that contains a basic dispersion medium and the required other ingredients from those enumerated herein.

Pharmaceutical compositions provided herein can be administered by any method known in the art. As will be appreciated by the skilled artisan, the route and/or mode of administration will vary depending upon the desired results.

In one embodiment, the Compound A or pharmaceutically acceptable salt, solvate, or stereoisomer thereof is administered orally to the subject. In another embodiment, the Compound A or pharmaceutically acceptable salt, solvate, or stereoisomer thereof is administered in a capsule or tablet to the subject.

In an exemplary embodiment, the route/mode of administration is intravenous injection. In one embodiment, the CD19 antibody is administered intravenously.

### Additional Agents

In one aspect, provided is a method for treating lymphoma in a subject, comprising administering to the subject a combination of a CD19 antibody, a CD3×CD20 antibody, and Compound A in combination with at least one other agent.

Administered “in combination”, as used herein, means that three (or more) different agents are administered to the subject during the course of the subject's affliction with the disorder, e.g., the three or more agents are administered after the human subject has been diagnosed with the tumor and before the tumor has been treated. In some embodiments, the administration of one agent is still occurring when the administration of the second begins, so that there is overlap in terms of administration. This is sometimes referred to herein as “simultaneous” or “concurrent administration”. In other embodiments, the administration of one agent ends before the administration of the other agent begins. In some embodiments of either case, the treatment is more effective because of combined administration. In some embodiments, administration is such that the reduction in a symptom, or other parameter related to the tumor is greater than what would be observed with one agent administered in the absence of the other. The effect of the agents on the subject can be partially additive, wholly additive, or greater than additive. The administration can be such that an effect of the first treatment administration is still detectable when the second is administered.

The combination described herein and the at least one other agent can be administered simultaneously, in the same or in separate compositions, or sequentially. For sequential administration, the at least one other agent can be administered prior to or following administration of the combination described herein.

When administered in combination, the CD19 antibody, the CD3×CD20 antibody, the Compound A, and the at least one other agent, or all, can be administered in an amount or dose that is higher, lower or the same than the amount or dosage of each therapeutic agent used individually, e.g., as a monotherapy. In some embodiments, the administered amount or dosage of the CD19 antibody, the CD3×CD20 antibody, the Compound A, and the at least one other agent, or all, is lower (e.g., at least 20%, at least 30%, at least 40%, or at least 50%) than the amount or dosage of each therapeutic agent used individually, e.g., as a monotherapy. In other embodiments, the amount or dosage of the CD19 antibody, the CD3×CD20 antibody, the Compound A, and the at least one other agent, or all, that results in a desired effect (e.g., treatment of tumor) is lower (e.g., at least 20%, at least 30%, at least 40%, or at least 50% lower) than the amount or dosage of each agent used individually, e.g., as a monotherapy, required to achieve the same therapeutic effect.

In one embodiment, the other agent is a premedication. In another embodiment, the premedication is administered prior to administration of the CD3×CD20 antibody.

In one embodiment, the premedication is dexamethasone. In another embodiment, the dexamethasone is administered intravenously at a dose of 20 mg about one hour prior to administration of the CD3×CD20 antibody.

In one embodiment, the premedication is diphenhydramine. In another embodiment, the diphenhydramine is administered at a dose of 25 mg by mouth or intravenously about thirty to sixty minutes prior to administration of the CD3×CD20 antibody. In another embodiment, the premedication is acetaminophen. In another embodiment, the acetaminophen is administered at a dose of 650 mg by mouth or intravenously about thirty to sixty minutes prior to administration of the CD3×CD20 antibody.

In one aspect, the other agent is administered to treat a side effect.

In one embodiment, the side effect is cytokine release syndrome (“CRS”). Symptoms of CRS may include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output. CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dysmetria, altered gait, and seizures.

In another embodiment, the side effect is indigestion. In another embodiment, the side effect is nausea. In another embodiment, the side effect is vomiting. In another embodiment, the side effect is neurotoxicity. In another embodiment, the side effect is an allergic reaction, hypersensitivity, or an infusion-related reaction. In another embodiment, the side effect is hematologic toxicity. In another embodiment, the side effect is tumor lysis syndrome.

In an exemplary embodiment, the other agent is a steroid. In one embodiment, the steroid is a corticosteroid. In another embodiment, the corticosteroid is a glucocorticoid. In another embodiment, the corticosteroid is selected from the group consisting of betamethasone, dexamethasone, prednisone, prednisolone, methylprednisolone, and triamcinolone. In another embodiment, the corticosteroid is selected from the group consisting of hydrocortisone, cortisone, and ethamethasoneb. In another embodiment, the steroid is fludrocortisone. In another embodiment, the steroid is dexamethasone.

In an exemplary embodiment, the other agent is an antihistamine. In one embodiment, the antihistamine is an H₁ antagonist. In another embodiment, the H₁ antagonist is selected from the group consisting of acrivastine, azelastine, bilastine, bromodiphenhydramine, brompheniramine, buclizine, carbinoxamine, cetirizine (Zyrtec®), chlorodiphenhydramine, chlorphenamine, clemastine, cyclizine, cyproheptadine, dexbrompheniramine, dexchlorpheniramine, dimenhydrinate, dimetindene, diphenhydramine, doxylamine, ebastine, embramine, fexofenadine (Allegra®), hydroxyzine (Vistaril®), loratadine (Claritin®), meclizine, mirtazapine, olopatadine, orphenadrine, phenindamine, pheniramine, phenyltoloxamine, promethazine, quetiapine (Seroquel®), rupatadine (Alergoliber®), tripelennamine, and triprolidine.

In an exemplary embodiment, the antihistamine is acrivastine. In one embodiment, the antihistamine is cetirizine. In another embodiment, the antihistamine is diphenhydramine. In another embodiment, the antihistamine is Benadryl®.

In an exemplary embodiment, the antihistamine is an H₁ inverse agonist. In one embodiment, the H₁ inverse agonist is selected from the group consisting of acrivastine, cetirizine, levocetirizine, desloratadine, and pyrilamine.

In an exemplary embodiment, the antihistamine is an H₂ antihistamine. In one embodiment, the H₂ antihistamine is an H₂ antagonist. In another embodiment, the H₂ antihistamine is an H₂ inverse agonist. In another embodiment, the H₂ antihistamine is selected from the group consisting of cimetidine, famotidine, lafutidine, nizatidine, ranitidine, roxatidine, and tiotidine.

In an exemplary embodiment, the other agent is an antiallergy agent. In one embodiment, the other agent is selected from the group consisting of antihistamines, glucocorticoids, epinephrine (adrenaline), mast cell stabilizers, antileukotriene agents, anti-cholinergics, and decongestants. In another embodiment, the other agent is a decongestant. In another embodiment, the other agent is an adrenaline releasing agent. In another embodiment, the other agent is levomethamphetamine, phenylpropanolamine, propylhexedrine (Benzedrex®), or loratadine. In another embodiment, the other agent is an α-adrenergic receptor agonist. In another embodiment, the other agent is naphazoline, oxymetazoline, phenylephrine, synephrine, tetryzoline, tramazoline, or xylometazoline.

In an exemplary embodiment, the other agent is an antinausea agent. In one embodiment, the other agent is an antiemetic agent. In another embodiment, the other agent is a 5-HT₃ receptor antagonist. In another embodiment, the other agent is a dolasetron (Anzemet®), granisetron (Kytril®, Sancuso®), ondansetron (Zofran®), tropisetron (Setrovel®, Navoban®), palonosetron (Aloxi®), mirtazapine (Remeron®). In another embodiment, the other agent is a dopamine antagonist. In another embodiment, the other agent is a 5-HT₃ receptor antagonist. In another embodiment, the other agent is domperidone (Motilium®), olanzapine (Zyprexa®), droperidol, haloperidol, chlorpromazine, prochlorperazine, alizapride, prochlorperazine (Compazine®, Stemzine®, Buccastem®, Stemetil®, Phenotil®), metoclopramide (Reglan®). In another embodiment, the other agent is a NK1 receptor antagonist. In another embodiment, the other agent is aprepitant or fosaprepitant (Emend®), casopitant, rolapitant (Varubi®). In an exemplary embodiment, the other agent is an anticholinergic. In another embodiment, the other agent is scopolamine.

In an exemplary embodiment, the other agent is an analgesic agent. In one embodiment, the other agent is an antipyretic agent. In another embodiment, the other agent is a salicylate, or a derivative thereof. In another embodiment, the salicylate is selected from the group consisting of aspirin, diflunisal, salsalate, and salicylic acid, or a derivative thereof. In another embodiment, the salicylate is selected from the group consisting of choline salicylate, magnesium salicylate, and sodium salicylate. In another embodiment, the other agent is aspirin. In another embodiment, the other agent is acetaminophen, or a derivative thereof. In another embodiment, the other agent is an NSAID, or a derivative thereof. In another embodiment, the NSAID is a propionic acid derivative. In another embodiment, the NSAID is selected from the group consisting of ibuprofen, dexibuprofen, naproxen, fenoprofen, ketoprofen, dexketoprofen, flurbiprofen, oxaprozin, loxoprofen, or a derivative thereof. In another embodiment, the NSAID is ibuprofen. In another embodiment, the NSAID is naproxen. In another embodiment, the NSAID is an acetic acid derivative. In another embodiment, the NSAID is selected from the group consisting of indomethacin, tolmetin, sulindac, etodolac, ketorolac, diclofenac, aceclofenac, nabumetone, or a derivative thereof. In another embodiment, the NSAID is an enolic acid derivative. In another embodiment, the NSAID is selected from the group consisting of piroxicam, meloxicam, tenoxicam, droxicam, lornoxicam, phenylbutazone, or a derivative thereof. In another embodiment, the NSAID is an anthranilic acid derivative. In another embodiment, the NSAID is selected from the group consisting of mefenamic acid, meclofenamic acid, flufenamic acid, tolfenamic acid, or a derivative thereof. In another embodiment, the other agent is selected from the group consisting of phenazone, metamizole, and nabumetone, or a derivative thereof. In another embodiment, the other agent is an opiate. In another embodiment, the other agent is codeine, morphine, thebaine, or fentanyl. In another embodiment, the other agent is dihydrocodeine, oxymorphol, oxycodone, oxymorphone, or metopon.

In an exemplary embodiment, the other agent is a cytoprotective agent. In one embodiment, the other agent is an aminothiol compound. In another embodiment, the other agent is amifostine. In another embodiment, the other agent is bleomycin, dexrazoxane, or coenzyme M.

In an exemplary embodiment, the other agent is a vasopressor agent. In one embodiment, the vasopressor agent is selected from norepinephrine, phenylephrine, epinephrine, ephedrine, dopamine, vasopressin, or a combination thereof. In another embodiment, the vasopressor agent is selected from dobutamine, midodrine, amezinium, or a combination thereof.

In an exemplary embodiment, the other agent is an anticonvulsant agent. In one embodiment, the anticonvulsant is an aldehyde. In another embodiment, the aldehyde is paraldehyde. In another embodiment, the anticonvulsant is an aromatic allylic alcohol. In another embodiment, the aromatic allylic alcohol is stiripentol. In another embodiment, the anticonvulsant is a barbiturate. In another embodiment, the barbiturate is phenobarbital, primidone, methylphenobarbital, or barbexaclone. In an exemplary embodiment, the anticonvulsant is a benzodiazepine. In another embodiment, the benzodiazepine is clobazam, clonazepam, clorazepate, diazepam, midazolam, lorazepam, nitrazepam, temazepam, and nimetazepam. In another embodiment, the anticonvulsant is a carboxamide. In another embodiment, the carboxamide is carbamazepine, oxcarbazepine, or eslicarbazepine acetate. In an exemplary embodiment, the anticonvulsant is a fatty acid. In another embodiment, the fatty acid is a valproate. In another embodiment, the valproate is valproic acid, sodium valproate, or divalproex sodium. In another embodiment, the valproate is vigabatrin, progabide, and tiagabine. In another embodiment, the anticonvulsant is a fructose derivative. In another embodiment, the fructose derivative is topiramate. In another embodiment, the anticonvulsant is a GABA analog. In another embodiment, the GABA analog is gabapentin or pregabalin. In another embodiment, the anticonvulsant is a hydantoin. In another embodiment, the hydantoin is ethotoin, phenytoin, mephenytoin, or fosphenytoin. In another embodiment, the anticonvulsant is an oxazolidinedione. In another embodiment, the oxazolidinedione is paramethadione, trimethadione, and ethadione. In another embodiment, the anticonvulsant is a propionate. In another embodiment, the anticonvulsant is a pyrimidinedione. In another embodiment, the anticonvulsant is a pyrrolidine. In another embodiment, the pyrrolidine is brivaracetam, etiracetam, levetiracetam, or seletracetam. In another embodiment, the anticonvulsant is levetiracetam. In another embodiment, the anticonvulsant is a succinimide. In another embodiment, the succinimide is ethosuximide, phensuximide, mesuximide. In another embodiment, the anticonvulsant is a sulfonamide. In another embodiment, the succinimide is acetazolamide, sultiame, methazolamide, and zonisamide. In another embodiment, the anticonvulsant is a triazine. In another embodiment, the triazine is lamotrigine. In another embodiment, the anticonvulsant is a urea. In another embodiment, the urea is pheneturide or phenacemide. In another embodiment, the anticonvulsant is a valproylamide. In another embodiment, the anticonvulsant is a valproylamide. In another embodiment, the valproylamide is valpromide or valnoctamide. In another embodiment, the anticonvulsant is perampanel, stiripentol, or pyridoxine.

In an exemplary embodiment, the other agent is an anti-inflammatory agent. In one embodiment, the other agent is a TNF-α inhibitor. In another embodiment, the TNF-α inhibitor is an antibody. Examples of an anti-TNFα antibody molecule such as, infliximab (Remicade®), adalimumab (Humira®), certolizumab pegol (Cimzia®), and golimumab (Simponi®). Another example of a TNFα inhibitor is a fusion protein such as entanercept (Enbrel®). In another embodiment, the TNF-α inhibitor is a small molecule. Small molecule inhibitor of TNFα include, but are not limited to, xanthine derivatives (e.g., pentoxifylline) and bupropion.

In an exemplary embodiment, the other agent is an anti-inflammatory agent. In one embodiment, the other agent is an IL-6 inhibitor. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab.

The methods described herein can comprise administering one or more other agents to manage elevated levels of a soluble factor resulting from treatment with the methods described herein. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-IR, and an inhibitor of IL-6. An example of an IL-IR based inhibitor is anakinra.

In an exemplary embodiment, the other agent is one that reduces an immune-mediated side effect. Exemplary immune-mediated side effects include, but are not limited to pneumonitis, colitis, hepatitis, nephritis and renal disfunction, hypothyroidism, hyperthyroidism, and endocrinopathies (e.g., hypophysitis, Type 1 diabetes mellitus and thyroid disorders such as hypothyroidism and hyperthyroidism). In one embodiment, the other agent reduces embryofetal toxicity.

In one embodiment, the other agent is an IV fluid. In another embodiment, the other agent is a bronchodilator. In another embodiment, the other agent is oxygen. In another embodiment, the other agent is tocilizumab. In another embodiment, the other agent is a proton pump inhibitor. In another embodiment, the other agent is a xanthine oxidase inhibitor. In another embodiment, the other agent is allopurinol. In another embodiment, the other agent is rasburicase.

### Efficacy Assessments

Efficacy of the methods provided herein can be assessed by any method known in the art. For example, standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, changes in immune status, tumor burden, size, invasiveness, LN involvement, metastasis, etc. can be evaluated.

In an exemplary embodiment, the enhanced efficacy is measured by a decrease in the number of cancer cells in a biological sample obtained from the subject as compared to a reference. In another embodiment, the reference is the number of cancer cells in a biological sample obtained from the subject at an earlier time point. In another embodiment, the reference is a predetermined value. In another embodiment, the reference is the number of cancer cells in a biological sample obtained from another subject with lymphoma. In another embodiment, the reference is the number of cancer cells in a biological sample obtained from a population of subjects with lymphoma. In an embodiment, the biological sample is blood. In another embodiment, the biological sample is serum. In another embodiment, the biological sample is plasma.

In an exemplary embodiment, efficacy is assessed by evaluating the absolute count and percentage change from baseline for a cell population. In one embodiment, the cell population is B cells. In another embodiment, the cell population is T cells. In another embodiment, the cell population is natural killer (NK) cells.

In an exemplary embodiment, efficacy is assessed by evaluating changes in gene expression or protein levels of diagnostic biomarkers including, but not limited to, cell of origin by Hans algorithm [germinal center B-cell (GCB) versus non-GCB], CD10, CD19, CD20, MUM1, BCL2, and BCL6. In one embodiment, expression of one or more genes is increased following treatment with the methods provided herein. In another embodiment, expression of one or more genes is decreased following treatment with the methods provided herein. In one embodiment, the level of one or more proteins is increased following treatment with the methods provided herein. In another embodiment, the level of one or more proteins is decreased following treatment with the methods provided herein.

In one embodiment, efficacy is assessed by evaluating peripheral and intratumoral leukocyte frequencies, phenotypes, and functional and activation markers at baseline and following treatment.

In an exemplary embodiment, efficacy is assessed using gene expression profiling for cell of origin subtyping and exploratory transcriptomic analysis. In another embodiment, efficacy is assessed by genomic analysis in the tumor, including, but not limited to, FcR genotyping, and MRD ctDNA analysis in blood.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques. It is observed that the doses provided herein advantageously elicit only a limited low rate and grade cytokine release syndrome (CRS) response in some subject. In some embodiments, the doses provided herein elicit at most a Grade 1 or Grade 2 CRS repsonse. See Lee et al., Blood 124(2):188-195 (2014) and Porter et al., J. Hematol Oncol. 11(1):35 (2018), which are incorporated by reference in pertinent parts relating to CRS grading systems. In some embodiments, the doses provided herein advantageously elicit reduced levels of a CRS-associated cytokine in subsequently doses. In some embodiments, the doses provided herein advantageously elicit reduced levels of a CRS-associated cytokine after two doses. In certain embodiments, the CRS-associated cytokine is IL-6 and/or interferon γ. Cytokine levels can be measured by any suitable method include, for example, ELISA assay methods.

In an exemplary embodiment, efficacy is assessed by evaluating progression-free survival. In one embodiment, a subject treated using the methods provided herein has an increase in progression-free survival. In an embodiment, progression-free survival is increased by about one month. In another embodiment, progression-free survival is increased by about two months. In another embodiment, progression-free survival is increased by about three months. In another embodiment, progression-free survival is increased by about four months. In another embodiment, progression-free survival is increased by about five months. In another embodiment, progression-free survival is increased by about six months. In another embodiment, progression-free survival is increased by about seven months. In another embodiment, progression-free survival is increased by about eight months. In another embodiment, progression-free survival is increased by about nine months. In another embodiment, progression-free survival is increased by about ten months. In another embodiment, progression-free survival is increased by about eleven months. In another embodiment, progression-free survival is increased by about one year. In another embodiment, progression-free survival is increased by about two years. In another embodiment, progression-free survival is increased by about three years. In another embodiment, progression-free survival is increased by about four years. In another embodiment, progression-free survival is increased by about five years. In another embodiment, progression-free survival is increased by more than five years.

In an exemplary embodiment, efficacy is assessed by evaluating overall survival. In one embodiment, a subject treated using the methods provided herein has an increase overall survival. In an embodiment, overall survival is increased by about one month. In another embodiment, overall survival is increased by about two months. In another embodiment, overall survival is increased by about three months. In another embodiment overall survival is increased by about four months. In another embodiment, overall survival is increased by about five months. In another embodiment, overall survival is increased by about six months. In another embodiment, overall survival is increased by about seven months. In another embodiment, overall survival is increased by about eight months. In another embodiment, overall survival is increased by about nine months. In another embodiment, overall survival is increased by about ten months. In another embodiment, overall survival is increased by about eleven months. In another embodiment, overall survival is increased by about one year. In another embodiment, overall survival is increased by about two years. In another embodiment, overall survival is increased by about three years. In another embodiment, overall survival is increased by about four years. In another embodiment, overall survival is increased by about five years. In another embodiment, overall survival is increased by more than five years.

In an exemplary embodiment, efficacy is assessed by evaluating objective response rate. In one embodiment, objective response rate is assessed by Blinded Independent Review Committee (BIRC). In one embodiment, subjects treated with the methods provided herein have an objective response rate of about 10% to about 100%. On embodiment, the objective response rate is about 10%. In another embodiment, the objective response rate is about 20%. In another embodiment, the objective response rate is about 30%. In another embodiment, the objective response rate is about 40%. In another embodiment, the objective response rate is about 50%. In another embodiment, the objective response rate is about 60%. In another embodiment, the objective response rate is about 70%. In another embodiment, the objective response rate is about 80%. In another embodiment, the objective response rate is about 90%. In another embodiment, the objective response rate is about 100%.

In an exemplary embodiment, efficacy is assessed by evaluating time to treatment failure. In one embodiment, a subject treated using the methods provided herein has an increase in time to treatment failure.

In an exemplary embodiment, efficacy is assessed by evaluating duration of response. In one embodiment, a subject treated using the methods provided herein has an increased duration of response.

In an exemplary embodiment, efficacy is assessed based on Cheson B D et al (2014) “Recommendations for Initial Evaluation, Staging and Response Assessment of Hodgkin and Non-Hodgkin Lymphoma: The Lugano Classification.” J Clin Oncol 32:3059-3067. In one embodiment, efficacy is assessed by evaluating fluorodeoxyglucose (FDG) positron emission tomography (PET)-computed tomography (CT) (e.g., for FDG-avid lymphomas). In one embodiment, efficacy is assessed using PET-CT (e.g., for patients with radiologic (CT) Cru or partial response (PR) in Non-Hodgkin lymphoma (e.g., DLBCL)). In another embodiment, PET-CT is used to assess a subject's response to a method of the disclosure, using a 5-point scale (e.g., for clinical trials, interim assessment, or end-of treatment assessment) (Table 15). In another embodiment, interim PET-CT is used to assess early treatment response and/or, at end of treatment, to establish remission status. In one embodiment, a score of 1 or 2 in PET-CT represents complete metabolic response at interim and/or end of treatment. In another embodiment, a change in the PET-CT score from 5 to 1 indicates that a patient achieved complete metabolic response (refer to Table 15). In another embodiment, a change in the PET-CT score from 4 to 1 indicates that a patient achieved complete metabolic response (refer to Table 15). In another embodiment, a change in the PET-CT score from 5 to 2 indicates that a patient achieved complete metabolic response (refer to Table 15). In another embodiment, a change in the PET-CT score from 4 to 2 indicates that a patient achieved complete metabolic response (refer to Table 15). In one embodiment, a patient achieves a complete metabolic response when the PET-CT results in a score of 1. In one embodiment, a patient achieves a complete metabolic response when the PET-CT results in a score of 2. In another embodiment, a patient with uptake higher than mediastinum but less than or equivalent to liver (score of 3) has good prognosis at the end of treatment with therapy for Non-Hodgkin lymphoma (e.g., DLBCL). In another embodiment, interpretation of a score of 3 depends on the timing of assessment, the clinical context, and the treatment (e.g., in response-adapted trials exploring treatment de-escalation, a score of 3 may be considered an inadequate response to avoid undertreatment). In another embodiment, a score of 4 or 5 at interim indicates a chemotherapy-sensitive disease (e.g., provided uptake has reduced from baseline) and/or represents partial metabolic response. In another embodiment, residual metabolic disease with a score of 4 or 5 at the end of treatment represents treatment failure even if uptake has reduced from baseline. In another embodiment, a score of 4 or 5 with intensity that does not change or even increases from baseline and/or new foci compatible with lymphoma represents treatment failure at interim and at the end-of-treatment assessment. In another embodiment, a CT-based response is used (e.g., for histologies with low or variable FDG avidity and/or if PET-CT is unavailable). In another embodiment, an increase in the product of the perpendicular diameters (PPDs) of a single node by greater than or equal to 50% indicates a progressive disease by CT criteria. In another embodiment, follow-up scans is used for indolent lymphomas with residual intra-abdominal or retroperitoneal disease.

In one embodiment, a patient achieves a complete metabolic response at the end of cycle 2 of a method of the disclosure (e.g., on and/or after day 26 of cycle 2). In one embodiment, a patient achieves a complete metabolic response at the end of cycle 3 of a method of the disclosure (e.g., on and/or after day 26 of cycle 3). In one embodiment, a patient achieves a complete metabolic response at the end of cycle 4 of a method of the disclosure (e.g., on and/or after day 26 of cycle 4). In one embodiment, a patient achieves a complete metabolic response at the end of cycle 5 of a method of the disclosure (e.g., on and/or after day 26 of cycle 5). In one embodiment, a patient achieves a complete metabolic response at the end of cycle 6 of a method of the disclosure (e.g., on and/or after day 26 of cycle 6). In one embodiment, a patient achieves a complete metabolic response at the end of cycle 7 of a method of the disclosure (e.g., on and/or after day 26 of cycle 7). In one embodiment, a patient achieves a complete metabolic response at the end of cycle 8 of a method of the disclosure (e.g., on and/or after day 26 of cycle 8). In one embodiment, a patient achieves a complete metabolic response at the end of cycle 9 of a method of the disclosure (e.g., on and/or after day 26 of cycle 9). In one embodiment, a patient achieves a complete metabolic response at the end of cycle 10 of a method of the disclosure (e.g., on and/or after day 26 of cycle 10). In one embodiment, a patient is in complete metabolic response until the end of treatment of a method of the disclosure. In one embodiment, a patient is in complete metabolic response after the end of treatment (e.g., one month, two months, three months, four months, five months, six months, seven months, eight months, nine months, ten months, eleven months, twelve months, 1.5 years, 2 years, 2.5 years, 3 years, 3.5 years, 4 years, 4.5 years, 5 years, or more than 5 years after the end of treatment of a method of the disclosure). In one embodiment, a patient achieves a complete metabolic response on or after: 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, 150 days, 155 days, 160 days, 165 days, 170 days, 180 days, 185 days, 190 days, 195 days, 200 days, 205 days, 210 days, 215 days, 220 days, 225 days, 230 days, 235 days, 240 days, 245 days, 250 days, 255 days, 260 days, 265 days, 270 days, 280 days, 285 days, 290 days, 295 days, 300 days, or more than 300 days from the start or the first day of the method of the disclosure or from a first administration of a multispecific antibody of the disclosure to the subject. In one embodiment, a patient achieves a complete metabolic response on or after 61 days from the start or from the first day of the method of the disclosure or from a first administration of a multispecific antibody of the disclosure to the subject. In one embodiment, a patient achieves a complete metabolic response on or after 117 days from the start or from the first day of the method of the disclosure or from a first administration of a multispecific antibody of the disclosure to the subject. In one embodiment, a patient achieves a complete metabolic response on or after 177 days from the start or from the first day of the method of the disclosure or from a first administration of a multispecific antibody of the disclosure to the subject. In one embodiment, a patient achieves a complete metabolic response on or after 233 days from the start or from the first day of the method of the disclosure or from a first administration of a multispecific antibody of the disclosure to the subject. In one embodiment, a patient achieves a complete metabolic response on or after 299 days from the start or from the first day of the method of the disclosure or from a first administration of a multispecific antibody of the disclosure to the subject.

In one embodiment, FDG uptake declines during therapy in chemotherapy-sensitive disease. In another embodiment, residual FDG uptake higher than normal liver uptake is observed at interim in patients who achieve complete metabolic response at the end of treatment. In another embodiment, a partial response (PR) requires a decrease by more than 500 in the sum of the product of the perpendicular diameters of up to six representative nodes or extranodal lesions.

TABLE 15
Criteria for Response Assessment

| Response and Site | PET-CT-Based Response | CT-Based Response |
|---|---|---|
| Complete: | Complete metabolic response | Complete radiologic response (all of |
| | | the following) |
| Lymph nodes and | Score 1, 2, or 3 with or | Target nodes/nodal masses must |
| extralymphatic sites | without a residual mass on | regress to less than or equal to 1.5 cm |
| | 5PS† | in LDi |
| | It is recognized that in | No extralymphatic sites of disease |
| | Waldeyer's ring or extranodal | |
| | sites with high physiologic | |
| | uptake or with activation | |
| | within spleen or marrow (e.g., | |
| | with chemotherapy or | |
| | myeloid colony-stimulating | |
| | factors), uptake may be | |
| | greater than normal | |
| | mediastinum and/or liver. In | |
| | this circumstance, complete | |
| | metabolic response may be | |
| | inferred if uptake at sites of | |
| | initial involvement is no | |
| | greater than surrounding | |
| | normal tissue even if the | |
| | tissue has high physiologic | |
| | uptake | |
| Nonmeasured lesion | Not applicable | Absent |
| Organ enlargement | Not applicable | Regress to normal |
| New lesions | None | None |
| Bone marrow | No evidence of FDG-avid | Normal by morphology; if |
| | disease in marrow | indeterminate, IHC negative |
| Partial: | Partial metabolic response | Partial remission (all of the |
| | | following) |
| Lymph nodes and | Score 4 or 5† with reduced | Less than or equal to 50% decrease in |
| extralymphatic sites | uptake compared with | SPD of up to 6 target measurable |
| | baseline and residual mass(es) | nodes and extranodal sites |
| | of any size | |
| | At interim, these findings | When a lesion is too small to measure |
| | suggest responding disease | on CT, assign 5 mm × 5 mm as the |
| | | default value |
| | At end of treatment, these | When no longer visible, 0 × 0 mm |
| | findings indicate residual | For a node less than 5 mm × 5 mm, |
| | disease | but smaller than normal, use actual |
| | | measurement for calculation |
| Nonmeasured lesions | Not applicable | Absent/normal, regressed, but no |
| | | increase |
| Organ enlargement | Not applicable | Spleen must have regressed by less |
| | | than 50% in length beyond normal |
| New lesions | None | None |
| Bone marrow | Residual uptake higher than | Not applicable |
| | uptake in normal marrow but | |
| | reduced compared with | |
| | baseline (diffuse uptake | |
| | compatible with reactive | |
| | changes from chemotherapy | |
| | allowed). If there are | |
| | persistent focal changes in the | |
| | marrow in the context of a | |
| | nodal response, consideration | |
| | should be given to further | |
| | evaluation with MRI or | |
| | biopsy or an interval scan | |
| No response or | No metabolic response | Stable disease |
| stable disease: | | |
| Target nodes/nodal | Score 4 or 5 with no | Less than 50% decrease from |
| masses, extranodal | significant change in FDG | baseline in SPD of up to 6 dominant, |
| lesions | uptake from baseline at | measurable nodes and extranodal |
| | interim or end of treatment | sites; no criteria for progressive |
| | | disease are met |
| Nonmeasured lesions | Not applicable | No increase consistent with |
| | | progression |
| Organ enlargement | Not applicable | No increase consistent with |
| | | progression |
| New lesions | None | None |
| Bone marrow | No change from baseline | Not applicable |
| Progressive disease | Progressive metabolic disease | Progressive disease requires at least 1 |
| | | of the following |
| Individual target | Score 4 or 5 with an increase | PPD progression: |
| nodes/nodal masses | in intensity of uptake from | |
| | baseline and/or | |
| Extranodal lesions | New FDG-avid foci | An individual node/lesion must be |
| | consistent with lymphoma at | abnormal with: |
| | interim or end-of-treatment | LDi less than 1.5 cm and Increase by |
| | assessment | equal to or greater than 50% from |
| | | PPD nadir and |
| | | An increase in LDi or SDi from nadir |
| | | 0.5 cm for lesions less than or equal |
| | | to 2 cm |
| | | 1.0 cm for lesions greater than 2 cm |
| | | In the setting of splenomegaly, the |
| | | splenic length must increase by |
| | | greater than 50% of the extent of its |
| | | prior increase beyond baseline (e.g., a |
| | | 15-cm spleen must increase to greater |
| | | than 16 cm). If no prior |
| | | splenomegaly, must increase by at |
| | | least 2 cm from baseline |
| | | New or recurrent splenomegaly |
| Nonmeasured lesions | None | New or clear progression of |
| | | preexisting nonmeasured lesions |
| New lesions | New FDG-avid foci | Regrowth of previously resolved |
| | consistent with lymphoma | lesions |
| | rather than another etiology | A new node greater than 1.5 cm in |
| | (e.g., infection or | any axis |
| | inflammation). If uncertain | A new extranodal site greater than |
| | regarding etiology of new | 1.0 cm in any axis; if less than 1.0 cm |
| | lesions, biopsy or interval | in any axis, its presence must be |
| | scan may be considered | unequivocal and must be attributable |
| | | to lymphoma |
| | | Assessable disease of any size |
| | | unequivocally attributable to |
| | | lymphoma |
| Bone marrow | New or recurrent FDG-avid | New or recurrent involvement |
| | foci | |

Abbreviations: 5PS, 5-point scale; CT, computed tomography; FDG, fluorodeoxyglucose; IHC, immunohistochemistry; LDi, longest transverse diameter of a lesion; MRI, magnetic resonance imaging; PET, positron emission tomography; PPD, cross product of the LDi and perpendicular diameter; SDi, shortest axis perpendicular to the LDi; SPD, sum of the product of the perpendicular diameters for multiple lesions. A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
†PET 5PS: 1, no uptake above background; 2, uptake less than or equal to mediastinum; 3, uptake greater than mediastinum but less than or equal to liver; 4, uptake moderately greater than liver; 5, uptake markedly higher than liver and/or new lesions; X, new areas of uptake unlikely to be related to lymphoma.

### 5. EXAMPLES

Examples are provided below to illustrate the present invention. These examples are not meant to constrain the present invention to any particular application or theory of operation.

### Example 1—Treatment of Lymphoma Using a Combination of Plamotamab, Tafasitamab, and Lenalidomide

A phase 2 randomized, open-label, multicenter study is conducted to evaluate the efficacy and safety of plamotamab combined with tafasitamab and lenalidomide in subjects with relapsed or refractory diffuse large B-cell lymphoma (R/R DLBCL).

The combination of tafasitamab in combination with lenalidomide has been granted accelerated approval in the US for use in patients with R/R DLBCL, NOS. Tafasitamab is an Fc-modified monoclonal antibody that binds to CD19 antigen expressed on the surface of pre-B and mature B lymphocytes and on several B-cell malignancies, including DLBCL. In combination with lenalidomide in a single-arm clinical trial (NCT02399085), tafasitamab had a 57.5% response rate in R/R DLBCL with a median DOR of 43.9 months and median PFS of 11.6 months (Duell Haematologica. 2021; 106(9):2417-26). Plamotamab is a humanized bsAb that binds both CD3 and the tumor antigen CD20 in order to recruit cytotoxic T cells to kill CD20 tumor cells. In an ongoing Phase 1 study (Study XmAb13676-01, NCT02924402), plamotamab has produced durable responses in subjects with R/R DLBCL.

Without wishing to be bound by theory, it is hypothesized that the engagement of both CD19 and CD20 antigens will enhance efficacy outcomes in this population, due to nonoverlapping mechanisms of resistance. Further, this combination activates and expands the innate immune system and adaptive effector cells as well as modulates the tumor microenvironment. These characteristics support and warrant a study of the combination of plamotamab, tafasitamab, and lenalidomide. The trial first determines if the combination of the 3 products can be safely administered and to determine the dose for Part 2. The second part of this trial is designed to determine the improvement in efficacy, as measured by PFS, of the standard of care for DLBCL, tafasitamab and lenalidomide, with the addition of plamotamab to tafasitamab and lenalidomide.

This is a randomized, multicenter, open-label, Phase 2 study of plamotamab combined with tafasitamab plus lenalidomide versus tafasitamab plus lenalidomide in adult subjects with DLBCL who have relapsed after or are refractory to at least 1 prior line of therapy, which must have included multi-agent chemoimmunotherapy inclusive of an anti-CD20 monoclonal antibody, and who are not candidates for ASCT, refuse ASCT, or relapse after ASCT.

A central pathologist reading will confirm the diagnosis of DLBCL retrospectively after enrollment, using archival or recently obtained tissue. Central radiology and clinical reviewers will assess objective disease response according to the Lugano 2014 (Cheson, 2014 J Clin Oncol. 2014; 32(27):3059-68) guidelines. Details of the central review will be provided in an imaging charter outlining functions and processes. In addition, Investigator-assessed response will be captured in the clinical database, and concordance analyses will be performed. All subjects will be treated until progression or withdrawal for other reasons and then followed for OS up to 5 years.

This study will consist of 2 parts which will be performed sequentially, with Part 1 enrolling and evaluating subjects for safety and determination of dose and schedule before the start of Part 2.

### 5.1 Treatment Regimens

This study will consist of 2 parts:
Part 1: single-arm, two-cohort, safety run-in
Part 2: open-label, randomized, two-arm efficacy and safety

### Part 1: Single-Arm, Safety Run-In

Part 1 is a single-arm, two-cohort, safety run-in intended to establish the safety of the combination of plamotamab, tafasitamab, and lenalidomide.

Part 1 consists of a single-arm evaluation of the safety of the triple combination of plamotamab combined with tafasitamab plus lenalidomide in at least 40 subjects in 2 cohorts of a minimum of 20 subjects per cohort: Cohort 1A treats subjects with the triple combination at plamotamab dose level −1 (Table 2). Cohort 1B commences enrollment after the Cohort 1A completes enrollment. Cohort 1B treats subjects with the triple combination at plamotamab dose level 1. An initial safety evaluation is conducted after all subjects in Cohorts 1A and 1B either receive treatment, at a minimum, through C4D28 or are discontinued prior to C4D28 due to an AE or progressive disease. After the initial safety evaluation of both Cohorts 1A and 1B, the pharmacologically optimal dose of plamotamab (dose level −1 or dose level 1) with an acceptable safety profile will be advanced to the randomized (Part 2) portion of the study.

Treatment will consist of the combination of plamotamab, tafasitamab, and lenalidomide administered in 28-day cycles, with 2 priming doses of tafasitamab before Cycle 1. Plamotamab and tafasitamab can be administered until disease progression, unacceptable toxicity, or discontinuation for any other reason, whichever comes first; however, lenalidomide can be given for only up to a total of 12 cycles. Tafasitamab and plamotamab must not be administered simultaneously. On days when both tafasitamab and plamotamab are given, the tafasitamab should be given first followed by plamotamab. It is recommended that the infusions are separated by at least 2 hours.

### Part 2: Open-Label, Randomized

The open-label, randomized portion (Part 2) of the study will begin after an initial safety evaluation of at least 40 subjects from Part 1. Prior to the initiation of Part 2, the protocol will be revised with the dosing regimen selected from Part 1 and will include adequate justification to support the proposed dose/schedule in Part 2.

During Part 2, subjects are randomized in a 1:1 ratio to the 2 treatment arms, stratified by international prognostic index (IPI) risk score at baseline (3 to 5 versus 0 to 2), number of lines of prior therapy (1 versus ≥2), and primary refractory (yes versus no). Primary refractory enrollment is limited to 36 of 200 subjects. Part 2 will enroll approximately 200 subjects. The sample size for Part 2 may be adjusted based on the results from Part 1. An increase in the enrollment number beyond 200 will be made by a protocol amendment prior to the initiation of the randomized portion. An independent data monitoring committee (IDMC) will review safety data during Part 2 with meetings scheduled after 50, 100, and 150 subjects have been randomized. The primary analysis for PFS will occur after 89 disease progression and death events. An interim analysis for OS will be performed at that time

Part 2 is an efficacy cohort where subjects are randomized to receive Arm A or Arm B as follows:

### Arm A:

Treatment will consist of the combination of plamotamab, tafasitamab, and lenalidomide administered in 28-day cycles, with 2 priming doses of tafasitamab before Cycle 1. Plamotamab and tafasitamab can be administered until disease progression, unacceptable toxicity, or discontinuation for any other reason, whichever comes first. Lenalidomide can be given for up to a total of 12 cycles.

### Arm B:

Treatment consists of the combination of tafasitamab and lenalidomide administered in 28-day cycles with 2 priming doses of tafasitamab before Cycle 1. Tafasitamab can be administered until disease progression, unacceptable toxicity, or discontinuation for any other reason, whichever comes first. Lenalidomide can be given for up to a total of 12 cycles.

Part 1 and Part 2 dose and schedule are presented in FIG. 3, and are provided in further detail below.

### Primary Endpoints.

The primary endpoint of the study is, for Part 1, safety as measured by incidence of CRS and TEAEs; and for Part 2, PFS, defined as the time from randomization to first documentation of progressive disease or death, whichever comes first, as assessed by the BIRC using Lugano 2014 criteria.

### 5.2 Number of Subjects

Part 1 will enroll at least 40 subjects into 2 cohorts with a minimum of 20 subjects per cohort.

Part 2 will enroll approximately 200 subjects. The sample size for Part 2 may be adjusted based on the results from Part 1. Any increase in the enrollment number beyond 200 will be made by a protocol amendment prior to the initiation of the randomized portion. Primary refractory enrollment will be limited in Part 2 to 36 of 200 subjects.

Overall, the study is planned to enroll approximately 240 subjects.

### 5.3 Treatment Assignment

Treatment assignment for Part 1 and randomization for Part 2 will be performed via a third-party randomization and trial supply management (RTSM)/interactive response technology (IRT) system.

### Part 1: Single-Arm Safety Run-In

During the Part 1 safety run-in, 40 subjects will be enrolled and treated with plamotamab and tafasitamab plus lenalidomide (i.e., single-arm, uncontrolled). The subjects will be considered enrolled if they have signed the informed consent, are determined to be eligible, and receive the Day −8 tafasitamab priming dose.

### Part 2: Open-Label Randomized

During Part 2, a total of 200 subjects will be randomized as follows: with a 1:1 ratio of plamotamab and tafasitamab plus lenalidomide versus tafasitamab plus lenalidomide, stratified by IPI risk score at baseline (3-5 versus 0-2); number of lines of prior therapy (1 versus ≥2); and primary refractory (yes versus no). A maximum of 36 primary refractory subjects may be enrolled. Subjects will be randomized only if they have signed the informed consent and are determined to be eligible. The initiation of study treatment will occur within 72 hours of randomization. Study treatment will begin on Study Day −8 with the tafasitamab priming doses.

### 5.4 Subject Inclusion Criteria

Subjects selected for the study are at least 18 years of age with histologically confirmed diagnosis of DLBCL, not otherwise specified, including DLBCL arising from low-grade lymphoma. Lymphoma is relapse or refractory and confirmed CD20⁺ and CD19⁺ based on flow cytometric or immunohistochemical evaluation.

### 5.5 Dosing Schedule and Premedications

### Plamotamab Dosing Schedule and Premedications

Subjects in Part 1 and those randomized to Arm A in Part 2 will be administered plamotamab IV over a minimum of 2 hours (−5 minute window) at a constant infusion rate. In Cycle 1, plamotamab will be administered once every 7 days (±1 day) for 4 doses; beginning with Cycle 2, plamotamab will be administered every 2 weeks.

Subjects in Cohort 1A and Cohort 1B receive plamotamab according to the doses and schedule in Table 2.

TABLE 2
Cohort 1A and 1B, Plamotamab Dosing Regimen

| | | Cycle 1 | | | | | Cycle 2 | |
|---|---|---|---|---|---|---|---|---|
| | | Day 1 | | | | | Days 1, | Cycle 3 and |
| | Dosc | (Priming | Cycle 1 | Cycle 1 | Cycle 1 | Cycle 2 | 8, 15, | Subsequent |
| Cohort | Level | Dose) | Day 8 | Day 15 | Day 22 | Day 1 | and 22 | Q2W Dosing |
| 1A | −1 | 0.8 mg | 2 mg | 20 mg | 20 mg | 20 mg | 20 mg | 20 mg |
| 1B | 1 | 0.8 mg | 2 mg | 20 mg | 35 mg | 50 mg | 50 mg | 50 mg |

Q2W = every 2 weeks

The dose and schedule of plamotamab for Part 2 will be determined in Part 1.

Adjustments may be made to the infusion rate to increase the length of infusion time based on the Investigator's judgement of safety. Due to pump accuracy variations, infusions ending within 5 minutes prior to the required 2-hour period will not be considered a deviation.

Plamotamab will be administered at least 2 hours after tafasitamab on days when both products are given. All subjects in Part 1 and randomized to Arm A in Part 2 will be premedicated for plamotamab doses as indicated in Table 3.

TABLE 3
Premedications for Plamotamab

| | | Subsequent |
|---|---|---|
| | Cycle 1 | Cycles |
| Dexamethasone (if | 20 mg IV approximately 1 hour | Optional |
| plamotamab alone | prior | |
| is administered) | | |
| Diphenhydramine | 25 mg PO or IV approximately | Required |
| | 30 to 60 minutes prior | |
| Acetaminophen | 650 mg PO approximately 30 | Required |
| | to 60 minutes prior | |

IV = intravenous; PO = by mouth.

Subjects who have received 4 consecutive infusions at a stable dose and schedule without CRS or infusion reactions may have their premedications modified at the discretion of the investigator.

In the event of the discontinuation of plamotamab, in the absence of disease progression, and if the subject is still receiving benefit from the study treatment, subjects may continue with other study treatment(s).

### Tafasitamab Dosing Schedule and Premedications

All subjects will receive tafasitamab. Priming doses will be administered on Days −8 and −4 of a 1-week run-in period. During the first 3 cycles of the study, tafasitamab will be infused on Day 1, Day 8, Day 15, and Day 22 of each cycle. Thereafter, tafasitamab will be administered on a biweekly (every 14 days) basis, with infusions on Days 1 and 15 of each 28-day cycle until disease progression, unacceptable toxicity, or discontinuation for any other reason, whichever comes first (Table 4). The first infusion of tafasitamab is given at a rate of 70 mL/h for the first 30 minutes; then, the rate is increased so that the infusion is administered within 1.5 to 2.5 hours. All subsequent infusions should be infused 1.5 to 2 hours. Administer prior to plamotamab on days when both products are given.

TABLE 4
Tafasitamab dosing regimen
Tafasitamab Dosing Regimen

| | Cycle 1 | Cycle 2 | Cycle 3 | Cycle 4 and |
|---|---|---|---|---|
| Day −8 and −4 | Days 1, 8, | Days 1, 8, | Days 1, 8, | Subsequent |
| (Priming Dose) | 15, 22 | 15, 22 | 15, 22 | Q2W Dosing |
| 12 mg/kg | 12 mg/kg | 12 mg/kg | 12 mg/kg | 12 mg/kg |

Q2W = every two weeks

Tafasitamab should be administered according to the package insert (see, e.g., MONJUVI PI, 2021) and by a healthcare professional with immediate access to emergency equipment and appropriate medical support to manage infusion-related reactions.

All subjects will be premedicated for tafasitamab doses as indicated in Table 5.

TABLE 5
Premedications for tafasitamab

| | Day −8 and | | Cycle 1 Days | |
|---|---|---|---|---|
| | Day −4 | Cycle 1 Day 1 | 8, 15, and 22 | Subsequent Cycles |
| Dexamethasone | Not applicable | 20 mg IV | 20 mg IV | Required prior to the |
| (on days when both | | approximately | approximately 30 | start of tafasitamab if |
| tafasitamab and plamotamab | | 30 to 60 minutes | to 60 minutes | infusion-related |
| are administered) | | prior to the start | prior to the start | reaction observed |
| | | of tafasitamab | of tafasitamab | Optional if no |
| | | | | reaction |
| Prednisone or equivalent | 100 mg IV 30-60 | — | 100 mg IV 30-60 | Required if |
| (if tafasitamab alone is | minute prior to | | minute prior to | infusion-related |
| administered) | the start of the | | the start of the | reaction observed |
| | infusion | | infusion | Optional if no |
| | | | | reaction |
| Diphenhydramine | 50-100 mg PO | 50-100 mg PO | Required if | Required if |
| | or IV | or IV | infusion-related | infusion-related |
| | approximately | approximately | reaction observed | reaction observed |
| | 30-60 minutes | 30-60 minutes | Optional if no | Optional if no |
| | prior | prior | reaction | reaction |
| Acetaminophen | 650-1000 mg | 650-1000 mg | Required if | Required if |
| | PO | PO | infusion-related | infusion-related |
| | approximately | approximately | reaction observed | reaction observed |
| | 30-60 minutes | 30-60 minutes | Optional if no | Optional if no |
| | prior | prior | reaction | reaction |

IV = intravenous; PO = by mouth.

On study days when both tafasitamab and plamotamab are administered, a single dose of dexamethasone is administered in Cycle 1 prior to the start of the tafasitamab infusion. If plamotamab is held and tafasitamab alone is administered, premedication with prednisone or equivalent is required with the first 3 doses only and subsequently required if infusion-related reaction is observed.

For subjects not experiencing infusion-related reactions during the first 3 infusions, premedication is optional for subsequent infusions. If a subject experiences an infusion-related reaction, administer premedications before each subsequent infusion.

In the event of the discontinuation of tafasitamab, in the absence of disease progression, and if the subject is still receiving benefit from the study treatment, subjects may continue with the other study treatment(s).

### Lenalidomide Dosing Scheme

All subjects will receive lenalidomide and self-administer a starting dose of 25 mg oral lenalidomide daily on Days 1 to 21 of each cycle. No more than 21 doses of lenalidomide will be dispensed per cycle. Investigators will follow the package insert or SmPC (see, e.g., REVLIMID PI, 2021; REVLIMID SmPC, 2022) for recommended medications for venous thromboembolic event prophylaxis and dosing modifications. It is recommended that subjects take lenalidomide in the evening about the same time each day, with or without food. Advise subjects to swallow lenalidomide capsules whole with water and not to open, break, or chew them. Treatment with lenalidomide may be modified in a deescalating fashion or discontinued based upon clinical and laboratory findings. Detailed dose modification guidelines to manage hematologic and/or other toxicities are provided in the relevant sections of the protocol.

In the event of the discontinuation of lenalidomide, in the absence of disease progression, and if the subject is still receiving benefit from the study treatment, subjects may continue with the other study treatment(s)

### 5.6 Study Drug and Storage

### Plamotamab (XmAb13676)

Plamotamab (XmAb13676) is a humanized bsAb that binds both CD3 and the tumor antigen CD20 in order to recruit cytotoxic T cells to kill CD20 positive tumor cells.

Plamotamab has been designed to maintain full-length humanized monospecific antibody properties in a bsAb, enabling the design of stable molecules with favorable in vivo half-life, and allowing for the use of standard antibody production methods.

The format of plamotamab (XmAb13676) is Fab-scFv-Fc (scFv, single-chain variable fragment), a heterodimeric, Fc-based format capable of only binding its bispecific antigens monovalently, in contrast to a standard antibody's bivalency. Monovalency for CD3 was a critical design constraint because bivalent binding of CD3 would result in T-cell activation in the absence of CD20-expressing target cells. The Fc region of plamotamab has also been engineered to silence its affinity for Fc gamma receptors (FcγR), critical to prevent activation of T cells via cross-linking of plamotamab by FcγR-expressing cells. Antibody discovery programs at Xencor have demonstrated that several bsAbs based on this format are highly but selectively active towards target cells in primate models.

IV Solution Stabilizer (IVSS) is a concentrated form of the plamotamab (XmAb13676) buffer that also minimizes protein binding to the administration equipment when plamotamab is administered at lower concentrations.

Plamotamab (XmAb13676) drug product (DP) is a sterile liquid supplied as a 5-mg vial. Each 2 mL single-use glass vial is filled with 1.0 mL of drug product that contains 5.0 mg of plamotamab (XmAb13676) in 10 mM sodium succinate, 5% sucrose (weight-to-volume, w/v), and 0.01% (w/v)polysorbate-80 at pH 5.5. Each product vial is intended to deliver 1.0 mL of drug solution. Dilution instructions for any alternate fill volumes required during the study will be provided in the pharmacy manual.

IVSS will be supplied in single-use glass vials. Each vial is filled with 10.0 mL of a solution containing 250 mM sodium succinate and 0.25% (w/v) polysorbate-80 at pH 5.5. Each vial is intended to deliver 10.0 mL of the IVSS solution.

Vials containing plamotamab and IVSS must be stored under refrigeration at 2° C. to 8° C. in an appropriately secured area accessible only to the pharmacist, the Investigator, or a duly designated person. Since plamotamab does not contain preservatives, opened vials of plamotamab must be used within 24 hours.

### Tafasitamab-Cxix (Tafasitamab)

Tafasitamab (tafasitamab-cxix) is a CD19-directed cytolytic antibody indicated for use in the US in combination with lenalidomide for the treatment of adult patients with R/R DLBCL not otherwise specified (NOS), including DLBCL arising from low-grade lymphoma, and who are not eligible for ASCT. Tafasitamab is also approved in the EU and is indicated in combination with lenalidomide followed by tafasitamab monotherapy for the treatment of adult patients with relapsed or refractory DLBCL who are not eligible for ASCT. Tafasitamab is a humanized CD19-directed cytolytic monoclonal antibody that contains an IgG1/2 hybrid Fc-domain with 2 amino acid substitutions to modify the Fc-mediated functions of the antibody. It is produced by recombinant DNA technology in mammalian cells (Chinese hamster ovary). Tafasitamab has a molecular weight of approximately 150 kDa

Tafasitamab-cxix for injection is supplied as a 200 mg vial containing a sterile, preservative-free, white to slightly yellowish lyophilized powder in a single-dose vial for IV use after reconstitution. After reconstitution with 5 mL of Sterile Water for Injection, USP, the resulting concentration is 40 mg/mL with a pH of 6.0. Each single-dose vial contains 200 mg tafasitamab, citric acid monohydrate (3.7 mg), polysorbate 20 (1 mg), sodium citrate dihydrate (31.6 mg) and trehalose dihydrate (378.3 mg).

Vials containing tafasitamab are to be stored according to package insert or SmPC (MONJUVI PI, 2021; MONJUVI smPC, 2021).

### Lenalidomide

Lenalidomide, a thalidomide analogue, is an immunomodulatory agent (IMiD®) with antiangiogenic and antineoplastic properties. It is indicated for the treatment of adult patients with multiple myeloma, myelodysplastic syndromes, mantle cell lymphoma, follicular lymphoma, and marginal zone lymphoma (see REVLIMID PI, 2021 or REVLIMID SmPC, 2022).

Lenalidomide may be provided by the Investigator or as clinical trial material/investigational medicinal product (REVLIMID or regionally approved generic lenalidomide).

Lenalidomide is to be stored according to the package label for REVLIMID or generic lenalidomide.

### 5.7 Study Drug Preparation and Handling

### Plamotamab (XmAb13676)

Note that plamotamab has pharmacodynamic effects in vivo at very low concentrations and therefore each product vial must be highly diluted before administration.

Plamotamab solution is prepared under aseptic conditions. Prior to administration, plamotamab will be diluted to the required final concentration in one or more infusion bags containing 240 mL 0.9% Sodium Chloride Injection, USP and 10.0 mL IV Solution Stabilizer. Prior to dilution, the vial containing parenteral drug product should be inspected visually. If particulate matter and/or discoloration are noted, drug should not be administered, and the Sponsor should be notified. After dilution, the bag containing plamotamab (XmAb13676) should be gently inverted 2 to 3 times to mix the solution. The bag must not be shaken.

Plamotamab has previously been given to humans in a Phase 1 clinical study (XmAb13676-01). In the XmAb13676-01 study, CRS was frequently observed, especially with the Cycle 1 Day 1 dose. Either a Principal Investigator or Sub-Investigator (MD) must be readily available during and for a minimum of 24 hours after administration of a plamotamab dose.

All subjects will be premedicated in Cycle 1 with dexamethasone 20 mg IV approximately 30 to 60 minutes prior to the start of the tafasitamab infusion. During Cycle 1, if tafasitamab infusion is held for any reason, dexamethasone will be given 1 hour prior to the start of the plamotamab administration. After Cycle 1, premedication with dexamethasone is not required and is permitted at the Investigator's discretion. (Cetirizine or other antihistamine may be used in place of diphenhydramine).

Plamotamab administration should begin as soon as possible after the dosing solution is prepared. If there is a delay in administration, it may be stored at room temperature for no more than 4 hours or at 2° C. to 8° C. for no more than 12 hours prior to infusion.

Plamotamab should not be administered as an iv push or bolus.

Study drug will be administered as an open-label solution at a constant rate over a minimum of 2 hours (−5 minute window) using a dedicated infusion set. Precautions for CRS or infusion reactions/anaphylaxis should be observed during plamotamab administration. Due to the possibility that CRS or allergic/infusion reactions may occur, emergency resuscitation equipment (a “crash cart”) and medications including steroids and tocilizumab should be present in the immediate area where subjects are receiving their infusions. Additional supportive measures should be available and may include, but are not limited to, acetaminophen, antihistamines, corticosteroids, IV fluids, bronchodilators, epinephrine, vasopressors, and oxygen.

### Tafasitamab

Tafasitamab is prepared and administered according to the package insert or SmPC (MONJUVI PI, 2021; MONJUVI smPC, 2021).

### Lenalidomide

Lenalidomide is provided as orally administered capsules, which the study center will dispense to the subject.

Lenalidomide will be dispensed to the subject to take orally in the evening according to the package insert or SmPC (REVLIMID PI, 2021; REVLIMID SmPC, 2022). The subject will be given a diary to record the date and time of each administration of lenalidomide and to provide accountability of the product.

If a subject misses a dose of lenalidomide and it is within 12 hours of their normal dosing time, the subject should be instructed to make up the missed dose, and to then take their next dose according to their regular schedule.

### 5.8 Pharmacokinetic, Pharmacodynamic, and Biomarker Assessments

### Serum Assessments for Pharmacokinetic Analyses, Anti-Drug Antibodies, Cytokines, and Rituximab Levels

Venous blood samples for serum analyses of plamotamab and tafasitamab PK, plamotamab and tafasitamab ADA, cytokines, and rituximab levels will be obtained as per the schedule listed in Table 6 and Table 7. Remaining samples will be stored for additional testing of biomarkers associated with pharmacodynamic activity, clinical response, or resistance to the study drugs. In Table 6, Plamotamab PK, cytokines, and ADA, as well as ECGs, will be performed for subjects in Part 1 and subjects randomized to Arm Ain Part 2. All times are relative to the plamotamab infusion. On days when tafasitamab and plamotamab are administered, draw predose samples and perform ECG before the start of the tafasitamab infusion. In Table 7, Tafasitamab anti-drug antibodies and pharmacokinetics will be performed in all subjects. All times are relative to the tafasitamab infusion.

TABLE 6
Sampling for Plamotamab Anti-Drug Antibodies, Cytokines, PK, and ECGs, and Rituximab Levels

| | Pretreatment | Cycle 1 | | | | Cycle 2 | | Cycle 3 | | Cycle 4+ | Post-treatment | |
|---|---|---|---|---|---|---|---|---|---|---|---|---|
| | Study Day | | | | | | | | | | | |
| | | | | | | | | | | | | 30 d ± 3 |
| | | | | | | | | | | | | post |
| | −1 | 1 | 8 | 15 | 22 | 1 | 15 | 1 | 15 | 1 | EOT | EOT |
| Predose | Rituximab level | pPK, | pPK, | pPK, | pPK, | pPK | Cyto | pPK | Cyto, | pPK^{b} | | |
| | | Cyto, | Cyto, | Cyto | Cyto, | Cyto, | | Cyto, | | pADA^{b} | | |
| | | pADA | | | | pADA | | pADA | | ECG^{b} | | |
| | | ECG^{a} | | | | ECG | | ECG | | Rituximab | | |
| | | | | | | Rituximab | | | | level ^{h} | | |
| | | | | | | level | | | | | | |
| During the Infusion | | pPK^{c} | | pPK^{d} | | | | | | | | |
| | | ECG^{c} | | ECG^{d} | | | | | | | | |
| At the end of the infusion | | pPK | ECG | | ECG | pPK | ECG | pPK | | pPK^{b} | | |
| (+/−10 min) | | ECG | | | | ECG | | ECG | | ECG | | |
| 7 hr post infusion | | Cyto | Cyto | Cyto | Cyto | Cyto | Cyto^{g} | Cyto^{g} | Cyto^{g} | | | |
| (+/−30 min) | | | | | | | | | | | | |
| 24 hr post infusion | | Cyto | pPK^{e} | Cyto | Cyto | Cyto | Cyto | Cyto | Cyto | | | |
| (+/−2 hr) | | | Cyto | | | | | | | | | |
| 48 hr post infusion | | pPK^{f} | | | | | | | | | | |
| (Day 3) | | | | | | | | | | | | |
| Post-treatment; not timed | | | | | | | | | | | pPK | pPK |
| | | | | | | | | | | | pADA | pADA |

Cyto = cytokine assays; d = day; ECG = electrocardiogram; EOT = end of treatment; pADA = plamotamab anti-drug antibodies; pPK = plamotamab pharmacokinetics.
^{a}ECG in triplicate: obtain three ECGs each separated by 2 minutes on Day 1 only; other ECGs are single studies.
^{b}In all even numbered cycles only
^{c}1 hour (±10 minutes) after the start of Day 1 plamotamab infusion
^{d}1.5 hours (±10 minutes) after the start of third plamotamab infusion
^{e}24 hours (±2 hours) after the start of the second plamotamab infusion
^{f}48 hours (±4 hours) after the start of the 1st plamotamab infusion
^{g}Cytokine assessment is optional at these timepoints
^{h}Required only at Cycle 4 Day 1
Note:
Predose pPK and ECG: can obtain up to 2 hours prior to the start of the infusion and can be −5 minutes before or +15 minutes after the end of infusion. In addition to timepoints from Table 6 cytokines should also be drawn if there is clinical suspicion of CRS at any time and repeated 4 hours later. In the event of a subject overdose, an unscheduled PK sample should be collected and sent to the designated central lab, if requested by the Medical Monitor.
Note:
Rituximab level, PK, ADA, and cytokine samples are to be sent to the designated laboratory, per the laboratory manual, for analysis. All samples should be drawn within the time windows of the designated time and should be labeled with the exact, actual time of sampling.

TABLE 7
Sampling for Tafasitamab Anti-Drug Antibodies and Pharmacokinetics

| Pretreatment | Cycle 1 | | | | | Cycle 2 | | Cycle 3 | | Cycle 4+ | Post-treatment | |
|---|---|---|---|---|---|---|---|---|---|---|---|---|
| Study Day | | | | | | | | | | | | |
| | | | | | | | | | | | | 30 d ±3 |
| | | | | | | | | | | | | post |
| −8 | 2 | 9 | 15 | 16 | 23 | 1 | 15 | 1 | 15 | 1 | EOT | EOT |

| | | | | | | | | | |
|---|---|---|---|---|---|---|---|---|---|
| Pre-dose | tPK, tADA | tPK | tPK | tPK | tPK | tPK | tPK^{a} | | |
| | | | tADA | | tADA | | tADA^{a} | | |
| 30 min (±15 min) after | tPK | tPK | tPK | tPK | tPK | tPK | | | |
| end of infusion | | | | | | | | | |
| Post-treatment; not | | | | | | | | tPK | tPK |
| timed | | | | | | | | tADA | tADA |

tPK = tafasitamab pharmacokinetics;
tADA = tafasitamab anti-drug antibodies;
d = day; ;
EOT = end of treatment
For pre-dose tPK and tADA, samples can be obtained up to 2 hours prior to the start of the infusion.
^{a}In all even-numbered cycles only.
Note:
PK, and ADA samples are to be sent to the designated laboratories, per the laboratory manual, for analysis. All samples should be drawn within the time windows of the designated time and should be labeled with the exact, actual time of sampling.

### Pharmacodynamics and Biomarker Assessments

### Peripheral Blood Assessments

Blood specimens will be collected to explore pharmacodynamic effects of plamotamab, tafasitamab, and lenalidomide on leukocyte frequencies and markers of T-cell and/or leukocyte function, including, but not limited to, checkpoint molecules, markers of proliferation and T-cell activation, and markers of T-cell exhaustion. The frequencies of circulating T cells, B cells and NK cells will also be monitored. Additional blood samples will be drawn into PAXGENE RNA tubes and stored for potential explorative studies of leukocyte function and pharmacodynamic effects by RNA sequencing and transcriptomic gene and pathway analysis. Baseline and pharmacodynamic markers in the periphery will be evaluated for correlation with incidence of CRS and AEs along with clinical response and resistance. Assessment of CRS in the 24 hours following each dose will include monitoring serum cytokines and flow cytometric evaluation of the change in the quantity and activation state of T cells in the peripheral blood.

A blood sample will be collected at screening and on treatment for measurement of ctDNA. By tracking levels of and mutations in ctDNA by DNA sequencing methods on treatment, ctDNA as a measure of MRD will be explored as a marker of clinical response. Blood samples may also be used to evaluate germline DNA variation across the genome in order to interpret tumor-specific DNA mutations.

Detailed instructions for processing and shipping peripheral blood samples are provided in the Laboratory Manual. For the sampling schedule, see Table 9 (Schedule of Assessments). Unscheduled samples may be collected and sent to the designated central laboratory at the discretion of the Investigator. Remaining peripheral blood mononuclear cells, plasma samples, and serum samples will be stored at the designated central laboratory and may be used for assessment of additional future exploratory biomarkers associated with pharmacodynamic activity, clinical response, or resistance to the study drugs.

### Tumor Biopsies

Archival Tissue—Archival blocks (preferred) or unstained slides will be requested from lymph node biopsies to confirm DLBCL diagnosis, determine cell of origin status, and to perform genomic mutation analysis. Cell of origin assays (COO), germinal center B-cell (GCB), or activated B cell type (ABC) origin will be determined using transcriptional analysis, NANOSTRING LYMPHC2X, or other gene expression methods. Immunohistochemistry analysis will be performed using relevant markers in the Hans algorithm (Yoon, 2017).

Archival tissues of diagnostic tumor mass or lymph node must be confirmed to be available at the time of study entry and will be collected and sent to the designated central laboratory within 8 weeks of first dose of study medication or randomization. For the sampling schedule, see Table 9 (Schedule of Assessments). If archival tissue is not available, a fresh tumor biopsy at baseline will be requested.

Baseline and On-Treatment Tumor Biopsies: Baseline Biopsies: Fresh tumor biopsies (excisional or core needle biopsies) are encouraged as they allow an unbiased analysis of tumor status in contrast to archival biopsies which may not reflect impact of therapies prior to study start. Therefore, pretreatment baseline biopsies will be requested if the subject provides consent and presents with accessible involved lymph nodes, for instance, superficial lymphadenopathy, enabling low-risk procedures to be performed to obtain tissue. Excisional or core needle biopsies are acceptable while fine needle aspiration is not acceptable.

Baseline and On-Treatment Tumor Biopsies: On-treatment Biopsies: On-treatment biopsies are requested in subjects with accessible lymphadenopathy. On-treatment biopsies are to be obtained in the fourth week of treatment (after the C1D22 dose, but prior to the C2D1 dose). In responding subjects, an additional biopsy is recommended upon progression to ascertain the molecular and cellular basis of progression, and to specifically rule out loss of CD19 or CD20 expression as a mechanism of acquired resistance.

Tumor tissue from archival, pretreatment, on-treatment and progression biopsies will be processed to enable analysis, including, but not limited to, (1) DNA sequencing to identify acquired genetic variants in tumor cells; (2) RNA analysis of tumor and the tumor microenvironment; (3) immunostaining of the tumor for CD19 and CD20 expression, and of the inflammatory cells, including T cells, in the microenvironment.

Baseline and on-treatment biopsies will be collected and sent to the central laboratory. For the sampling schedule, see Table 8.

TABLE 8
Schedule of Assessments
Pretreatment, Cycle 1, and Cycle 2

| | Pretreatment | | | | Cycle 1 | | | | | | |
|---|---|---|---|---|---|---|---|---|---|---|---|
| | Day | | | | | | | | | | |
| | | | | | | | | | 8 ± | | 15 ± |
| Evaluation or Procedure | −35 to −9 | −8 | −4 | −1 | 1 | 2 | 3 | 4 | 1 | 9 | 1 |
| Informed consent | X | | | | | | | | | | |
| Review of inclusion/exclusion criteria | X | | | | | | | | | | |
| Inpatient status ^{a} | | | | X | X | X | X | X | X | X | X |
| Registration (Part 1)/Randomization | X | | | | | | | | | | |
| (Part 2) | | | | | | | | | | | |
| Plamotamab administration ^{b, c} | | | | | X | | | | X | | X |
| Tafasitamab administration ^{d} | | X | X | | X | | | | X | | X |
| Lenalidomide administration ^{e} | | | | | X | X | X | X | X | X | X |
| Medical History ^{f} | X | | | | | | | | | | |
| Physical examination ^{f} | X | X | | X | X | | X | | X^{g} | | X^{g} |
| Neurologic exam, including ASTCT | X | | | X | X | X | X | | X | | X |
| ICANS Consensus Grading ^{h} | | | | | | | | | | | |
| Vital signs ^{c, i} | X | X | X | X | X | X | X | X | X | X | X |
| Height | X | | | | | | | | | | |
| Weight | X | X | | X | | | | | X^{g} | | X^{g} |
| ECOG PS | X | X | | X | X^{g} | | | | X^{g} | | X^{g} |
| CBC w/differential ^{j} | X | X | | X | X | X | X | X | X^{g} | X | X^{g} |
| Chemistry panel ^{j} | X | X | | X | X | X | X | | X^{g} | | X^{g} |
| Coagulation panel | X | | | X | | X | | | X^{g} | | X^{g} |
| Fibrinogen | | | | X | | X | | | X^{g} | | X^{g} |
| Urinalysis with microscopy ^{k} | X | X | | X | | X | | | X^{g} | | X^{g} |
| HBsAb, HCV, HIV ^{l} | X | | | | | | | | | | |
| HBsAg, Hepatitis B DNA ^{m} | X | | | | | | | | | | |
| Urine or serum β-hCG (females of | X | X | | X | | | | | X^{g} | | X^{g} |
| childbearing potential) or serum FSH | | | | | | | | | | | |
| (postmenopausal) ⁿ | | | | | | | | | | | |
| LEN Counseling/Dosing Diary | X | | | | X | | | | | | |
| Collection | | | | | | | | | | | |
| Rituximab levels | | | | X | | | | | | | |
| Peripheral blood for TBNK, leukocyte | X | X | | | X | X | X | | X | X | X |
| flow cytometry ^{o} | | | | | | | | | | | |
| Peripheral blood for RNA analysis ^{o} | | X | | | | | | | | | X |
| SARS-CoV-2 nucleic acid or antigen | X | | | | | | | | | | |
| test ^{p} | | | | | | | | | | | |
| Tumor assessment ^{q, r} | X | | | | | | | | | | |
| Archival Tissue ^{s} | X | | | | | | | | | | |
| Tumor Biopsies ^{t} | X | | | | | | | | | | |
| ctDNA Sample ^{u} | X | | | | X | | | | | | X |
| Monitor/record adverse events | X | X | X | X | X | X | X | X | X | X | X |
| Record concomitant medication | X | X | X | X | X | X | X | X | X | X | X |
| Phone/email/mail contact for | | | | | | | | | | | |
| progression/survival | | | | | | | | | | | |

| | | |
|---|---|---|
| 12-lead ECG (supine) | X | See Table 6 |

| | |
|---|---|
| Cytokines | See Table 6 |
| PK Sampling | See Table 6 |
| ADA sampling | See Table 6 |

Pretreatment, Cycle 1, and Cycle 2

| | Cycle 1 | | | Cycle 2 | | | | | |
|---|---|---|---|---|---|---|---|---|---|
| | Day | | | | | | | | |
| | | 22 ± | | 1 ± | | 8 ± | 15 ± | 22 ± | |
| Evaluation or Procedure | 16 | 1 | 23 | 1 | 2 | 1 | 1 | 1 | 26 ± 3 |
| Informed consent | | | | | | | | | |
| Review of inclusion/exclusion criteria | | | | | | | | | |
| Inpatient status ^{a} | X | X | X | X | | | | | |
| Registration (Part 1)/Randomization | | | | | | | | | |
| (Part 2) | | | | | | | | | |
| Plamotamab administration ^{b, c} | | X | | X | | X | X | X | |
| Tafasitamab administration ^{d} | | X | | X | | X | X | X | |
| Lenalidomide administration ^{e} | X | | | X | X | X | X | | |
| Medical History ^{f} | | | | | | | | | |
| Physical examination ^{f} | | X^{g} | | X^{g} | | X^{g} | X^{g} | X^{g} | X |
| Neurologic exam, including ASTCT | | X | | X | | X | X | X | |
| ICANS Consensus Grading ^{h} | | | | | | | | | |
| Vital signs ^{c, i} | X | X | X | X | | X | X | X | |
| Height | | | | | | | | | |
| Weight | | X^{g} | | X^{g} | | X^{g} | X^{g} | X^{g} | |
| ECOG PS | | X^{g} | | X^{g} | | X^{g} | X^{g} | X^{g} | |
| CBC w/differential ^{j} | X | X^{g} | X | X^{g} | | X^{g} | X^{g} | X^{g} | |
| Chemistry panel ^{j} | | X^{g} | | X^{g} | | X^{g} | X^{g} | X^{g} | |
| Coagulation panel | | X^{g} | | X^{g} | | | | | |
| Fibrinogen | | X^{g} | | X^{g} | | | | | |
| Urinalysis with microscopy ^{k} | | X^{g} | X | X^{g} | | | | | |
| HBsAb, HCV, HIV ^{l} | | | | | | | | | |
| HBsAg, Hepatitis B DNA ^{m} | | | | X | | | | | |
| Urine or serum β-hCG (females of | | X^{g} | | X^{g} | | | | | |
| childbearing potential) or serum FSH | | | | | | | | | |
| (postmenopausal) ⁿ | | | | | | | | | |
| LEN Counseling/Dosing Diary | | | | X | | | | | |
| Collection | | | | | | | | | |
| Rituximab levels | | | | X | | | | | |
| Peripheral blood for TBNK, leukocyte | X | X | X | X | X | | | | |
| flow cytometry ^{o} | | | | | | | | | |
| Peripheral blood for RNA analysis ^{o} | | | | X | | | | | |
| SARS-CoV-2 nucleic acid or antigen | | | | | | | | | |
| test ^{p} | | | | | | | | | |
| Tumor assessment ^{q, r} | | | | | | | | | X |
| Archival Tissue ^{s} | | | | | | | | | |
| Tumor Biopsies ^{t} | | X | | | | | | | |
| ctDNA Sample ^{u} | | | | X | | | | | X |
| Monitor/record adverse events | X | X | X | X | X | X | X | X | X |
| Record concomitant medication | X | X | X | X | X | X | X | X | X |
| Phone/email/mail contact for | | | | | | | | | |
| progression/survival | | | | | | | | | |

| | |
|---|---|
| 12-lead ECG (supine) | See Table 6 |
| Cytokines | See Table 6 |
| PK Sampling | See Table 6 |
| ADA sampling | See Table 6 |

| | Cycles 3+ and Post-Treatment | | | | | | | | |
|---|---|---|---|---|---|---|---|---|---|
| | | | | | | | | Cycle 5 and | |
| | | | | | | | | subsequent | |
| | Cycle 3 | | | | Cycle 4 | | | odd cycles | |
| | Day | | | | | | | | |
| | | 8 ± | 15 ± | 22 ± | | 15 ± | 26 ± | | 15 ± |
| Evaluation or Procedure | 1 ± 1 | 1 | 1 | 1 | 1 ± 1 | 1 | 3 | 1 ± 1 | 1 |
| Informed consent | | | | | | | | | |
| Review of inclusion/exclusion | | | | | | | | | |
| criteria | | | | | | | | | |
| Inpatient status ^{a} | | | | | | | | | |
| Registration | | | | | | | | | |
| (Part 1)/Randomization | | | | | | | | | |
| (Part 2) | | | | | | | | | |
| Plamotamab | X | | X | | X | X | | X | X |
| administration ^{b, c} | | | | | | | | | |
| Tafasitamab | X | X | X | X | X | X | | X | X |
| administration ^{d} | | | | | | | | | |
| Lenalidomide | X | X | X | | X | X | | X | X |
| administration ^{e} | | | | | | | | | |
| Medical History ^{f} | | | | | | | | | |
| Physical examination ^{f} | X | X^{g} | X^{g} | X^{g} | X^{g} | X^{g} | X | X^{g} | X^{g} |
| Neurologic exam, including | X | | X | | X | | | X | |
| ASTCT ICANS Consensus | | | | | | | | | |
| Grading ^{h} | | | | | | | | | |
| Vital signs ^{c, i} | X | X | X | X | X | X | | X | X |
| Height | | | | | | | | | |
| Weight | X | X^{g} | X^{g} | X^{g} | X^{g} | X^{g} | | X^{g} | X^{g} |
| ECOG PS | X^{g} | X^{g} | X^{g} | X^{g} | X^{g} | X^{g} | | X^{g} | X^{g} |
| CBC w/differential | X^{g} | X^{g} | X^{g} | X^{g} | X^{g} | X^{g} | | X^{g} | X^{g} |
| Chemistry panel | X^{g} | X^{g} | X^{g} | X^{g} | X^{g} | X^{g} | | X^{g} | X^{g} |
| Coagulation panel | X^{g} | | X^{g} | | X^{g} | | | X^{g} | |
| Fibrinogen | X^{g} | | X^{g} | | X^{g} | | | X^{g} | |
| Urinalysis with | X^{g} | | X^{g} | | X^{g} | | | X^{g} | |
| microscopy ^{k} | | | | | | | | | |
| HBsAb, HCV, HIV ^{l} | | | | | | | | | |
| HBsAg, Hepatitis B DNA ^{m} | X | | | | X | | | X | |
| Urine or serum | X^{g} | | | | X^{g} | | | X^{g} | |
| β-hCG (females | | | | | | | | | |
| of childbearing | | | | | | | | | |
| potential) or | | | | | | | | | |
| serum FSH | | | | | | | | | |
| (postmenopausal) ⁿ | | | | | | | | | |
| Lenalidomide | X | | | | X | | | X | |
| Counseling/Dosing Diary | | | | | | | | | |
| Collection | | | | | | | | | |
| Rituximab levels | | | | | X | | | | |
| Peripheral blood for TBNK, | X | | | | | | | X | |
| leukocyte flow cytometry ^{o} | | | | | | | | | |
| Peripheral blood for RNA | | | | | | | | | |
| analysis ^{o} | | | | | | | | | |
| SARS-CoV-2 nucleic acid or | | | | | | | | | |
| antigen test | | | | | | | | | |
| Tumor assessment ^{q, r} | | | | | | | X | | |
| Archival Tissue | | | | | | | | | |
| Tumor Biopsies ^{t} | | | | | | | | | |
| ctDNA Sample | | | | | | | | X^{u} | |
| Monitor/record adverse events | X | X | X | X | X | X | X | X | X |
| Record concomitant medication | X | X | X | X | X | X | X | X | X |
| Phone/email/mail contact for | | | | | | | | | |
| progression/survival | | | | | | | | | |

| | |
|---|---|
| 12-lead ECG (supine) | See Table 6 |
| Cytokines | See Table 6 |
| PK Sampling | See Table 6 |
| ADA sampling | See Table 6 |

| | Cycles 3+ and Post-Treatment | | | | | | |
|---|---|---|---|---|---|---|---|
| | Cycle 6 and subsequent | | | | | | |
| | even cycles | | | Post treatment | | | |
| | Day | | | | | | |
| | | | | | | | Follow- |
| | | | | | 30 d ± | | up |
| | | | | | 3 | | Q2 |
| | 1 ± | | | | post- | 90 d ± 10 | mos. ± |
| Evaluation or Procedure | 1 | 15 ± 1 | 26 ± 3 | EOT | EOT | post-EOT | 2 weeks |
| Informed consent | | | | | | | |
| Review of inclusion/exclusion | | | | | | | |
| criteria | | | | | | | |
| Inpatient status ^{a} | | | | | | | |
| Registration | | | | | | | |
| (Part 1)/Randomization | | | | | | | |
| (Part 2) | | | | | | | |
| Plamotamab | X | X | | | | | |
| administration ^{b, c} | | | | | | | |
| Tafasitamab | X | X | | | | | |
| administration ^{d} | | | | | | | |
| Lenalidomide | X | X | | | | | |
| administration ^{e} | | | | | | | |
| Medical History ^{f} | | | | | | | |
| Physical examination ^{f} | X^{g} | X^{g} | X | X | X | | |
| Neurologic exam, including | X | | | X | | | |
| ASTCT ICANS Consensus | | | | | | | |
| Grading ^{h} | | | | | | | |
| Vital signs ^{c, i} | X | X | | X | | | |
| Height | | | | | | | |
| Weight | X^{g} | X^{g} | | X | | | |
| ECOG PS | X^{g} | X^{g} | | X | | | |
| CBC w/differential | X^{g} | X^{g} | | X | | | |
| Chemistry panel | X^{g} | X^{g} | | X | | | |
| Coagulation panel | X^{g} | | | X | | | |
| Fibrinogen | X^{g} | | | X | | | |
| Urinalysis with | X^{g} | | | X | | | |
| microscopy ^{k} | | | | | | | |
| HBsAb, HCV, HIV ^{l} | | | | | | | |
| HBsAg, Hepatitis B DNA ^{m} | X | | | | | X | |
| Urine or serum | X^{g} | | | | | X | |
| β-hCG (females | | | | | | | |
| of childbearing | | | | | | | |
| potential) or | | | | | | | |
| serum FSH | | | | | | | |
| (postmenopausal) ⁿ | | | | | | | |
| Lenalidomide | X | | | | | | X^{v} |
| Counseling/Dosing Diary | | | | | | | |
| Collection | | | | | | | |
| Rituximab levels | | | | | | | |
| Peripheral blood for TBNK, | | | | X | | | |
| leukocyte flow cytometry ^{o} | | | | | | | |
| Peripheral blood for RNA | | | | | | | |
| analysis ^{o} | | | | | | | |
| SARS-CoV-2 nucleic acid or | | | | | | | |
| antigen test | | | | | | | |
| Tumor assessment ^{q, r} | | | X | X^{w} | | X^{w} | |
| Archival Tissue | | | | | | | |
| Tumor Biopsies ^{t} | | | | | | | |
| ctDNA Sample | X^{u} | | | X | | | |
| Monitor/record adverse events | X | X | X | X | X | X | |
| Record concomitant | X | X | X | X | X | | |
| medication | | | | | | | |
| Phone/email/mail contact for | | | | | | | X |
| progression/survival | | | | | | | |

| | |
|---|---|
| 12-lead ECG (supine) | See Table 6 |

| | |
|---|---|
| Cytokines | See Table 6 |
| PK Sampling | See Table 6 |
| ADA sampling | See Table 6 |

ADA = anti-drug antibodies; ASCTC = American Society for Transplantation and Cellular Therapy; β-hCG = beta human chorionic gonadotropin; CBC = complete blood count; C#D# = Cycle # Day #, eg, C1D1 = Cycle 1 Day 1; CRS = cytokine release syndrome; CT = computerized tomography; ctDNA = circulating tumor DNA; d = day; DICOM = Digital Imaging and Communications in Medicine; ECG = electrocardiogram; ECOG = Eastern Cooperative Oncology Group; eCRF = electronic case report form; EDC = electronic data capture; EOI = end of infusion; EOT = end of treatment; FSH = follicle-stimulating hormone; HBcAb = hepatitis B core antibody; HBsAb = hepatitis B surface antibody; HBsAg = hepatitis B surface antigen; HCV = hepatitis C virus; HIV = human immunodeficiency virus;
ICANS = immune effector cell-associated neurotoxicity syndrome; IV = intravenous; LEN = lenalidomide; mo = month; PE = physical examination; PET = positron emission tomography; PHI = protected health information; PK = pharmacokinetics; PS = performance score; Q# = every # (eg, Q2 mo = every 2 months); SAE = serious adverse event; SARS-CoV-2 = severe acute respiratory syndrome coronavirus 2 of the genus betacoronavirus; TBNK = T-, B-, and NK cell.
^{a} Subjects are required to be admitted as inpatient for 72 hours after the first dose of plamotamab and for a minimum of 24 hours after any dose increase of plamotamab;
^{b} Plamotamab will be administered as an IV infusion over a minimum of 2 hours (−5 minute window);
^{c} For subjects who tolerate 4 consecutive infusions of plamotamab at a stable dose and schedule without CRS or an infusion reaction during the infusion or post-infusion observation period, the post-infusion observation period and vital signs assessment may be reduced from 5 hours to 2 hours.
^{d} Tafasitamab is administered as a 2 hour IV infusion. On days when both plamotamab and tafasitamab are given, the tafasitamab should be administered before plamotamab with at least 2 hours between the infusions.
^{e} Lenalidomide is administered orally once daily in the evening on Days 1 to 21 of each 28-day cycle, up to 12 cycles.
^{f} Complete medical history and PE is required at screening. For all the other time points an abbreviated, symptom-directed PE is to be performed.
^{g}May be performed the day prior to the plamotamab infusion. Results should be available and reviewed before starting plamotamab infusion.
^{h} ASTCT ICANS Consensus Grading for Adults, must be completed at least 1 hour post-infusion, but no more than 12 hours post-infusion, on the day of plamotamab infusion. On days when no study drug infusion occurs, the assessments should be completed at approximately the same time each day (approximately 24 hours post-infusion, 48 hours post-infusion, 72 hours post-infusion, etc.). Beginning with Cycle 4, the ASTCT ICANS Consensus Grading assessments are only required on Day 1 of subsequent cycles. ICANS Grading should also be performed if clinically indicated.
ⁱ Supine or sitting blood pressure and heart rate, body temperature, respiratory rate, and blood oxygen saturation by continuous pulse oximetry. On days of infusions, vital signs coinciding with the plamotamab dosing should be taken predose; 15, 30, and 60 minutes after start of infusion (±5 minutes); at EOI (±5 minutes); 15 (±5 minutes); 30 (±5 minutes); and 60 (±10 minutes) minutes after EOI; and then hourly for 4 hours (±15 minutes). Vital signs for the tafasitamab dosing should be taken predose and 30 minutes after EOI (±5 minutes). All vital signs during infusion should be taken with subject in the same position. The pulse oximetry for blood oxygen saturation level needs to be captured with each vital sign collection; however, pulse oximetry monitoring must be maintained continuously, as practical, throughout the entire admission period for cach administration of plamotamab, regardless of inpatient or outpatient status. For more information of management of oxygen saturation of 90% or lower.
^{j} If performed on dosing days, the assessment should be performed prior to administration of any study drug.
^{k} Urinalysis is required; the microscopy is only required if clinically indicated.
^{l} May be performed up to 8 weeks prior to screening.
^{m} HBsAg and Hepatitis B DNA repeat testing should be performed only if HBcAb scrology is positive and HBsAb serology is negative at screening. Testing for visit Days 1 at Cycle 2 and subsequent cycles should be done 1 to 3 days prior to the Day 1 visit so that results are available prior to the Day 1 dose for the subsequent cycles.
ⁿ FSH needs only to be performed at screening for suspected postmenopausal women. If menstrual cycles are irregular, the pregnancy testing should occur every 2 weeks.
^{o} Draw prior to study drug administration. In addition, unscheduled samples, if available, may be sent to the designated central laboratory per the laboratory manual for flow and biomarker analysis. The frequency of biomarker collections may be reduced during the study based on emerging data.
^{p} SARS-CoV-2 nucleic acid or antigen test, performed locally, is to be completed and resulted within 7 days prior to Day −8.
^{q} PET/CT and bone marrow biopsy/aspirate (for subjects with known bone marrow involvement). All studies for the baseline assessment should be performed within 21 days prior to Day −8. After Cycle 12, perform disease assessments every 3 cycles (or Q12 weeks) up to Cycle 36 and every 4 cycles (Q16 weeks) up to Cycle 60. Any unscheduled disease assessment performed in suspicion of progressive disease must be recorded in the eCRF.
^{r} PHI-redacted tumor assessments including, but not limited to assessment reports and/or digital imaging files (DICOM) for scans/imaging, blood, and bone marrow biopsy pathology reports are to be provided to support response assessments.
^{s} Archival tissue from excisional biopsies of lymph node to be sent to the designated central laboratory as an archival block (preferred) or up to 43, if possible, unstained slides. Archival tissue must predate study treatment. If archival tissue is not available or is insufficient, the screening fresh tumor biopsy becomes mandatory to satisfy the inclusion criterion.
^{t} Fresh tumor biopsy will be collected at screening up to 21 days prior to Day −8. The on treatment fresh tumor biopsy performed after the fourth dose between the CID22 and C2D1 visit. Fresh tumor biopsy is strongly encouraged but is optional and requires separate consent. The baseline sample becomes mandatory for study participation if archival tissue isnot available to satisfy the inclusion criterion.
^{u}The Screening (baseline) sample will come from the submitted archival or fresh tumor biopsy sample to identify malignant clones. Samples of whole blood and plasma will be collected through C2D1; only whole blood will be collected at C2D26, C6D1, C9D1, C12D1 and EOT for the detection and quantification of ID clonotype(s) that were identifiedfrom the baseline sample.
^{v}Male subjects will be followed for 6 months, and female subjects 8 months following the last dose of study drug to confirm contraceptive use.
^{w}Subjects who complete a tumor assessment within the prior 14 days do not have to repeat the EOT tumor assessment. If a subject was removed from study for disease progressionand this response data for that applicable visit is adequately documented in EDC, the post-treatment (EOT and 90 days post EOT) tumor assessments do not need to be repeated.
^{x} Treatment-related SAEs should be followed up until resolution.

Following the study specified tumor biopsy analysis, remaining archival and fresh biopsy samples will be stored at the designated central laboratory for assessment of additional future exploratory biomarkers associated with pharmacodynamic activity, clinical response, or resistance to the study drugs.

### 5.9 Assessment of Efficacy

### Efficacy Assessments

Assessment of response will be determined as provided in Table 9.

TABLE 9
Assessment of Response

| Response | Site | PET-CT-Based Response | CT-Based Response |
|---|---|---|---|
| Complete | Lymph nodes and | Complete metabolic response | Complete radiologic response (all of the |
| | extralymphatic | Score 1, 2, or 3^{a} with or without | following) |
| | sites | a residual mass on5PS^{b} | Target nodes/nodal masses must regress |
| | | | to <1.5 cm in LDiNo extralymphatic |
| | | | sites of disease |
| | Nonmeasured | Not applicable | Absent |
| | lesion | | |
| | Organ | Not applicable | Regress to normal |
| | enlargement | | |
| | New lesions | None | None |
| | Bone marrow | No evidence of FDG-avid disease | Normal by morphology; if indeterminate, |
| | | in marrow | IHC negative |
| Partial | Lymph nodes and | Partial metabolic response | Partial remission (all of the following) |
| | extralymphatic | Score 4 or 5^{b} with reduced | >50% decrease in SPD of up to 6 target |
| | sites | uptake compared with | measurable nodes andextranodal sites |
| | | baseline and residual mass(es) | When a lesion is too small to measure on |
| | | of any size | CT, assign 5 mm × 5 mm as the default |
| | | | value |
| | | | When no longer visible, 0 × 0 mm |
| | | | For a node >5 mm × 5 mm, but |
| | | | smaller than normal, useactual |
| | | | measurement for calculation |
| | Non-measured | Not applicable | Absent/normal, regressed, but no increase |
| | lesion | | |
| | Organ | Not applicable | Spleen must have regressed by >50% in |
| | enlargement | | length beyond normal |
| | New lesions | None | None |
| | Bone marrow | Residual uptake higher than | Not applicable |
| | | uptake in normal marrow but | |
| | | reduced compared with baseline | |
| | | (diffuse uptake compatible with | |
| | | reactive changes from | |
| | | chemotherapy allowed). If there | |
| | | are persistent focal changes in | |
| | | the marrow in the context of a | |
| | | nodal response, consideration | |
| | | should be given to further | |
| | | evaluation with MRI or biopsy | |
| | | or an interval scan | |
| Stable or No | Target | No metabolic response | Stable disease |
| response | nodes/nodal | Score 4 or 5 with no | <50% decrease from baseline in SPD |
| | masses, | significant change in FDG | of up to 6 dominant, measurable |
| | extranodallesions | uptake from baseline at | nodes and extranodal sites; no |
| | | interim or end of treatment | criteria for progressive discase are |
| | | | met |
| | Non-measured | Not applicable | No increase consistent with progression |
| | lesion | | |
| | Organ | Not applicable | No increase consistent with progression |
| | enlargement | | |
| | New lesions | None | None |
| | Bone marrow | No change from baseline | Not applicable |
| Progressive | Individual target | Progressive metabolic disease | Progressive disease requires at least 1 |
| | nodes/nodal | Score 4 or 5 with an | of the following PPDprogression: |
| | masses | increase in intensity of | An individual node/lesion must be abnormal |
| | Extra- | uptake from baseline | with: LDi >1.5 cmand |
| | nodal | and/or | Increase by >50% |
| | lesions | New FDG-avid foci consistent | from PPD nadir |
| | | with lymphomaat interim or end- | andAn increase in |
| | | of-treatment assessment | LDi or SDi from |
| | | | nadir |
| | | | 0.5 cm for lesions ≤2 cm |
| | | | 1.0 cm for lesions >2 cm |
| | | | In the setting of splenomegaly, the splenic |
| | | | length must increaseby >50% of the |
| | | | extent of its prior increase beyond |
| | | | baseline (eg, a 15-cm spleen must |
| | | | increase to >16 cm). If no prior |
| | | | splenomegaly, must increase by at least 2 |
| | | | cm from baseline |
| | | | New or recurrent splenomegaly |
| | Nonmeasured | None | New or clear progression of preexisting |
| | lesion | | non-measured lesions |
| | New lesions | New FDG-avid foci consistent | Regrowth of |
| | | with lymphoma rather than | previously resolved |
| | | another etiology (eg, infection, | lesionsA new node |
| | | inflammation). If uncertain | >1.5 cm in any axis |
| | | regarding etiology ofnew | A new extranodal site >1.0 cm in |
| | | lesions, biopsy or interval scan | any axis; if <1.0 cm inany axis, its |
| | | may be considered | presence must be unequivocal and |
| | | | must be attributable to lymphoma |
| | | | Assessable disease of any size |
| | | | unequivocally attributable to |
| | | | lymphoma |
| | Bone marrow | New or recurrent FDG-avid foci | New or recurrent involvement |

5PS = 5-point scale; CT = computed tomography; FDG = fluorodeoxyglucose; IHC = immunohistochemistry; LDi = longest transverse diameter of a lesion; MRI = magnetic resonance imaging; PET = positron emission tomography; PPD = cross product of the LDi and perpendicular diameter; SDi = shortest axisperpendicular to the LDi = SPD, sum of the product of the perpendicular diameters for multiple lesions.
^{a}In Waldeyer's ring or in extranodal sites (eg, GI tract, liver, bone marrow), FDG uptake may be greater than in the mediastinum with complete metabolic response but should be no higher than surrounding normal physiologic uptake (eg, with marrow activation as a result of chemotherapy or myeloid growth factors).
^{b}PET 5PS: 1, no uptake above background; 2, uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma.

Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (eg, liver, spleen, kidneys, lungs), GI involvement, cutaneous lesions, or those noted on palpation. Recently biopsied lesions should not be used as a measured dominant lesion.

Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging.

The Investigator and the BIRC will assess response to study drug at each efficacy timepoint. The following endpoints will be reported:

Progression-free Survival: defined as the time from randomization to the first documentation of progressive disease or death, whichever comes first. The Investigator and the BIRC will determine the progressive disease event in all randomized subjects. PFS as assessed by the BIRC is the primary endpoint for Part 2 of this study. For determination of the sample size, it is assumed that triple combination treatment could improve the median PFS from 12 months (under treatment with tafasitamab+lenalidomide (Duell Haematologica. 2021; 106(9):2417-26) to 23.5 months under the triple combination treatment (plamotamab+tafasitamab+lenalidomide), corresponding to a hazard ratio (HR) of 0.51 with all randomized subjects (it is estimated that 90% of subjects will becentral pathologically confirmed as DLBCL). The log rank test has 90% power with the sample size of 200 (93 events) to preserve the type-I error of 0.025 (one-sided). A loss to follow-up rate of 1.5%/month was assumed for sample size calculations. Subjects will be randomized 1:1, stratified by IPI risk score at baseline (3 to 5 versus 0 to 2), number of lines of prior therapy (1 versus ≥2), and primary refractory (yes versus no). A maximum of 36 primary refractory subjects may be enrolled into the sample size of 200. Enrollment of 200 subjects is estimated to require 27 months with minimum follow-up time of 6 months. The primary efficacy analysis will occur when 93 PFS events per independent review have been observed. An interim analysis for OS at the time of final PFS analysis will be implemented using the group sequential design in addition to the final OS analysis.

Duration of Response: defined as the time from the first response (CR or PR) to progression or death due to any cause among subjects achieving a CR or PR and among CR subjects. DOR will be derived using disease progression as determined by the BIRC in subjects who have a response (CR or PR).

Overall Survival: defined as the time from randomization to death from any cause. All randomized subjects will be followed for up to 5 years for survival. Overall survival will be assessed in the randomized population (Part 2). The one-sided a level for testing OS is at 0.025. It is assumed that the median OS for the Part 2 Arm B is 34 months and that Part 2 Arm A will have 40% improvement from Arm B (ie, median OS=56.6 months, HR=0.60). In addition, assuming completion of enrollment in 27 months, a minimum follow-up time of 24 months, and an OS censoring rate (dropout rate) of approximately 1% per month, the study will result a statistical significance with 60% of power. Once 65% of the information (death events) is available, an interim analysis for OS is planned. Superiority analyses will be performed during the interim and final analyses.

Time to Treatment Failure: defined as the time from randomization to discontinuation of all study treatment for any reason, including disease progression, treatment toxicity, and death. The time to treatment failure will be using disease progression as determined by the Investigator and by the BIRC.

Additionally, adverse events and other safety parameters will be assessed, including incidence of cytokine release syndrome (CRS).

### 5.10 Management of Toxicities

Toxicities of the 3-drug regimen (plamotamab, tafasitamab, and lenalidomide) may be identified during the study. Management of known toxicities such as CRS, infusion-related reactions, hematologic toxicities, and tumor lysis syndrome are outlined as below.

Cytokine Release Syndrome: The ASTCT defines CRS as a supraphysiologic response following any immune therapy that results in the activation or engagement of endogenous or infused T cells and/or other immune effector cells. Symptoms can be progressive, must include fever at the onset, and may include hypotension, capillary leak (hypoxia), and end organ dysfunction (Lee D W, Santomasso B D, Locke F L, et al. ASTCT consensus grading for cytokine release syndrome and neurologic toxicity associated with immune effector cells. Biol Blood Marrow Transplant. 2019; 25(4)625-38).

CRS can present with a variety of symptoms ranging from mild, flu-like symptoms to severe life-threatening manifestations of the inflammatory response. Mild symptoms of CRS include fever, fatigue, headache, rash, arthralgia, and myalgia. More severe cases are characterized by hypotension as well as high fever and can progress to an uncontrolled systemic inflammatory response with vasopressor-requiring circulatory shock, vascular leakage, disseminated intravascular coagulation, and multi-organ system failure (Shimabukuro-Vornhagen A, Gödel P, Subklewe M, et al. Cytokine release syndrome. J Immunother Cancer. 2018; 6(1):56). CRS is more likely to occur after the first dose of plamotamab than subsequent doses, tends to begin somewhat later than hypersensitivity reactions, and is more likely to be associated with hepatic and neurologic complications. Plamotamab associated CRS Symptoms observed in the Phase 1 study include:
Aphasia or word-finding difficulty
Arthralgia
Confusion/mental status changes/delirium
Serum creatinine increase
Diaphoresis
Dizziness
Dyspnea
Fatigue (asthenia, lethargy, malaise)
Fever
Gait disturbance/dysmetria
Headache
Hypotension/hypertension
Hypoxia
Myalgia
Nausea/vomiting
Rigors/chills
Tachycardia
Tachypnea
Seizures
Transaminitis/hyperbilirubinemia
Tremor

CRS toxicity is defined using the ASTCT CRS Consensus Grading (Lee D W, Santomasso B D, Locke F L, et al. ASTCT consensus grading for cytokine release syndrome and neurologic toxicity associated with immune effector cells. Biol Blood Marrow Transplant. 2019; 25(4)625-38). Refer to Table 10 for related definitions.

TABLE 10
American Society for Transplantation and Cellular
Therapy Cytokine Release Syndrome Consensus Grading

| CRS Parameter | Grade 1 | Grade 2 | Grade 3 | Grade 4 |
|---|---|---|---|---|
| Fever^{a} | Temperature ≥38° C. | Temperature ≥38° C. | Temperature ≥38° C. | Temperature ≥38° C. |
| | | With | | |
| Hypotension | None | Not requiring | Requiring a | Requiring |
| | | vasopressors | vasopressor with | multiple |
| | | | or without | vasopressors |
| | | | vasopressin | (excluding |
| | | | | vasopressin) |
| | | And/or^{b} | | |
| Hypoxia | None | Requiring low- | Requiring high- | Requiring |
| | | flow nasal | flow nasal | positive pressure |
| | | cannulac or | cannula^{c}, | (eg, CPAP, |
| | | blow-by | facemask, | BiPAP, |
| | | | nonrebreather | intubation and |
| | | | mask, or Venturi | mechanical |
| | | | mask | ventilation) |

BiPAP = bilevel positive airway pressure; CPAP = continuous positive airway pressure; CRS = cytokine release syndrome; CTCAE = Common Terminology Criteria for Adverse Events. Organ toxicities associated with CRS may be graded according to CTCAE v5.0, but they do not influence CRS grading.
^{a}Fever is defined as temperature ≥38° C. not attributable to any other cause. In subjects who have CRS then receive antipyretic or anticytokine therapy such as tocilizumab or steroids, fever is no longer required to grade subsequent CRS severity. In this case, CRS grading is driven by hypotension and/or hypoxia.
^{b}CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as Grade 3 CRS.
^{c}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

Safety Run-in and Part 2, Arm A (Plamotamab, Tafasitamab and Lenalidomide): Plamotamab can cause CRS. CRS has not been observed in clinical trials of tafasitamab and lenalidomide. However, lenalidomide, through its potential to activate T cells, may potentiate CRS in combination with plamotamab. In addition, to avoid the overlap of CRS caused by plamotamab with tafasitamab-induced, infusion-related reactions, plamotamab should always be given after tafasitamab if administered on the same day. See Section 5.11 for dose modification of lenalidomide and plamotamab in the instance of CRS.

Part 2, Arm B (Tafasitamab and Lenalidomide): If CRS is observed with tafasitamab, treatment guidelines provided for plamotamab should be followed.

Cytokine Release Syndrome Versus Allergic/Hypersensitivity/Infusion-related Reactions: CRS is mechanistically different from allergic/hypersensitive/Infusion-related reactions (Brennan F R, Morton L D, Spindeldreher S, et al. Safety and immunotoxicity assessment of immunomodulatory monoclonal antibodies. MAbs. 2010; 2(3):233-55), although some of the manifestations are common to both AEs and both have been reported to occur with therapeutic antibodies. It is less clear how cytokine release is triggered, although it is likely to be associated with immune cell activation; in this case, CD3-expressing lymphocytes may be the prime effector of CRS.

Use of Tocilizumab for Cytokine Release Syndrome: Tocilizumab (Acterma®) is a therapeutic antibody that interferes with the binding of interleukin-6 (IL-6) to the IL-6 receptor. Tocilizumab can be used to decrease the severity and, possibly, the mortality of severe CRS and early administration may be useful in improving outcomes. Tocilizumab (dose of 8 mg/kg) should be readily available if needed and repeat dosing of tocilizumab may be necessary if signs and symptoms persist or return after initial treatment. In some cases, another treatment (instead of tocilizumab) may be used for CRS.

Fluid Management: CRS can be associated with myocardial dysfunction, pulmonary edema, or capillary leak syndrome (Shimabukuro-Vornhagen A, Godel P, Subklewe M, et al. Cytokine release syndrome. J Immunother Cancer. 2018; 6(1):56). Subjects are monitored for weight gain and intravenous (IV) fluid administration should be monitored in acute cases. Following are guidelines for managing fluids:
If a subject is noted to have had a ≥10% increase in body weight over the previous 2 weeks in association with new or significantly increased bilateral lower extremity edema, dosing should be delayed until this finding is evaluated and, if indicated, treated.
For acute hypotension, IV fluid bolus should be limited to 500 to 1000 mL of normal saline or the equivalent.
If there is not an adequate response to fluids, treatment with tocilizumab (and vasopressors, if necessary) should be considered rather than additional fluid boluses.

Cytokine Release Syndrome Treatment Guidelines by ASTCT CRS Consensus Grade: The CRS treatment guidelines are listed below by grade

### Grade 1

Symptomatic management is recommended. Acetaminophen 650 mg is administered orally as an antipyretic or analgesic and/or diphenhydramine 25 to 50 mg is administered intravenously or orally for rash, pruritus, or other signs and symptoms of hypersensitivity (allergic) reaction if clinically indicated.
Vital signs are measured every 15 minutes or less, or as clinically indicated.
An unscheduled blood sample is obtained for cytokine analysis during the event and approximately 4 hours later, unless scheduled cytokine monitoring is already in progress on the same visit day.
The subject is monitored for worsening of condition; if severity of event increases to a higher grade, infusion is stopped, and steroids are administered.

### Grade 2

Hypotension responsive to fluids not requiring a vasopressor, or mild respiratory symptoms treatable with low-flow oxygen, are signs of Grade 2 toxicity. Older subjects or those with significant comorbidities may be at a higher risk of decompensation in this situation.
Infusion is discontinued and/or administration of additional dexamethasone at a dose of 10 to 20 mg is administered intravenously and/or acetaminophen 650 mg is administered orally and/or diphenhydramine 25 to 50 mg is administered intravenously or orally to treat signs and symptoms.
Once symptoms have resolved, the infusion is restarted at 50% of the baseline rate. If after 1 hour, the subject's symptoms do not return and vital signs are stable, the infusion rate may be increased every 30 minutes as tolerated to the baseline rate.
Vital signs are measured every 15 minutes or less as clinically indicated. The frequency of vital sign assessment may be reduced to every 30 minutes during the infusion, for subjects who can tolerate an increase in the infusion rate back to baseline and maintain normal blood pressure for 30 minutes after the rate increase.
An unscheduled blood sample is obtained for cytokine analysis during the event and approximately 4 hours later, unless scheduled cytokine monitoring is already in progress on the same visit day.
The subject is monitored for worsening of condition; if severity of event increases to a higher grade, infusion is stopped, appropriate treatment is administered, and the guidelines for Grades 3 and 4 reactions are followed if necessary.
Older subjects or those with significant comorbidities may be at higher risk of decompensation in this situation. When reactions occur in these vulnerable subjects, or in the case of rapidly progressing reactions, treatment with tocilizumab 8 mg/kg IV over 1 hour is considered, with or without additional dexamethasone 10 to 20 mg IV (or equivalent).

### Grade 3 and 4

The infusion is stopped and the infusion tubing is disconnected from the subject.
Additional dexamethasone 10 mg is administered intravenously.
Aggressive supportive care is immediately provided. Pressors, fluids, oxygen, epinephrine or bronchodilators, ventilatory support, antipyretics, and analgesics are used as indicated.
Treatment with tocilizumab, 8 mg/kg IV over 1 hour (Hallek M, Cheson B D, Catovsky D, et al. CLL guidelines for diagnosis, indications for treatment, response assessment, and supportive management of CLL. Blood. 2018; 131(25):2745-60), is encouraged for CRS due to the high risk of progression and permanent organ dysfunction.
Hospital admission for observations is almost always indicated, usually to an intensive care unit, since an initial appearance of improvement may quickly yield to rapid decompensation.
Subjects with Grade 3 CRS or infusion reaction may be rechallenged once resolved as described in Section 5.11 and Table 14. Subjects with recurrent Grade 3 or any Grade 4 reaction should not receive further plamotamab treatment but will continue to be followed on the protocol (ie, followed for long-term survival).
An unscheduled blood sample is obtained for cytokine analysis during the event and approximately 4 hours later, unless scheduled cytokine monitoring is already in progress on the same visit day.
An unscheduled sample is obtained for anti-drug antibodies (ADA) testing as close to the onset of the event as possible, at the resolution of the event, and approximately 28 days following the event, unless scheduled cytokine monitoring is already in progress on the same visit day.
Medical personnel and/or medical monitoring personnel should be contacted.

Neurotoxicity: In addition to CRS, another toxicity observed after CAR-T cell therapy and CD3 bispecific antibodies is neurotoxicity. Immune effector cell-associated neurotoxicity syndrome (ICANS) may manifest as delirium, encephalopathy, aphasia, lethargy, difficulty concentrating, agitation, tremor, seizures, and, rarely, cerebral edema (Lee D W, Santomasso B D, Locke F L, et al. ASTCT consensus grading for cytokine release syndrome and neurologic toxicity associated with immune effector cells. Biol Blood Marrow Transplant. 2019; 25(4)625-38). Neurotoxicity is treated separately from CRS, due to its timing and response to treatment.

Neurologic symptoms may occur during or after CRS symptoms, but rarely precede CRS symptoms. The earliest manifestations of ICANS are tremor, dysgraphia, mild difficulty with expressive speech (especially in naming objects), impaired attention, apraxia, and mild lethargy. Headache is a nonspecific symptom, frequently occurring during fever or after chemotherapy in patients without other neurologic dysfunction. Thus, headache alone is not a suitable marker of ICANS. Expressive aphasia, on the other hand, appears to be a very specific symptom of ICANS.

A consensus grading scheme, which is a slightly modified version of the CARTOX-10 screening tool, which incorporates key elements of the mini-mental state examination, the immune effector cell-associated encephalopathy (ICE) score, is used for the grading of ICANS (Table 11). The 10-point ICE screening tool is helpful for assessing subjects for encephalopathy; however, the grading of ICANS requires assessment of the 10-point ICE score as well as evaluation of other neurologic domains, such as level of consciousness, motor symptoms, seizures, and signs of elevated intracranial pressure/cerebral edema, which may occur with or without encephalopathy. The ASTCT ICANS toxicity grading system is shown in Table 12. This grading scale is used to assess neurotoxicity, rather than the CTCAE version 5.0.

TABLE 11
Immune Effector Cell-Associated Encephalopathy Scoring
ICE

Orientation: orientation to year, month, city, hospital: 4 points
Naming: ability to name 3 objects (eg, point to clock, pen, button): 3 points
Following commands: ability to follow simple commands (eg, “Show me 2 fingers” or
“Close your eyes and stick out your tongue”): 1 point
Writing: ability to write a standard sentence (eg, “Our national bird is the bald eagle”): 1
point
Attention: ability to count backwards from 100 by 10: 1 point
ICANS = immune effector cell-associated neurotoxicity syndrome; ICE = Immune Effector Cell-Associated. Encephalopathy (score).
Scoring: 10, no impairment;
7-9, Grade 1 ICANS;
3-6, Grade 2 ICANS;
0-2, Grade 3 ICANS;
0 due to patient unarousable and unable to perform ICE assessment, Grade 4 ICANS

TABLE 12
American Society for Transplantation and Cellular Therapy Immune Effector Cell-
Associated Neurotoxicity Syndrome Consensus Toxicity Grading for Adults

| Neurotoxicity | | | | |
|---|---|---|---|---|
| Domain | Grade 1 | Grade 2 | Grade 3 | Grade 4 |
| ICE score^{a} | 7-9 | 3-6 | 0-2 | 0 (patient is |
| | | | | unarousable and |
| | | | | unable to |
| | | | | perform ICE) |
| Depressed level | Awakens | Awakens to | Awakens only to | Patient is |
| of | spontaneously | voice | tactile stimulus | unarousable or |
| consciousness^{b} | | | | requires |
| | | | | vigorous or |
| | | | | repetitive tactile |
| | | | | stimuli to |
| | | | | arouse. Stupor or |
| | | | | coma |
| Seizure | N/A | N/A | Any clinical | Life-threatening |
| | | | seizure focal or | prolonged |
| | | | generalized that | seizure (>5 |
| | | | resolves rapidly | min); or |
| | | | or | Repetitive |
| | | | nonconvulsive | clinical or |
| | | | seizures on EEG | electrical |
| | | | that resolve with | seizures without |
| | | | intervention | return to |
| | | | | baseline in |
| | | | | between |
| Motor findings^{c} | N/A | N/A | N/A | Deep focal |
| | | | | motor weakness |
| | | | | such as |
| | | | | hemiparesis or |
| | | | | paraparesis |
| Elevated | N/A | N/A | Focal/local | Diffuse cerebral |
| ICP/cerebral | | | edema on | edema on |
| edema | | | neuroimaging^{d} | neuroimaging; |
| | | | | decerebrate or |
| | | | | decorticate |
| | | | | posturing; or |
| | | | | cranial nerve VI |
| | | | | palsy; or |
| | | | | papilledema, or |
| | | | | Cushing's triad |

EEG = electroencephalogram; ICANS = immune effector cell-associated neurotoxicity syndrome; ICE = Immune Effector Cell-Associated Encephalopathy (score); ICP = intracranial pressure; N/A = not applicable; VI = sixth.
ICANS grade is determined by the most severe event (ICE score, level of consciousness, seizure, motor findings, raised ICP/cerebral edema) not attributable to any other cause; for example, a patient with an ICE score of 3 who has a generalized seizure is classified as grade 3 ICANS.
^{a}A patient with an ICE score of 0 may be classified as Grade 3 ICANS if awake with global aphasia, but a patient with an ICE score of 0 may be classified as Grade 4.
^{b}Depressed level of consciousness should be attributable to no other cause (eg, no sedating medication).
^{c}Tremors and myoclonus associated with immune effector cell therapies may be graded according to CTCAE v5.0, but they do not influence ICANS grading.
^{d}Intracranial hemorrhage with or without associated edema is not considered a neurotoxicity feature and is excluded from ICANS grading. It may be graded according to CTCAE v5.0

High levels of IL-6 may directly mediate neurotoxicity. A human anti-IL-6 monoclonal antibody, tocilizumab, may reverse this neurotoxicity, but it would not be expected to cross the blood brain barrier, and efficacy in this setting has not been confirmed. Corticosteroids may be the primary therapy, and tocilizumab may be considered if corticosteroids are ineffective.

### Clinical Management of Neurotoxicity:

Subjects are monitored for signs and symptoms of events (including the use of mental status and neurologic examinations).
If neurotoxicity becomes evident, an unscheduled blood sample is drawn for cytokine analysis at that time, and again 4 hours later, unless scheduled cytokine monitoring is already in progress on the same visit day.
If neurologic toxicity is Grade 4, plamotamab is discontinued.
For Grade 3 neurologic toxicity, drug is withheld until the toxicity has recovered to ≤Grade 1 (mild) and has remained at ≤Grade 1 for at least 3 days before restarting therapy. Therapy is restarted at 75% of the previous dose. The dose is escalate back to full dose at the time of the next dose if ≥Grade 2 toxicity does not recur. If ≥Grade 2 toxicity reoccurs at 75% dose, or if <Grade 2 toxicity takes more than 7 days to resolve, drug is discontinued permanently. If Grade 3 neurologic toxicity persists for more than 7 days, plamotamab is permanently discontinue.
For severe neurologic symptoms, additional dexamethasone is administered at 10 mg intravenously and repeated every 12 hours if symptoms do not abate rapidly.
For recurrent seizures, anticonvulsant therapy may be necessary.

Allergic/Hypersensitivity/Infusion-related Reactions: Infusion-related reactions can occur for tafasitamab as well as plamotamab. An allergic reaction is often caused by a type 1 hypersensitivity mechanism due to immunoglobulin E (IgE)-mediated release of histamines and prostaglandins, although a direct interaction with mast cells and basophils can also occur. However, infusion-related reactions, including serious and fatal reactions, are not uncommon with monoclonal antibody therapeutics and tend to occur most frequently during the first few infusions.

Signs and symptoms usually develop during or shortly after drug infusion in hypersensitivity reactions, are more likely to occur after several doses of the drug and are largely related to histamine release. Typical signs and symptoms include rash/urticaria, flushing, pruritus, fever, dyspnea, cough, and hypotension, although the following may also occur
Arthralgia
Bronchospasm
Confusion/mental status changes/delirium
Dizziness
Fatigue (asthenia, lethargy, malaise)
Hallucinations
Headache
Hypertension
Myalgia
Nausea/vomiting
Rigors/chills
Diaphoresis
Tachycardia

Allergic/hypersensitivity/infusion-related reactions are managed according to institutional practices and guidelines. Allergic/hypersensitivity reactions are defined according to the NCI-CTCAE, Version 5.0 (NCI-CTCAE, 2017) definition of allergic reaction.

Safety Run-in and Part 2, Arm A (Plamotamab, Tafasitamab and Lenalidomide): infusion-related reactions can occur with plamotamab and tafasitamab. To mitigate overlapping toxicity with plamotamab, on a given treatment day, tafasitamab should be given first with a minimum of 2 hours between the end of the tafasitamab infusion and the start of the plamotamab infusion. If a >Grade 1 infusion reaction occurs with tafasitamab, plamotamab is delayed until resolution of all symptoms of the reaction. Depending on the timing of the resolution of all symptoms, it may be necessary to administer the plamotamab the following day.

Part 2, Arm B (Tafasitamab and Lenalidomide): Infusion-related reactions can occur with tafasitamab and lenalidomide. Toxicity management instructions provided in the prescribing information for tafasitamab can be followed. Refer to Section 5.11 for dosage modification.

Hematologic Toxicities: Hematologic toxicities, specifically decreases in absolute neutrophil count (ANC) and platelet count, have been observed in clinical trials of tafasitamab and lenalidomide. Decreases in platelets and ANC can be observed with plamotamab (see Table 13).

TABLE 13
Adverse Events of Neutropenia and Thrombocytopenia with
Tafasitamab/Lenalidomide and Plamotamab (All Grades)

| | Tafasitamab^{a}/Lenalidomide | Plamotamab^{b} |
|---|---|---|
| Neutropenia | 51% | 20.8% |
| Thrombocytopenia | 31% | 24.0% |

^{a}Minjuvi ® [SmPC]. Noord-Holland, Netherlands: Incyte Biosciences Distribution B.V. 2021; Monjuvi ® (tafas) [package insert]. Boston, MA: MORPHOSYS US INC. 2021.
^{b}Plamotamab Investigator's Brochure, 2021

These decreases may worsen in grade or duration when the products are given in combination. Blood counts should be monitored carefully and institutional guidelines followed for the treatment of neutropenia and thrombocytopenia. Treatment may include the use of growth factors and the administration of whole blood/blood product transfusions. Management may also include the delay in the administration of study drugs until the counts return to at least Grade 2 levels. See Section 5.11 for any dose modifications to study drugs.

Safety Run-in and Part 2, Arm A (Plamotamab, Tafasitamab and Lenalidomide): hematologic toxicities, specifically, decreases in ANC and platelet counts can occur in clinical trials of tafasitamab and lenalidomide. Decreases in platelets and ANCs can also occur with plamotamab. These decreases may worsen in grade or duration when the products are given in combination. Blood counts are monitored carefully and institutional guidelines followed for the treatment of neutropenia and thrombocytopenia, which may include growth factors and whole blood/blood product transfusions. Management may also include the delay in the administration of study drugs until the counts return to at least Grade 2 levels. See Section 5.11 for any dose modifications to study drugs.

Part 2, Arm B (Tafasitamab and Lenalidomide): hematologic toxicities can occur with tafasitamab and lenalidomide. Toxicity management instructions provided in the prescribing information for tafasitamab should be followed. See Section 5.11 for any dose modifications to study drugs.

Tumor Lysis Syndrome: tumor lysis syndrome (TLS) is a rare but serious and life-threatening condition arising from rapid release of tumor cellular contents after lysis; resulting metabolic imbalances can lead to acute kidney failure and/or other life-threatening conditions. Subjects with high tumor burden, highly proliferative disease, and/or certain preexisting conditions such as renal disease are at increased risk of TLS.

Safety Run-in and Part 2, Arm A (Plamotamab, Tafasitamab and Lenalidomide): it is unknown whether treatment with plamotamab, tafasitamab, and lenalidomide can cause TLS in R/R DLBCL. The Phase 1 plamotamab monotherapy study in CD20-expressing hematologic malignancies had reported, any-grade TLS in 4 of 96 subjects (4.2%), of which only 2 of the 80 R/R NHL subjects (2.5%) experienced TLS; importantly, no serious or fatal events of TLS was reported with plamotamab monotherapy in R/R DLBCL. Likewise, no events of TLS were reported in the Phase 2 open-label, single-arm, tafasitamab plus lenalidomide study in R/R DLBCL (L-MIND). However, fatal instances of TLS have been reported with lenalidomide in other indications and combinations. All subjects are assessed for risk of TLS according to institutional practices based on laboratory parameters and tumor bulk prior to initiation of the study medications

Prophylaxis, Monitoring, and Treatment of Tumor Lysis Syndrome: prophylaxis should be instituted prior to the start of treatment for subjects at high risk for TLS. Hospitalization may be considered for subjects considered high risk for TLS and/or creatinine clearance of <80 mL/min. Allopurinol (or other xanthine oxidase inhibitors) should be initiated at least 48 hours prior to C1D1 in subjects considered medium to high risk for TLS. Rasburicase is indicated for elevated uric acid levels and monitoring of TLS. During study treatment, all subjects require appropriate laboratory testing prior to administration of study medications including uric acid, potassium, phosphorus, calcium, and creatinine. Laboratory results should be evaluated in real time. If subjects are not able to consume adequate oral fluids, IV fluids should be added.

If a subject experiences laboratory changes or symptoms suggestive of TLS, treatment with plamotamab, tafasitamab and lenalidomide should be interrupted. Study treatment is held until resolution of TLS. If moderate or high risk for TLS remains after resolution, consideration of additional prophylactic measures and/or continued dose interruption or reduction of plamotamab, tafasitamab, or lenalidomide may be considered.

Part 2, Arm B (Tafasitamab and Lenalidomide): the guidelines in disclosed above under “Prophylaxis, Monitoring, and Treatment of Tumor Lysis Syndrome” should be followed for the prophylaxis, monitoring, and treatment of TLS.

### 5.11 Dose Modification Guidelines

Subjects experiencing a significant toxicity are treated with standard medical interventions (e.g., use of filgrastim to treat neutropenia and/or acetaminophen for fever) as appropriate. Standard treatment practices can be used for infusion reactions/Cytokine Release Syndrome (CRS) and neurotoxicity management.

In general, if a >Grade 2 toxicity prevents dosing >14 days and/or if 2 consecutive doses are missed (this applies to weekly and Q2W dosing), the subject may require discontinuation from the plamotamab or tafasitamab treatment. Subjects may reinitiate study drug, even after 2 or more missed consecutive doses of plamotamab or tafasitamab, if it is determined that the benefit outweighs the risk and the toxicity (if applicable) can be controlled by concomitant medication or other means. Medical monitor approval is required for delays of >14 days and/or 2 consecutive doses.

During weekly dosing of plamotamab and/or tafasitamab, if a subject's dose is held for ≥7 days, it should be considered missed. During Q2W dosing of plamotamab and/or tafasitamab, if a subject's dose is held for ≥14 days, it should be considered missed. If a subject's dose is given out of window or delayed for safety concerns, including AEs, it is not considered a protocol deviation.

Plamotamab may be resumed after prolonged dose delay for more than one cycle for reasons other than toxicity (eg, comorbidity, pseudoprogression, or delayed response). Resuming initial treatment course may be allowed on a case-by-case basis if such is considered in the best interest of the subject. As a safety measure, the dose may be lowered to the priming dose for re-titration. Study assessments, including local and/or central labs, are to be collected per the Schedule of Assessments as shown in, for example, Table 14.

For toxicities that could overlap (eg, CRS, infusion reaction, hematologic toxicities), detailed dose modifications by grade are presented in Table 14 below.

TABLE 14
Dose Modifications for Select Toxicities

| Adverse Reaction | Severity | Dosage Modification |
|---|---|---|
| Infusion-related reactions | Grade 2 (moderate) | Interrupt infusion of |
| | | tafasitamab or plamotamab |
| | | immediately and manage |
| | | signs and symptoms. |
| | | Once signs and symptoms |
| | | resolve or reduce to Grade 1, |
| | | resume infusion at no more |
| | | than 50% of the rate at which |
| | | the reaction occurred. If the |
| | | subject does not experience |
| | | further reaction within 1 hour |
| | | and vital signs are stable, the |
| | | infusion rate may be |
| | | increased every 30 minutes as |
| | | tolerated to the rate at which |
| | | the reaction occurred. |
| | | Dosing of other study |
| | | medications is not modified |
| | Grade 3 (severe) | Interrupt infusion of |
| | | tafasitamab or plamotamab |
| | | immediately and manage |
| | | signs and symptoms. |
| | | Once signs and symptoms |
| | | resolve or reduce to Grade 1, |
| | | resume infusion at no more |
| | | than 25% of the rate at which |
| | | the reaction occurred. If the |
| | | subject does not experience |
| | | further reaction within 1 hour |
| | | and vital signs are stable, the |
| | | infusion rate may be |
| | | increased every 30 minutes as |
| | | tolerated to a maximum of |
| | | 50% of the rate at which the |
| | | reaction occurred. |
| | | If the reaction returns after |
| | | the rechallenge, infusion |
| | | should be stopped |
| | | immediately. |
| | | Dosing of other study |
| | | medications is not modified. |
| | Grade 4 (life-threatening) | Stop the infusion |
| | | immediately and permanently |
| | | discontinue: |
| | | Tafasitamab if during or |
| | | immediately following |
| | | tafasitamab infusion. |
| | | Plamotamab if during or |
| | | immediately following |
| | | plamotamab infusion. |
| Cytokine release syndrome | Grade 2 (moderate) | Plamotamab infusion should |
| | | be interrupted immediately |
| | | and signs and symptoms |
| | | managed (see Section 5.10) |
| | | Once signs and symptoms |
| | | resolve or reduce to Grade 1, |
| | | plamotamab infusion is |
| | | resumed at no more than 50% |
| | | of the rate at which the |
| | | reaction occurred. If the |
| | | subject does not experience |
| | | further reaction within 1 hour |
| | | and vital signs are stable, the |
| | | infusion rate may be |
| | | increased every 30 minutes as |
| | | tolerated to the rate at which |
| | | the reaction occurred. |
| | | Tafasitamab and |
| | | lenalidomide dosing is not |
| | | modified. |
| | Grade 3 (severe) & Grade 4 | Plamotamab infusion should |
| | (life-threatening) | be interrupted immediately |
| | | and infusion tubing should be |
| | | disconnected from subject. |
| | | Signs and symptoms should |
| | | be managed. |
| | | The next scheduled dose of |
| | | plamotamab is given at 75% |
| | | of the previous dose, unless |
| | | the reaction occurred on the |
| | | C1D1 dose, in which case |
| | | 100% of the previous dose |
| | | may be given. |
| | | For subsequent plamotamab |
| | | infusions, if: |
| | | Toxicity does not recur or |
| | | recurs at a Grade 1 |
| | | severity, escalate to full dose |
| | | of plamotamab at the time of |
| | | the next dose. |
| | | Toxicity recurs at a Grade 2 |
| | | severity, 75% dosing of |
| | | plamotamab is maintained. |
| | | Toxicity recurs at ≥Grade |
| | | 3 severity at any time or if |
| | | a Grade 2 takes more than 10 |
| | | days to resolve to ≤Grade 1, |
| | | plamotamab is discontinued |
| | | permanently. |
| | | Lenalidomide is also |
| | | modified if a ≥Grade 3 CRS |
| | | event occurs: |
| | | During the first 5 doses of |
| | | plamotamab: Reduce |
| | | current dose of lenalidomide |
| | | by 10 mg (or to 5 mg if |
| | | currently at 10 mg) for |
| | | subsequent doses until C4D1, |
| | | when lenalidomide may be |
| | | increased back to the dose |
| | | prior to CRS. If CRS recurs |
| | | at ≥Grade 2 on subsequent |
| | | reduced dose lenalidomide, |
| | | lenalidomide is permanently |
| | | discontinued. |
| | | After the first 5 doses of |
| | | plamotamab: Reduce |
| | | lenalidomide by 10 mg (or to |
| | | 5 mg if currently at 10 mg) |
| | | for all subsequent doses. If |
| | | CRS recurs at ≥Grade 2 on |
| | | subsequent reduced dose |
| | | lenalidomide, lenalidomide is |
| | | permanently discontinued. |
| | | Tafasitamab dose is not |
| | | modified. |
| Myelosuppression | Platelet count of less | Withhold plamotamab, |
| | than 50,000/μL | tafasitamab, and lenalidomide |
| | Neutrophil count of 1,000/μL | and monitor CBC weekly |
| | or less for at least 7 days OR | until platelet count is |
| | Neutrophil count of 1,000/μL | 50,000/μL or higher. |
| | or less with an increase of | Resume plamotamab and |
| | body temperature to 100.4° F. | tafasitamab at the same dose |
| | (38° C.) or higher OR | and lenalidomide at a reduced |
| | Neutrophil count less than | dose. Refer to lenalidomide |
| | 500/μL | prescribing information for |
| | | dosage modifications. |
| | | If recurrence, repeat drug |
| | | withholding and lenalidomide |
| | | reduction steps until minimal |
| | | lenalidomide dose is reached |
| | | per lenalidomide prescribing |
| | | information. |
| | | If recurrence at minimal |
| | | lenalidomide dose, repeat |
| | | drug withholding step (first |
| | | bullet above) then reduce the |
| | | next scheduled plamotamab |
| | | dose to 75% of the previous |
| | | dose. |
| | | Withhold plamotamab, |
| | | tafasitamab, and lenalidomide |
| | | and monitor CBC weekly |
| | | until neutrophil count is |
| | | 1,000/μL or higher. |
| | | Resume plamotamab and |
| | | tafasitamab at the same dose |
| | | and lenalidomide at a reduced |
| | | dose. Refer to lenalidomide |
| | | prescribing information for |
| | | dosage modifications. |
| | | If recurrence, repeat drug |
| | | withholding (first bullet |
| | | above) and lenalidomide |
| | | reduction steps until minimal |
| | | lenalidomide dose is reached |
| | | per lenalidomide prescribing |
| | | information. |
| | | If recurrence at 5 mg |
| | | lenalidomide, repeat drug |
| | | withholding steps then reduce |
| | | the next scheduled |
| | | plamotamab dose to 75% of |
| | | the previous dose. |
| Febrile Neutropenia | Grade 3 (severe) & Grade 4 | Withhold plamotamab, |
| | (life-threatening) | tafasitamab, and lenalidomide |
| | | and monitor CBC until |
| | | neutrophil count is Grade ≤1. |
| | | Once subject is afebrile, off |
| | | antibiotics and neutrophil |
| | | count is Grade ≤1, resume all |
| | | therapy at the same dose and |
| | | schedule. |
| Neurotoxicity | Grade 3 (severe) | Withhold plamotamab and |
| | | manage signs and symptoms |
| | | until the toxicity has |
| | | recovered to ≤Grade 1 (mild) |
| | | and has remained at ≤Grade |
| | | 1 for at least 3 days before |
| | | restarting therapy (see |
| | | Section 5.10). |
| | | Reduce next scheduled |
| | | plamotamab dose to 75% of |
| | | the previous dose. If ≥Grade |
| | | 2 toxicity does not recur, |
| | | escalate plamotamab to full |
| | | dose in subsequent |
| | | administrations. |
| | | If ≥Grade 2 toxicity recurs |
| | | at 75% dose, or if <Grade 2 |
| | | toxicity takes more than 7 |
| | | days to resolve, permanently |
| | | discontinue plamotamab. |
| | | Tafasitamab and |
| | | lenalidomide dosing is not |
| | | modified. |
| | Grade 4 (life-threatening) | Stop infusion immediately, |
| | | if still infusing and |
| | | permanently discontinue |
| | | plamotamab. |
| AST, ALT, bilirubin, and | Grade 3 (severe) | Withhold plamotamab and |
| alkaline phosphatase in the | | manage signs and symptoms |
| absence of CRS | | until the toxicity has |
| | | recovered to ≤Grade 1 (mild) |
| | | or baseline. |
| | | If a Grade 3 liver toxicity |
| | | persists >14 days and/or 2 |
| | | consecutive doses are missed |
| | | and the toxicity cannot be |
| | | attributed to another cause, |
| | | permanently discontinue |
| | | plamotamab. |
| | | Tafasitamab and |
| | | lenalidomide dosing is not |
| | | modified. |
| | Grade 4 (life-threatening) | Withhold plamotamab, |
| | | tafasitamab, and lenalidomide |
| | | and manage signs and |
| | | symptoms until toxicity |
| | | returns to ≤Grade 1 (mild) or |
| | | baseline |
| | | If a Grade 4 liver toxicity |
| | | persists >3 days, |
| | | permanently discontinue |
| | | plamotamab, tafasitamab, and |
| | | lenalidomide |
| Renal Impairment/Failure | Creatinine clearance 59-30 | Reduce lenalidomide to 10 |
| | mL/min | mg daily on Days 1-21 of |
| | | each cycle. |
| | | Tafasitamab and |
| | | plamotamab dosing is not |
| | | modified. |
| | Creatinine clearance 30 | Reduce lenalidomide to 15 |
| | mL/min not requiring dialysis | mg every other day for Days |
| | | 1-21 of each cycle. |
| | | Tafasitamab and |
| | | plamotamab dosing is not |
| | | modified. |
| | Creatinine Clearance below | Reduce lenalidomide to 5 |
| | 30 mL/min requiring dialysis | mg once daily for Days 1-21 |
| | | of each cycle. On dialysis |
| | | days, administer the dose |
| | | following dialysis. |
| | | Tafasitamab and |
| | | plamotamab dosing is not |
| | | modified. |

ALT = alanine aminotransferase; AST = aspartate aminotransferase; C#D#, eg C1D1 = Cycle # Day #; CBC = complete blood count; CRS = cytokine release syndrome.

For other Grade ≥3 AEs, other dose modifications will be considered on a case-by-case basis.

### 5.12 Results

Subjects treated with a combination of plamotamab, tafasitamab, and lenalidomide are expected to have improved therapeutic outcomes compared to subjects treated with only tafasitamab and lenalidomide.

Disease Assessment at Baseline for DLBCL: prior to the start of the study (17 days prior to the start of the study), a PET-CT scan (5 point scale test) was performed in a subject and resulted in a grade or score of 5 (Table 16). At 61 days after the start of the study, the PET-CT scan showed a score reduction from 5 to 2 for the same subject (Table 17). A PET-CT scan grade of 2 was also observed when measured on day 117 after the start of the study (Table 17). From 177 days after the start of the study up until the end of treatment, the subject had a decrease in PET-CT scan score to 1 (Table 18). Results showed that the subject had complete metabolic response at the end of treatment (and also on or after cycle 2, cycle 4, cycle 6, and/or on or after cycle 8 of the study).

TABLE 16
DLBCL - Disease Assessment at Baseline (Safety Run-in Analysis Set)

| | | Date of | | | | |
|---|---|---|---|---|---|---|
| | | PET-CT or | | | | |
| | | PET Scan | | | | |
| | | (Study | | SPD of 6 | | |
| | | Day)/ | PET 5PS/ | or Fewer | | |
| | | Date of | Bone | Target | | |
| | | CT Scan | marrow | Measurable | Non- | >13 cm |
| | | (Study | uptake on | Lesions | measurable | by CT |
| | Subject | Day) | PET | (mm2) | lesions | Scan? |
| PART | 111-10001 | (−17)/ | 5/ | 4687 | No | No |
| 1A | | (−16) | HIGHER | | | |
| | | | THAN | | | |
| | | | NORMAL | | | |

| | | | | Date of | | |
|---|---|---|---|---|---|---|
| | | Date of | | Bone | | Date of |
| | | Bone | | Marrow | | Bone |
| | | Marrow | # of | Core | # of | Marrow |
| | | Aspirate | Cells | Biopsy | Cells | Core |
| | Size of | (Study | Counted | (Study | Counted | Biopsy |
| | spleen | Day) | in | Day) | in | (Study Day) |
| | (cm) | if Assessed | Aspirate | if Assessed | Aspirate | if Assessed |
| PART | | | | | | N/A |
| 1A | | | | | | |

Study day is based on the first dose date.
SPD = Sum of the Products of Perpendicular Diameters.

TABLE 17
Response Assessment 1

| | | | | | | | SPD of 6 or | | |
|---|---|---|---|---|---|---|---|---|---|
| | | | | | | | Fewer | | |
| | | | Date of PET-CT or | | | New or | Target | All | |
| | | | PET Scan (Study | PET 5PS/Bone | | Recurrent | Measurable | Individual | |
| | | | Day)/Date of CT | marrow uptake | New | Involvement | Lesions | Lesions LDi | Non-measurable |
| Cohort | Subject | Visit | Scan (Study Day) | on PET | Lesion | on Scan | (mm\hat{ }2) | <1.5 cm? | Lesions |
| PART IA | 111-10001 | C2D26 | (61)/(61) | 2/NO CHANGE | No | No | 562 | No | ABSENT/ |
| | | | | FROM STUDY | | | | | NORMAL |
| | | | | BASELINE | | | | | |
| | | C4D26 | (117)/(117) | 2/NO CHANGE | No | No | 553 | No | ABSENT/ |
| | | | | FROM STUDY | | | | | NORMAL |
| | | | | BASELINE | | | | | |
| | | C6D26 | (177)/(177) | 1/NO CHANGE | No | No | 553 | No | ABSENT/ |
| | | | | FROM STUDY | | | | | NORMAL |
| | | | | BASELINE | | | | | |
| | | C8D26 | (233)/(233) | 1/NO CHANGE | No | No | 67 | Yes | ABSENT/ |
| | | | | FROM STUDY | | | | | NORMAL |
| | | | | BASELINE | | | | | |
| | | EOT | (299)/(299) | 1/NO CHANGE | No | No | 18 | Yes | ABSENT/ |
| | | | | FROM STUDY | | | | | NORMAL |
| | | | | BASELINE | | | | | |

5PS = 5-point scale; CT = computed tomography; LDi = longest transverse diameter of a lesion; PET = positron emission tomography; SPD = sum of the product of the perpendicular diameters for multiple lesions.
Study day is based on the first dose date.

TABLE 18
Response Assessment 1

| | | | | | | Bone Marrow Assessment | | | | | | |
|---|---|---|---|---|---|---|---|---|---|---|---|---|
| | | | | | | | | Date of | | | | |
| | | | | | | Date of | | Bone | | | | |
| | | | | | | Bone | # | Marrow | | | Lugano Classification for | |
| | | | Spleen Assessment | | | Marrow | of | Core | | | Response | |
| | | | >13 cm | | | Aspirate | Cells | Biopsy | | | Date of | |
| | | | by | New or | Size of | (Study | Counted | (Study | | | Response | |
| | | | CT | Recurrent | spleen | Day) if | in | Day) if | | Lymphoma | (Study | |
| Cohort | Subject | Visit | Scan? | if Yes? | (cm) | Assessed | Aspirate | Assessed | Cellularity | involvement? | Day) | Response |

| | | | | | | |
|---|---|---|---|---|---|---|
| PART | 111- | C2D26 | No | NOT | (61) | COMPLETE |
| 1A | 10001 | | | DONE | | METABOLIC |
| | | | | | | RESPONSE |
| | | C4D26 | No | NOT | (117) | COMPLETE |
| | | | | DONE | | METABOLIC |
| | | | | | | RESPONSE |
| | | C6D26 | No | NOT | (177) | COMPLETE |
| | | | | DONE | | METABOLIC |
| | | | | | | RESPONSE |
| | | C8D26 | No | NOT | (233) | COMPLETE |
| | | | | DONE | | METABOLIC |
| | | | | | | RESPONSE |
| | | EOT | No | NOT | (299) | COMPLETE |
| | | | | DONE | | METABOLIC |
| | | | | | | RESPONSE |

Study day is based on the first dose date

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

1. A method of treating lymphoma in a subject in need thereof, comprising administering to the subject:
(a) an antibody that binds CD19 (CD19 antibody);
(b) a multispecific antibody, wherein the multispecific antibody comprises a first binding domain that binds to CD3 and a second binding domain that binds to CD20 (CD3×CD20 antibody); and
(c) a compound having the structure: 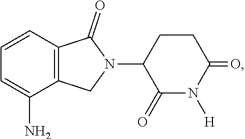 [SMILES: Nc1cccc2c1CN(C1CCC(=O)NC1=O)C2=O]
(Compound A), or a pharmaceutically acceptable salt, solvate, or stereoisomer thereof;
wherein the subject is administered a first dose of the CD19 antibody at least one day prior to administration to the subject of:
(i) a first dose of the Compound A or pharmaceutically acceptable salt, solvate or stereoisomer thereof; or
(ii) a first dose of the CD3×CD20 antibody.

2. The method of claim 1, wherein
(a) the CD19 antibody comprises:
(i) a heavy chain variable (VH) domain comprising a VH complementarity determining region (CDR) 1, a VH CDR2, and a VH CDR3 having an amino acid sequence of SEQ ID NO:1, SEQ ID NO:2, and SEQ ID NO:3, respectively; and
(ii) a light chain variable (VL) domain comprising a VL CDR1, a VL CDR2, and VL CDR3 having an amino acid sequence of SEQ ID NO:4, SEQ ID NO:5, and SEQ ID NO:6, respectively;
(b) the first binding domain of the CD3×CD20 antibody that binds to CD3 comprises:
(i) a VH domain comprising a VH CDR1, a VH CDR2, and a VH CDR3 having an amino acid sequence of SEQ ID NO:7, SEQ ID NO:8, and SEQ ID NO:9, respectively; and
(ii) a VL domain comprising a VL CDR1, a VL CDR2, and VL CDR3 having an amino acid sequence of SEQ ID NO:10, SEQ ID NO:11, and SEQ ID NO:12, respectively; and/or
(c) the second binding domain of the CD3×CD20 antibody that binds to CD20 comprises:
(i) a VH domain comprising a VH CDR1, VH CDR2, and a VH CDR3 having an amino acid sequence of SEQ ID NO:13, SEQ ID NO:14, and SEQ ID NO:15, respectively; and
(ii) a VL domain comprising a VL CDR1, VL CDR2, and VL CDR3 having an amino acid sequence of SEQ ID NO:16, SEQ ID NO:17, and SEQ ID NO:18, respectively.

3. The method of claim 2, wherein:
(a) the CD19 antibody comprises a VH domain having an amino acid sequence that is about 90%, 95%, 98%, 99%, or 100% identical to the amino acid sequence of SEQ ID NO:19;
(b) the CD19 antibody comprises a VL domain having an amino acid sequence that is about 90%, 95%, 98%, 99%, or 100% identical to the amino acid sequence of SEQ ID NO:20;
(c) the first binding domain of the CD3×CD20 antibody that binds to CD3 comprises a VH domain having an amino acid sequence that is about 90%, 95%, 98%, 99%, or 100% identical to the amino acid sequence of SEQ ID NO:21;
(d) the first binding domain of the CD3×CD20 antibody that binds to CD3 comprises a VL domain having an amino acid sequence that is about 90%, 95%, 98%, 99%, or 100% identical to the amino acid sequence of SEQ ID NO:22;
(e) the second binding domain of the CD3×CD20 antibody that binds to CD20 comprises a VH domain having an amino acid sequence that is about 90%, 95%, 98%, 99%, or 100% identical to the amino acid sequence of SEQ ID NO:23; and/or
(f) the second binding domain of the CD3×CD20 antibody that binds to CD20 comprises a VL domain having an amino acid sequence that is about 90%, 95%, 98%, 99%, or 100% identical to the amino acid sequence of SEQ ID NO:24.

4. The method of claim 1, wherein the CD3×CD20 antibody comprises:
(a) a first monomer comprising, from N- to C-terminus, a scFv-linker-CH2-CH3 having the amino acid sequence of SEQ ID NO:25;
(b) a second monomer comprising, from N- to C-terminus, a VH-CH1-hinge-CH2-CH3 having the amino acid sequence of SEQ ID NO:26; and
(c) a third monomer comprising, from N- to C-terminus, a VL-CL having the amino acid sequence of SEQ ID NO:27.

5. The method of claim 1, wherein the compound is

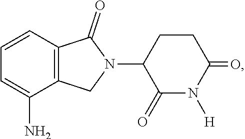 [SMILES: Nc1cccc2c1CN(C1CCC(=O)NC1=O)C2=O]

6. The method of claim 1, wherein the lymphoma is Non-Hodgkin lymphoma.

7.-15. (canceled)

16. The method of claim 1, wherein the method comprises cyclic administration of the CD19 antibody, the Compound A or pharmaceutically acceptable salt, solvate, or stereoisomer thereof, and the CD3×CD20 antibody.

17. The method of claim 16, wherein each cycle of the cyclic administration is 28 days.

18. The method of claim 17, wherein:
the cyclic administration comprises about one cycle, two cycles, three cycles, four cycles, five cycles, six cycles, seven cycles, eight cycles, nine cycles, ten cycles, eleven cycles, twelve cycles, or more than twelve cycles;
the first dose of the CD19 antibody is administered to the subject prior to day 1 of the first cycle of the cyclic administration;
the first dose of the CD19 antibody is administered to the subject at least one day, two days, three days, four days, five days, six days, seven days, eight days, nine days, ten days, or more than ten days prior to day 1 of the first cycle of the cyclic administration;
the first dose of the CD19 antibody is administered to the subject four days prior to day 1 of the first cycle of the cyclic administration;
the first dose of the CD19 antibody is administered to the subject eight days prior to day 1 of the first cycle of the cyclic administration;
the CD19 antibody is administered to the subject four days and eight days prior to day 1 of the first cycle of the cyclic administration;
the CD19 antibody is administered to the subject on day(s) 1, 8, 15, and/or 22 of a cycle of the cyclic administration;
the CD19 antibody is administered to the subject on days 1, 8, 15, and 22 of a cycle of the cyclic administration;
the CD19 antibody is administered to the subject on days 1 and 15 of a cycle of the cyclic administration;
the CD19 antibody is administered to the subject on days 1, 8, 15, and 22 of each of cycles 1-3 of the cyclic administration;
the CD19 antibody is administered to the subject on days 1 and 15 for cycle 4 and onwards of the cyclic administration;
the CD19 antibody is administered to the subject on days 1 and 15 for cycles 4-6 of the cyclic administration;
the CD19 antibody is administered to the subject every 6 to 8 days in a cycle of the cyclic administration;
the Compound A or pharmaceutically acceptable salt, solvate, or stereoisomer thereof is administered to the subject for 21 continuous days of the cyclic administration;
the Compound A or pharmaceutically acceptable salt, solvate, or stereoisomer thereof is administered to the subject from day 1 to day 21 of the cyclic administration;
the Compound A or pharmaceutically acceptable salt, solvate, or stereoisomer thereof is administered to the subject for 21 days followed by seven days of rest in a 28 day cycle of the cyclic administration;
the CD3×CD20 antibody is administered to the subject on day(s) 1, 8, 15, and/or 22 of a cycle of the cyclic administration;
the CD3×CD20 antibody is administered to the subject on days 1, 8, 15, and 22 of a cycle of the cyclic administration;
the CD3×CD20 antibody is administered to the subject on days 1 and 15 of a cycle of the cyclic administration;
the CD3×CD20 antibody is administered to the subject on days 1, 8, 15, and 22 of cycle 1 and cycle 2 of the cyclic administration;
the CD3×CD20 antibody is administered to the subject on days 1 and 15 for cycle 3 and onwards of the cyclic administration;
the CD3×CD20 antibody is administered to the subject on days 1 and 15 for cycles 3-6 of the cyclic administration; or
the CD3×CD20 antibody is administered to the subject every 6 to 8 days in a cycle of the cyclic administration.

19.-40. (canceled)

41. The method of claim 1, wherein the CD19 antibody is administered to the subject in an amount of about 1 mg/kg to about 20 mg/kg per day.

42. The method of claim 41, wherein:
the CD19 antibody is administered to the subject in an amount of about 12 mg/kg per day;
the CD19 antibody is administered to the subject in an amount of about 5 mg/kg per day;
or the CD19 antibody is administered to the subject in an amount of about 10 mg/kg per day.

43.-45. (canceled)

46. The method of claim 1, wherein:
the Compound A or pharmaceutically acceptable salt, solvate, or stereoisomer thereof is administered to the subject in an amount of about 2.5 mg, 5 mg, 10 mg, 15 mg, 20 mg, or 25 mg per day;
the Compound A or pharmaceutically acceptable salt, solvate, or stereoisomer thereof is administered to the subject in an amount of about 2.5 mg per day;
the Compound A or pharmaceutically acceptable salt, solvate, or stereoisomer thereof is administered to the subject in an amount of about 5 mg per day;
the Compound A or pharmaceutically acceptable salt, solvate, or stereoisomer thereof is administered to the subject in an amount of about 10 mg per day;
the Compound A or pharmaceutically acceptable salt, solvate, or stereoisomer thereof is administered to the subject in an amount of about 15 mg per day;
the Compound A or pharmaceutically acceptable salt, solvate, or stereoisomer thereof is administered to the subject in an amount of about 20 mg per day;
the Compound A or pharmaceutically acceptable salt, solvate, or stereoisomer thereof is administered to the subject in an amount of about 25 mg per day;
the CD3×CD20 antibody is administered to the subject in an amount of about 0.8 mg to about 100 mg per day;
the CD3×CD20 antibody is administered to the subject in an amount of about 0.8 mg to about 50 mg per day;
the CD3×CD20 antibody is administered to the subject in an amount of about 0.8 mg to about 20 mg per day;
the CD3×CD20 antibody is administered to the subject in an amount of about 0.8 mg per day;
the CD3×CD20 antibody is administered to the subject in an amount of about 2 mg per day;
the CD3×CD20 antibody is administered to the subject in an amount of about 20 mg per day;
the CD3×CD20 antibody is administered to the subject in an amount of about 35 mg per day;
the CD3×CD20 antibody is administered to the subject in an amount of about 50 mg per day.

47.-60. (canceled)

61. The method of claim 16, wherein the first dose of the CD3×CD20 antibody is on day 1 of the first cycle of the cyclic administration.

62. The method of claim 1, wherein:
the CD3×CD20 antibody is administered to the subject in an amount of about 0.8 mg on day 1 of the first cycle, about 2 mg on day 8 of the first cycle, about 20 mg on days 15 and 22 of the first cycle, and about 20 mg per day for any subsequent cycles;
the CD3×CD20 antibody is administered to the subject in an amount of about 0.8 mg on day 1 of the first cycle, about 2 mg on day 8 of the first cycle, about 20 mg on day 15 of the first cycle, about 35 mg on day 22 of the first cycle, and about 50 mg per day for any subsequent cycles;
the first dose of the Compound A or pharmaceutically acceptable salt, solvate, or stereoisomer thereof is on day 1 of the first cycle of the cyclic administration;
the first dose of the CD3×CD20 antibody and the first dose of the Compound A or pharmaceutically acceptable salt, solvate, or stereoisomer thereof are both on day 1 of the first cycle of the cyclic administration;
the CD19 antibody is administered to the subject once a week in a cycle of the cyclic administration;
the CD19 antibody is administered to the subject once a week for cycles 1-3 of the cyclic administration;
the CD19 antibody is administered to the subject every two weeks in a cycle of the cyclic administration;
the CD19 antibody is administered to the subject every two weeks for cycles 4 and onwards of the cyclic administration;
the CD3×CD20 antibody is administered to the subject once a week in a cycle of the cyclic administration;
the CD3×CD20 antibody is administered to the subject once a week for cycles 1 and 2 of the cyclic administration;
the CD3×CD20 antibody is administered to the subject every two weeks in a cycle of the cyclic administration;
the CD3×CD20 antibody is administered to the subject every two weeks for cycles 3 and onwards of the cyclic administration;
the CD3×CD20 antibody and the CD19 antibody are each administered to the subject in at most 4 days in a cycle of the cyclic administration.

63.-74. (canceled)

75. The method of claim 1, wherein;
the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 20 mg, wherein about 20 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment, and wherein each cycle of the cyclic administration is 28 days;
the method comprises cyclic administration of the CD3×CD20 antibody to the subject, wherein the first cycle comprises administration of the CD3×CD20 antibody to the subject on day 1 of about 0.8 mg, on about day 8 of about 2 mg, on about day 15 of about 20 mg, and on about day 22 of about 35 mg, wherein about 50 mg of the CD3×CD20 antibody is administered to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment, and wherein each cycle of the cyclic administration is 28 days;
the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 20 mg, wherein about 20 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment, and wherein each cycle of the cyclic administration is 28 days;
the method comprises cyclic administration of the CD3×CD20 antibody to the subject, wherein the first cycle comprises administration of the CD3×CD20 antibody to the subject on day 1 of about 0.8 mg, on day 8 of about 2 mg, on day 15 of about 20 mg, and on day 22 of about 35 mg, wherein about 50 mg of the CD3×CD20 antibody is administered to the subject on day 1 and every 7 days in the first 2 cycles of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment, and wherein each cycle of the cyclic administration is 28 days;
the method comprises cyclic administration of the CD19 antibody to the subject, wherein from about 10 mg/kg to about 15 mg/kg of the CD19 antibody is administered to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during a course of treatment, wherein from about 10 mg/kg to about 15 mg/kg of the CD19 antibody is administered to the subject 8 days and 4 days prior to day 1 of a first cycle of the cyclic administration, and wherein each cycle of the cyclic administration is 28 days;
the method comprises cyclic administration of the CD19 antibody to the subject, wherein about 12 mg/kg of the CD19 antibody is administered to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during a course of treatment, wherein about 12 mg/kg of the CD19 antibody is administered to the subject 8 days and 4 days prior to day 1 of a first cycle of the cyclic administration, and wherein each cycle of the cyclic administration is 28 days; or
the method comprises cyclic administration of the Compound A or pharmaceutically acceptable salt, solvate, or stereoisomer thereof to the subject, wherein about 25 mg of the Compound A or pharmaceutically acceptable salt, solvate, or stereoisomer thereof is administered to the subject on day 1 and on every day for 21 days followed by 7 days of rest in each cycle of the cyclic administration, and wherein each cycle of the cyclic administration is 28 days.

76.-81. (canceled)

82. The method of claim 1, wherein the method comprises cyclic administration of the CD19 antibody, the CD3×CD20 antibody, and the Compound A or pharmaceutically acceptable salt, solvate, or stereoisomer thereof to the subject, and wherein the method comprises:
administering from about 0.6 to about 1 mg of the CD3×CD20 antibody to the subject on day 1, from about 1.8 mg to about 2.2 mg on about day 8, and from about 18 mg to about 22 mg on about day 15 and 22 of the first cycle; administering about 20 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 12 to 16 days in cycle 3 and in any subsequent cycle during a course of treatment;
administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and
administering about 25 mg of the Compound A or pharmaceutically acceptable salt, solvate, or stereoisomer thereof to the subject on day 1 and on every day for 21 days followed by 7 days of rest in each cycle of the cyclic administration; and
wherein each cycle of the cyclic administration is 28 days;
administering from about 0.6 to about 1 mg of the CD3×CD20 antibody to the subject on day 1, from about 1.8 mg to about 2.2 mg on about day 8, from about 18 mg to about 22 mg on about day 15, and from about 33 mg to about 36 mg on about day 22 of the first cycle; administering about 50 mg of the CD3×CD20 antibody to the subject on day 1 and every 6 to 8 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 12 to 16 days in cycle 3 and in any subsequent cycle during a course of treatment; administering from about 10 mg/kg to about 15 mg/kg of the CD19 antibody to the subject on day 1 and every 6 to 8 days for the first 3 cycles of the cyclic administration, wherein four doses of the CD19 antibody is administered to the subject in each of the first 3 cycles, wherein from about 10 mg/kg to about 15 mg/kg of the CD19 antibody is administered to the subject on day 1 and every 12 to 16 days in cycle 4 and in any subsequent cycle during the course of treatment, wherein from about 10 mg/kg to about 15 mg/kg of the CD19 antibody is administered to the subject 8 days and 4 days prior to day 1 of a first cycle of the cyclic administration; and administering about 25 mg of the Compound A or pharmaceutically acceptable salt, solvate, or stereoisomer thereof to the subject on day 1 and on every day for 21 days followed by 7 days of rest in each cycle of the cyclic administration; and wherein each cycle of the cyclic administration is 28 days;
administering about 0.8 mg of the CD3×CD20 antibody to the subject on day 1, about 2 mg on about day 8, and about 20 mg on day 15 and 22 of the first cycle; administering about 20 mg of the CD3×CD20 antibody to the subject on day 1 and every 7 days in the second cycle of the cyclic administration, wherein four doses of the CD3×CD20 antibody is administered to the subject in each of the first 2 cycles, wherein about 20 mg of the CD3×CD20 antibody is administered to the subject on day 1 and every 14 days in cycle 3 and in any subsequent cycle during a course of treatment; administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days;
administering about 0.8 mg of the CD3×CD20 antibody to the subject on day 1, about 2 mg on about day 8, about 20 mg on day 15, and about 35 mg on day 22 of the first cycle; administering about 50 mg of the CD3×CD20 antibody to the subject on day 1 and every 7 days in the second cycle of the cyclic administration, wherein four doses of the CD3×CD20 antibody is administered to the subject in each of the first 2 cycles, wherein about 50 mg of the CD3×CD20 antibody is administered to the subject on day 1 and every 14 days in cycle 3 and in any subsequent cycle during a course of treatment; administering about 12 mg/kg of the CD19 antibody to the subject on day 1 and every 7 days for the first 3 cycles of the cyclic administration, wherein four doses of the CD19 antibody is administered to the subject in each of the first 3 cycles, wherein about 12 mg/kg of the CD19 antibody is administered to the subject on day 1 and every 14 days in cycle 4 and in any subsequent cycle during the course of treatment, wherein about 12 mg/kg of the CD19 antibody is administered to the subject 8 days and 4 days prior to day 1 of a first cycle of the cyclic administration; and administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days;
administering about 0.8 mg of the CD3×CD20 antibody to the subject on day 1, about 2 mg on day 8, and about 20 mg on days 15 and 22 of the first cycle; administering about 20 mg of the CD3×CD20 antibody to the subject on days 1, 8, 15, and 22 of the second cycle of the cyclic administration, wherein about 20 mg of the CD3×CD20 antibody is administered to the subject on days 1 and 15 of cycle 3 and of any subsequent cycle during a course of treatment; administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days; or
administering about 0.8 mg of the CD3×CD20 antibody to the subject on day 1, about 2 mg on day 8, about 20 mg on day 15, and about 35 mg on day 22 of the first cycle; administering about 50 mg of the CD3×CD20 antibody to the subject on days 1, 8, 15, and 22 of the second cycle of the cyclic administration, wherein about 50 mg of the CD3×CD20 antibody is administered to the subject on days 1 and 15 of cycle 3 and of any subsequent cycle during a course of treatment; administering about 12 mg/kg of the CD19 antibody to the subject on days 1, 8, 15, and 22 of the first 3 cycles of the cyclic administration, wherein about 12 mg/kg of the CD19 antibody is administered to the subject on days 1 and 15 of cycle 4 and of any subsequent cycle during the course of treatment, wherein about 12 mg/kg of the CD19 antibody is administered to the subject 8 days and 4 days prior to day 1 of a first cycle of the cyclic administration; and administering about 25 mg of the Compound A or pharmaceutically acceptable salt, solvate, or stereoisomer thereof to the subject on day 1 and on every day for 21 days followed by 7 days of rest in each cycle of the cyclic administration during the course of treatment; and wherein each cycle of the cyclic administration is 28 days.

83.-87. (canceled)

88. The method of claim 1, wherein the CD19 antibody is tafasitamab.

89. The method of claim 1, wherein the CD3×CD20 antibody is plamotamab.

90. The method of claim 1, wherein:
the Compound A or pharmaceutically acceptable salt, solvate, or stereoisomer thereof is administered orally to the subject;
the Compound A or pharmaceutically acceptable salt, solvate, or stereoisomer thereof is administered in a capsule or tablet to the subject; and/or
the CD19 antibody is administered intravenously.

91.-92. (canceled)

93. The method of claim 16, wherein:
the CD19 antibody is not administered to the subject on day 4 of the first cycle of the cyclic administration; and/or
the CD3×CD20 antibody is not administered to the subject on day 4 of the first cycle of the cyclic administration.

94. (canceled)

95. The method of claim 16, wherein the method further comprises determining positron emission tomography-computed tomography (PET-CT) after every two cycles of the cyclic administration.

96. The method of claim 1, wherein the subject has received a prior CAR-T therapy.

97. The method of claim 1, wherein the method results in enhanced therapeutic efficacy relative to administration of both the CD19 antibody and the Compound A or pharmaceutically acceptable salt, solvate, or stereoisomer thereof to the subject, but not the CD3×CD20 antibody.

98. The method of claim 97, wherein the enhanced therapeutic efficacy is measured by increased overall survival time, increased progression-free survival, or a decrease in the number of cancer cells in a biological sample obtained from the subject as compared to a reference.

99.-108. (canceled)

109. The method of claim 1, wherein the subject received a prior treatment for lymphoma.

110. The method of claim 109, wherein the prior treatment comprises chemoimmunotherapy, an anti-CD20 antibody, or a combination thereof.

111. A method of treating lymphoma in a subject in need thereof, comprising administering to the subject:
(a) an antibody that binds CD19 (CD19 antibody);
(b) a multispecific antibody, wherein the multispecific antibody comprises a first binding domain that binds to CD3 and a second binding domain that binds to CD20 (CD3×CD20 antibody); and
(c) a compound having the structure: 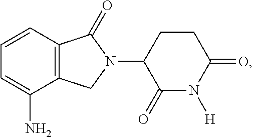 [SMILES: Nc1cccc2c1CN(C1CCC(=O)NC1=O)C2=O]
(Compound A), or a pharmaceutically acceptable salt, solvate, or stereoisomer thereof.

112. The method of claim 111, wherein
(a) the CD19 antibody comprises:
(i) a heavy chain variable (VH) domain comprising a VH complementarity determining region (CDR) 1, a VH CDR2, and a VH CDR3 having an amino acid sequence of SEQ ID NO:1, SEQ ID NO:2, and SEQ ID NO:3, respectively; and
(ii) a light chain variable (VL) domain comprising a VL CDR1, a VL CDR2, and VL CDR3 having an amino acid sequence of SEQ ID NO:4, SEQ ID NO:5, and SEQ ID NO:6, respectively;
(b) the multispecific antibody comprises:
(i) an anti-CD3e heavy chain having an amino acid sequence of SEQ ID NO:30, an anti-CD3e light chain having an amino acid sequence of SEQ ID NO:31, an anti-CD20 heavy chain having an amino acid sequence of SEQ ID NO:32 and an anti-CD20 light chain having an amino acid sequence of SEQ ID NO:33, or
(ii) an anti-CD3e heavy chain having an amino acid sequence of SEQ ID NO:34, an anti-CD3e light chain having an amino acid sequence of SEQ ID NO:35, an anti-CD20 heavy chain having an amino acid sequence of SEQ ID NO:36 and an anti-CD20 light chain having an amino acid sequence of SEQ ID NO:37, or
(iii) an anti-CD3 heavy chain having an amino acid sequence of SEQ ID NO:38, a light chain having an amino acid sequence of SEQ ID NO:39, and an anti-CD20 heavy chain having an amino acid sequence of SEQ ID NO:40, or
(iv) an anti-CD20/CD3 heavy chain having an amino acid sequence of SEQ ID NO:41, an anti-CD3e light chain having an amino acid sequence of SEQ ID NO:42, an anti-CD20 heavy chain having an amino acid sequence of SEQ ID NO:43 and an anti-CD20 light chain having an amino acid sequence of SEQ ID NO:44.

113. The method of claim 112, wherein
(a) the CD19 antibody comprises a VH domain having an amino acid sequence that is about or at least about 90%, 95%, 98%, 99%, or 100% identical to the amino acid sequence of SEQ ID NO:19;
(b) the CD19 antibody comprises a VL domain having an amino acid sequence that is about or at least about 90%, 95%, 98%, 99%, or 100% identical to the amino acid sequence of SEQ ID NO:20.

114. (canceled)

115. The method of claim 111, wherein the lymphoma is Non-Hodgkin lymphoma.

116.-119. (canceled)

120. The method of claim 1, wherein:
the subject achieves a complete metabolic response as determined by a positron emission tomography (PET)-computed tomography (CT) scan;
the subject achieves a complete metabolic response on or after: cycle 2, cycle 4, cycle 6, cycle 8, and/or end of treatment;
the subject achieves a complete metabolic response on or after: day 26 of cycle 2, day 26 of cycle 4, day 26 of cycle 6, day 26 of cycle 8, and/or end of treatment; or
the subject achieves a complete metabolic response on or after: 61 days, 117 days, 177 days, 233 days, or 299 days from a first administration of the multispecific antibody to the subject.

121.-123. (canceled)


SEQUENCE LISTING (44 sequences)

SEQ ID NO: 1 - protein, 5 aa, Synthetic construct
  source 1..5: note = Anti-CD19 VH CDR1
SYVMH

SEQ ID NO: 2 - protein, 17 aa, Synthetic construct
  source 1..17: note = Anti-CD19 VH CDR2
YINPYNDGTKYNEKFQG

SEQ ID NO: 3 - protein, 12 aa, Synthetic construct
  source 1..12: note = Anti-CD19 VH CDR3
GTYYYGTRVFDY

SEQ ID NO: 4 - protein, 16 aa, Synthetic construct
  source 1..16: note = Anti-CD19 VL CDR1
RSSKSLQNVNGNTYLY

SEQ ID NO: 5 - protein, 7 aa, Synthetic construct
  source 1..7: note = Anti-CD19 VL CDR2
RMSNLNS

SEQ ID NO: 6 - protein, 9 aa, Synthetic construct
  source 1..9: note = Anti-CD19 VL CDR3
MQHLEYPIT

SEQ ID NO: 7 - protein, 5 aa, Synthetic construct
  source 1..5: note = Anti-CD3 VH CDR1
TYAMN

SEQ ID NO: 8 - protein, 19 aa, Synthetic construct
  source 1..19: note = Anti-CD3 VH CDR2
RIRSKYNNYATYYADSVKG

SEQ ID NO: 9 - protein, 14 aa, Synthetic construct
  source 1..14: note = Anti-CD3 VH CDR3
HGNFGDSYVSWFAY

SEQ ID NO: 10 - protein, 14 aa, Synthetic construct
  source 1..14: note = Anti-CD3 VL CDR1
GSSTGAVTTSNYAN

SEQ ID NO: 11 - protein, 7 aa, Synthetic construct
  source 1..7: note = Anti-CD3 VL CDR2
GTNKRAP

SEQ ID NO: 12 - protein, 9 aa, Synthetic construct
  source 1..9: note = Anti-CD3 VL CDR3
ALWYSNHWV

SEQ ID NO: 13 - protein, 5 aa, Synthetic construct
  source 1..5: note = Anti-CD20 VH CDR1
SYNMH

SEQ ID NO: 14 - protein, 17 aa, Synthetic construct
  source 1..17: note = Anti-CD20 VH CDR2
AIYPGNGDTSYNQKFQG

SEQ ID NO: 15 - protein, 12 aa, Synthetic construct
  source 1..12: note = Anti-CD20 VH CDR3
STYYGGDWYFNV

SEQ ID NO: 16 - protein, 10 aa, Synthetic construct
  source 1..10: note = Anti-CD20 VL CDR1
RASSSVSYIH

SEQ ID NO: 17 - protein, 7 aa, Synthetic construct
  source 1..7: note = Anti-CD20 VL CDR2
ATSNLAS

SEQ ID NO: 18 - protein, 9 aa, Synthetic construct
  source 1..9: note = Anti-CD20 VL CDR3
QQWTSNPPT

SEQ ID NO: 19 - protein, 121 aa, Synthetic construct
  source 1..121: note = Anti-CD19 VH
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 20 - protein, 112 aa, Synthetic construct
  source 1..112: note = Anti-CD19 VL
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 21 - protein, 125 aa, Synthetic construct
  source 1..125: note = Anti-CD3 VH
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 22 - protein, 109 aa, Synthetic construct
  source 1..109: note = Anti-CD3 VL
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 23 - protein, 121 aa, Synthetic construct
  source 1..121: note = Anti-CD20 VH
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSS

SEQ ID NO: 24 - protein, 106 aa, Synthetic construct
  source 1..106: note = Anti-CD20 VL
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 25 - protein, 485 aa, Synthetic construct
  source 1..485: note = scFv-linker-CH2-CH3
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 450 aa, Synthetic construct
  source 1..450: note = VH-CH1-hinge-CH2-CH3
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 27 - protein, 213 aa, Synthetic construct
  source 1..213: note = VL-CL
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 451 aa, Synthetic construct
  source 1..451: note = Anti-CD19 heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 219 aa, Synthetic construct
  source 1..219: note = Anti-CD19 light chain
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 449 aa, Synthetic construct
  source 1..449: note = mosunetuzumab anti-CD3e heavy chain
EVQLVQSGAEVKKPGASVKVSCKASGYTFTNYYIHWVRQAPGQGLEWIGWIYPGDGNTKYNEKEKGRATLTADTSTSTAYLELSSLRSEDTAVYYCARDSYSNYYFDYWGQGTLVTVSSASTKGPSVEPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVELFPPKPKDTLMISRTPEVTCVVVDVSHEDPEVKENWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVESCSVMHEALHNHYTQKSLSLSPGK

SEQ ID NO: 31 - protein, 219 aa, Synthetic construct
  source 1..219: note = mosunetuzumab anti-CD3e light chain
DIVMTQSPDSLAVSLGERATINCKSSQSLLNSRTRKNYLAWYQQKPGQPPKLLIYWASTRESGVPDRESGSGSGTDETLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSENRGEC

SEQ ID NO: 32 - protein, 452 aa, Synthetic construct
  source 1..452: note = mosunetuzumab anti-CD20 heavy chain
EVQLVESGGGLVQPGGSLRLSCAASGYTFTSYNMHWVRQAPGKGLEWVGAIYPGNGDTSYNQKEKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVESCSVMHEALHNHYTQKSLSLSPGK

SEQ ID NO: 33 - protein, 213 aa, Synthetic construct
  source 1..213: note = mosunetuzumab anti-CD20 light chain
DIQMTQSPSSLSASVGDRVTITCRASSSVSYMHWYQQKPGKAPKPLIYAPSNLASGVPSRFSGSGSGTDFTLTISSLQPEDFATYYCQQWSENPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 454 aa, Synthetic construct
  source 1..454: note = epcoritamab anti-CD3e heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 35 - protein, 215 aa, Synthetic construct
  source 1..215: note = epcoritamab anti-CD3e light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 451 aa, Synthetic construct
  source 1..451: note = epcoritamab anti-CD20 heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 37 - protein, 214 aa, Synthetic construct
  source 1..214: note = epcoritamab anti-CD20 light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 449 aa, Synthetic construct
  source 1..449: note = odronextamab anti-CD3 heavy chain
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 39 - protein, 214 aa, Synthetic construct
  source 1..214: note = odronextamab light chain
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 453 aa, Synthetic construct
  source 1..453: note = odronextamab anti-CD20 heavy chain
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 674 aa, Synthetic construct
  source 1..674: note = glofitamab anti-CD20/CD3 heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 232 aa, Synthetic construct
  source 1..232: note = glofitamab anti-CD3e light chain
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 448 aa, Synthetic construct
  source 1..448: note = glofitamab anti-CD20 heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLLSPGK

SEQ ID NO: 44 - protein, 219 aa, Synthetic construct
  source 1..219: note = glofitamab anti-CD20 light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC